(19) United States
(12) Patent Application Publication
(10) Pub. No.: US 2025/0135030 A1
(43) Pub. Date: May 1, 2025

(54) DEPLETION OF CELLS BY CRISPR NUCLEASES

(71) Applicant: BRAIN BIOTECH AG, Zwingenberg (DE)

(72) Inventors: Paul SCHOLZ, Zwingenberg (DE); Christian ZUREK, Zwingenberg (DE)

(73) Assignee: BRAIN BIOTECH AG, Zwingenberg (DE)

(21) Appl. No.: 18/730,505

(22) PCT Filed: Jan. 18, 2024
(86) PCT No.: PCT/EP2023/051082

§ 371 Date: Jul. 19, 2024

(30) Foreign Application Priority Data
Jan. 19, 2022 (EP) 22152248.5

(51) Int. Cl. A61K 48/00; C12N 9/22; C12N 15/11; C12N 15/85; C12N 15/90

(52) U.S. Cl. CPC A61K 48/005; C12N 9/22; C12N 15/111; C12N 15/85; C12N 15/90; C12N 2310/20

(57) ABSTRACT

The present invention relates to a method for the genome editing of cells at a target locus by homology directed repair (HDR) within a cell population and concurrently enriching within said cell population the HDR-genome edited cells. The present invention also relates to a method for the selective depletion of cells comprising a target locus within a cell population.

The present invention relates to a method for the genome editing of cells at a target locus by homology directed repair (HDR) within a cell population and concurrently enriching within said cell population the HDR-genome edited cells, wherein the method comprises (A) introducing into the cells within the cell population one or more nucleic acid molecules, said one or more nucleic acid molecules encoding in expressible form (i) a CRISPR nuclease comprising of consisting of (a) an amino acid sequence of any one of SEQ ID NO: 1, 2 or 3; (b) an amino acid sequence being encoded by the nucleotide sequence of SEQ ID NO: 4, 5, 6 or 7; (c) an amino acid sequence being at least 80% identical to the amino acid sequence of (a), or (d) an amino acid sequence being encoded by a nucleic acid sequence being at least 80% identical to the nucleotide sequence of (b), (ii) a guide RNA comprising (a) a first segment comprising a nucleotide sequence that is complementary to a sequence at the target locus; and (a) a second segment that interacts with the CRISPR nuclease of (i), and a nucleic acid molecule comprising, consisting of, or encoding (iii) a donor template with homology to the target locus; or (A′) introducing into the cells within the cell population (i) a CRISPR nuclease comprising or consisting of (a) an amino acid sequence of any one of SEQ ID NO: 1, 2 or 3; (b) an amino acid sequence being encoded by the nucleotide sequence of SEQ ID NO: 4, 5, 6 or 7; (c) an amino acid sequence being at least 80% identical to the amino acid sequence of (a), or (d) an amino acid sequence being encoded by a nucleic acid sequence being at least 80% identical to the nucleotide sequence of (b), a nucleic acid molecule, said nucleic acid molecule encoding in expressible form (ii) a guide RNA comprising (a) a first segment comprising a nucleotide sequence that is complementary to a sequence at the target locus; and (a) a second segment that interacts with the CRISPR nuclease of (i), and a nucleic acid molecule comprising, consisting of, or encoding (iii) a donor template with homology to the target locus; or (A″) introducing into the cells within the cell population (i) a ribonucleoprotein complex (RNP) comprising or consisting of a CRISPR nuclease comprising or consisting of (a) an amino acid sequence of any one of SEQ ID NO: 1, 2 or 3; (b) an amino acid sequence being encoded by the nucleotide sequence of SEQ ID NO: 4, 5, 6 or 7; (c) an amino acid sequence being at least 80% identical to the amino acid sequence of (a), or (d) an amino acid sequence being encoded by a nucleic acid sequence being at least 80% identical to the nucleotide sequence of (b), in complex with a guide RNA comprising (a) a first segment comprising a nucleotide sequence that is complementary to a sequence at the target locus; and (a) a second segment that interacts with the CRISPR nuclease (ii) a nucleic acid molecule comprising, consisting of, or encoding a donor template with homology to the target locus; and (B) culturing the cells under conditions wherein the target locus with the cells of the cell population becomes genome edited by homology directed repair (HDR) and the CRISPR nuclease concurrently enriches within said cell population the HDR-genome edited cells.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The genome editing technology CRISPR enables genome editing in a broad range of cells and organisms. The CRISPR-based technique substantially streamlined the targeted gene modification in different prokaryotic and eukaryotic cells. However, depending on the cell type and the targeted DNA region within the genome, CRISPR can be inefficient, resulting in less than one percent of edited mammalian cells.

Successful genome editing with CRISPR (or other programmable nucleases) requires three sequential preconditions: (1) Efficient delivery of the CRISPR-encoding genes into the target cell (transfection/transduction efficiency); (2) efficient expression of the CRISPR-components (CRISPR nuclease and the CRISPR-RNAs); and (3) targeting of the gene of interest (GOI) by CRISPR ribonucleoprotein complexes and repair of the DNA by cell's own repair pathways.

The overall success of genome editing depends on the efficiency of each of these single steps. The frequency of successful editing events within a transfected cell population thus correlates with an efficient gene delivery (step 1), high nuclease expression and the formation of functional ribonucleoprotein complexes (step 2) and finally the introduction of double-strand DNA breaks (DSB) followed by the repair of the DSBs in the cell (step 3).

Several approaches have been developed to isolate the subpopulations of cells that express the nuclease, e.g. CRISPR vectors that enable Fluorescence or Magnetic-Activated Cell Sorting (FACS and MACS). These and other available methods allow the enrichment of either transfected cells or cells that express the CRISPR nuclease, which is necessary but not sufficient to obtain edited cells. This is due to the fact that the expression of the nuclease in a given cell does not necessarily imply the formation of functional CRISPR ribonucleoprotein complexes and the introduction of mutations at the targeted DNA site. Thus, since these FACS or MACS-assisted methods do not allow the immediate enrichment of edited cells, genome editing in particular in hard-to-transfect cells (e.g. human primary cell lines) remains still excessively difficult.

A genome co-editing approach for the enrichment of CRISPR-edited cells involving the genome editing of a gene of interest and LRRC8 genes along with the use of blasticidin as a selection marker is described in WO 2019/202099. A related approach of co-editing using 6-thioguanine (6TG) as the selection agent and the knock-out of the (hypoxanthine phosphoribosyltransferase) HPRT (encoding gene the hypoxanthine phosphoribosyltransferase) to render cells 6TG-resistant is described in Liao et al, Nucleic Acids Res. 2015; 43(20):e134. While these approaches efficiently enrich genome-edited cells they are based on the co-editing of a second gene being and selection marker.

The present invention therefore aims at providing technically improved methods and means which enable the enrichment of CRISPR-edited cells (step 3, above). As a yet further advantage the present invention enables the genomic target-specific depletion of cells within a population of cells by a CRISPR nuclease.

Accordingly, the present invention relates in a first aspect to a method for the genome editing of cells at a target locus by homology directed repair (HDR) within a cell population and concurrently enriching within said cell population the HDR-genome edited cells, wherein the method comprises (A) introducing into the cells within the cell population one or more nucleic acid molecules, said one or more nucleic acid molecules encoding in expressible form (i) a CRISPR nuclease comprising of consisting of (a) an amino acid sequence of any one of SEQ ID NO: 1, 2 or 3; (b) an amino acid sequence being encoded by the nucleotide sequence of SEQ ID NO: 4, 5, 6 or 7; (c) an amino acid sequence being at least 80% identical to the amino acid sequence of (a), or (d) an amino acid sequence being encoded by a nucleic acid sequence being at least 80% identical to the nucleotide sequence of (b), (ii) a guide RNA comprising (a) a first segment comprising a nucleotide sequence that is complementary to a sequence at the target locus; and (a) a second segment that interacts with the CRISPR nuclease of (i), and a nucleic acid molecule comprising, consisting of, or encoding (iii) a donor template with homology to the target locus; or (A′) introducing into the cells within the cell population (i) a CRISPR nuclease comprising or consisting of (a) an amino acid sequence of any one of SEQ ID NO: 1, 2 or 3; (b) an amino acid sequence being encoded by the nucleotide sequence of SEQ ID NO: 4, 5, 6 or 7; (c) an amino acid sequence being at least 80% identical to the amino acid sequence of (a), or (d) an amino acid sequence being encoded by a nucleic acid sequence being at least 80% identical to the nucleotide sequence of (b), a nucleic acid molecule, said nucleic acid molecule encoding in expressible form (ii) a guide RNA comprising (a) a first segment comprising a nucleotide sequence that is complementary to a sequence at the target locus; and (a) a second segment that interacts with the CRISPR nuclease of (i), and a nucleic acid molecule comprising, consisting of, or encoding (iii) a donor template with homology to the target locus; or (A″) introducing into the cells within the cell population (i) a ribonucleoprotein complex (RNP) comprising or consisting of a CRISPR nuclease comprising or consisting of (a) an amino acid sequence of any one of SEQ ID NO: 1, 2 or 3; (b) an amino acid sequence being encoded by the nucleotide sequence of SEQ ID NO: 4, 5, 6 or 7; (c) an amino acid sequence being at least 80% identical to the amino acid sequence of (a), or (d) an amino acid sequence being encoded by a nucleic acid sequence being at least 80% identical to the nucleotide sequence of (b), in complex with a guide RNA comprising (a) a first segment comprising a nucleotide sequence that is complementary to a sequence at the target locus; and (a) a second segment that interacts with the CRISPR nuclease (ii) a nucleic acid molecule comprising, consisting of, or encoding a donor template with homology to the target locus; and (B) culturing the cells under conditions wherein the target locus with the cells of the cell population becomes genome edited by homology directed repair (HDR) and the CRISPR nuclease concurrently enriches within said cell population the HDR-genome edited cells.

A cell population designates a group of cells. The cell population may be heterogeneous or homogeneous and is preferably homogeneous. A heterogeneous cell population comprises cells of different origin, e.g. from different species or sources and/or different cell-types of one species or source (e.g. body site). By contrast, a homogeneous cell population only comprises cells from one species or source and preferably only cells of one cell-type or one body site.

Genome editing (also known as genome engineering) is a type of genetic engineering in which a gene of interest is inserted, deleted, modified or replaced in the genome of the cell. In accordance with the first aspect of the invention, the genome editing employs the homology directed recombination (HDR) pathway. As compared to non-homologous end-joining (NHEJ) and HDR is the more accurate mechanism for DSB repair due to the requirement of higher sequence homology between the damaged and intact donor strands of DNA. The process is error-free if the DNA template used for repair is identical to the original DNA sequence at the DSB, or it can introduce specific mutations into the damaged DNA. It is therefore to be understood that cells in the population of cells harboring the target locus to be genome edited are capable of HDR.

Genome editing may result in a loss-of-function mutation or a gain-of-function mutation in the genome of the cell. A loss-of-function mutation (also called inactivating mutation) results in the gene of interest having less or no function (being partially or wholly inactivated). When the allele has a complete loss of function (wholly inactivated) this is also called herein a (gene) knock-out. Genome editing of the gene of interest is preferably a knock-out. A gene knock-out may be achieved by inserting, deleting, modifying or replacing one or more nucleotides of a gene. A gain-of-function mutation (also called activating mutation) may change the gene of interest such that its effect gets stronger (enhanced activation) or even is superseded by a different (e.g. abnormal) function. A gain-of-function mutation may also introduce a new function or effect into a cell which the cell did not have before. In this context the new gene may be added to the genome of the cell (insertion) or may replace a gene within the genome. A gain-of-function mutation introducing such a new function or effect is also called gene knock-in.

Genome editing via HDR in accordance with the first aspect of the invention relies on (i) a nuclease of the clustered regularly interspaced short palindromic repeats (CRISPR/Cas) system which is also designated “CRISPR nuclease” herein, (ii) a guide RNA that mediates the interaction between CRISPR nucleases and the target locus which is also referred to herein as “spacer/protospacer” (noting that the spacer binds to the target locus while the protospacer (or protospacer adjacent motif (PAM)) binds to the CRISPR nuclease), and (iii) a donor template with homology to the target locus which is also referred to herein as “HDR template”.

CRISPR nucleases (or CRISPR-Cas nucleases or Cas nucleases) are a specific type of programmable nucleases. The CRISPR nucleases to be used in accordance with the present invention are so-called BRAIN Engineered Cas proteins (BEC) nucleases. In more detail, the amino acid sequence of SEQ ID NO: 1, 2 or 3 and the nucleotide sequence of SEQ ID NO: 4, 5 or 6 are the amino acid sequences and the nucleotide sequences of the class 2, type V RNA-guided DNA nucleases of BEC85, BEC67 and BEC10 as disclosed in the international application PCT/EP2021/000081. Among the three BEC nucleases BEC10 is most preferred. SEQ ID NO: 7 is BEC10 that has been optimized for the expression in E. coli. SEQ ID NO: 7 can therefore also be designated E. coli BEC10.

The CRISPR-Cas system has been harnessed for genome editing in prokaryotes and eukaryotes. A small piece of RNA with a short “guide” sequence that attaches (binds) to a specific target sequence of DNA in a genome is created (the so-called guide RNA (gRNA) or single guide (sgRNA)). The genomic target site of the gRNA can be any ˜20 nucleotide DNA sequence, provided it meets two conditions: (i) The sequence is unique compared to the rest of the genome, and (ii) the target is present immediately adjacent to a protospacer adjacent motif (PAM). The PAM sequence is essential for target binding, but the exact sequence depends on which CRISPR nuclease is used. CRISPR nucleases and their respective PAM sequences are known in the art (see https://www.addgene.org/crispr/guide/#pam-table). Hence, the gRNA also binds to the CRISPR nuclease (the BEC enzyme). As in bacteria, the gRNA is used to recognize the DNA sequence, and the CRISPR nuclease cuts the DNA at the targeted location. Once the DNA is cut, the cell's own DNA repair machinery (NHEJ or HDR) adds or deletes pieces of genetic material, or makes changes to the DNA by replacing an existing segment with a customized DNA sequence. Hence, in the CRISPR-Cas system, the CRISPR nuclease makes a double-stranded break in DNA at a site determined by the short (˜20 nucleotide) gRNA which break is then repaired within the cell by NHEJ or HDR. The CRISPR-Cas system can be multiplexed by adding multiple gRNAs. It was demonstrated that, for example, five different simultaneous mutations can be introduced into mouse embryonic stem cells by using five different gRNA molecules and one CRISPR nuclease.

The designs and structures of donor templates being suitable for HDR are known in the art. HDR is error-free if the repair template is identical to the original DNA sequence at the double-strand break (DSB), or it can introduce very specific mutations into DNA. The three central steps of the HDR pathways are: (1) The 5′-ended DNA strand is resected at the break to create a 3′ overhang. This will serve as both a substrate for proteins required for strand invasion and a primer for DNA repair synthesis. (2) The invasive strand can then displace one strand of the homologous DNA duplex and pair with the other. This results in the formation of the hybrid DNA, referred to as the displacement loop (D loop). (3) The recombination intermediates can then be resolved to complete the DNA repair process.

HDR templates used, for example, to introduce mutations or insert new nucleotides or nucleotide sequences into a gene require a certain amount of homology surrounding the target sequence that will be modified. Homology arms can be used that start at the CRISPR-induced DSB. In general, the insertion sites of the modification should be very close to the DSB, ideally less than 10 bp away, if possible. One important point to note is that the CRISPR enzymes may continue to cleave DNA once a DSB is introduced and repaired. As long as the gRNA target site/PAM site remain intact, the CRISPR nuclease will keep cutting and repairing the DNA. This repeated editing may be problematic if a very specific mutation or sequence is to be introduced into a gene of interest. To get around this, the repair template can be designed in such a way that it will ultimately block further CRISPR nuclease targeting after the initial DSB is repaired. Two common ways to block further editing are mutating the PAM sequence or the gRNA seed sequence. When designing a repair template, the size of the intended edit is to be taken into consideration. ssDNA templates (also referred to as ssODNs) are commonly used for smaller modifications. Small insertions/edits may require as little as 30-50 bases for each homology arm, and the best exact number may vary based on the gene of interest. 50-80 base homology arms are commonly used. For example, Richardson et al. (Nat Biotechnol. 2016 March; 34(3):339-44) found that asymmetric homology arms (36 bases distal to the PAM and 91 bases proximal to the PAM) supported HDR efficiencies up to 60%. Due to difficulties that might be associated with creating ssODNs longer than 200 bases, it is preferred to use dsDNA plasmid repair templates for larger insertions such as fluorescent proteins or selection cassettes into a gene of interest. These templates can have homology arms of at least 800 bp. To increase the frequency of HDR edits based on plasmid repair templates, self-cleaving plasmids can be used that contain gRNA target sites flanking the template. When the CRISPR nuclease and the appropriate gRNA(s) are present, the template is liberated from the vector. To avoid plasmid cloning, it is possible to use PCR-generated long dsDNA templates. Moreover, Quadros et al. (Genome Biol. 2017 May 17; 18(1):92) developed Easi-CRISPR, a technique that allows making large mutations and to take advantage of the benefits of ssODNs. To create ssODNs longer than 200 bases, RNA encoding the repair template are in vitro transcribed and then reverse transcriptase is used to create the complementary ssDNA. Easi-CRISPR works well in mouse knock-in models, increasing editing efficiency from 1-10% with dsDNA to 25-50% with ssODNs. Although HDR efficiency varies across loci and experimental systems, ssODN templates generally provide the highest frequency of HDR edits.

The target site is not particularly limited and refers to a genomic site of interest that is present in the genome of cells to be genome edited. The target site in the genome designates mitochondrial DNA or genomic DNA and preferably genomic DNA. The target site is preferably but not necessarily a gene of interest (or target gene). The target site may also be, for example, a gene regulatory element, such as a promoter region or a cis-regulatory element.

In accordance with step (A) of the method of the invention one or more nucleic acid molecules encoding in expressible form the CRISPR nuclease as well as the guide RNA, and in addition the HDR template either directly (in the form a single-stranded or double-stranded DNA) or in expressible form encoded by a nucleic acid molecule (preferably an expression vector) are introduced into the cells within the cell population, whereas in accordance with step (A′) of the method of the invention the CRISPR nuclease itself (i.e. in proteinaceous form), a nucleic acid molecule encoding the guide RNA, and in addition the HDR template either directly or in expressible form encoded by a nucleic acid molecule are introduced into the cells within the cell population. Also in accordance with step (A″) the CRISPR nuclease itself (i.e. in proteinaceous form) is introduced into the cells within the cell population, however, in this case in the form of a ribonucleoprotein complex (RNP) together with a guide RNA. RNPs are assembled in vitro and can be delivered to the cell by methods known in the art, for example, electroporation or lipofection. RNPs are capable of cleaving the target site with comparable efficacy as nucleic acid-based (e.g. vector-based) CRISPR nucleases (Kim et al. (2014), Genome Research 24(6):1012-1019). Also in accordance with step (A″) the HDR template is either directly introduced into the cells within the cell population or in an expressible form encoded by a nucleic acid molecule introduced into the cells within the cell population.

In the cases where the HDR template is in expressible form encoded by a nucleic acid molecule said nucleic acid molecule may be a separate nucleic acid molecule or the same nucleic acid molecule that encodes the CRISPR nuclease and/or the guide RNA. In the cases where the HDR template is directly introduced into the cell the HDR template may be a linear double-stranded or single-stranded DNA molecule. The linear double-stranded DNA molecule is preferably a linearized PCR product and the linear single-stranded DNA molecule is preferably a ssODN as described herein above.

The nucleic acid molecules used in accordance with of the present invention may be inserted into several commercially available vectors. Single vectors containing both the CRISPR nuclease and the gRNAs and optionally the HDR template are commercially available, thereby acting as an all-in-one vector. The method of the invention can alternatively be implemented by using two or three vectors containing the CRISPR nuclease, gRNA and the HDR template. It is also possible to use gRNA and/or HDR template-only vectors and to use cells in which the CRISPR nuclease has been integrated into the genome. The use of an all-in-one vector that expresses the gRNA, the CRISPR nuclease and optionally the HDR template is preferred since only one vector is to be introduced into the cells. A vector which can express the CRISPR nuclease and up to seven gRNAs is, for example, described in Sakuma et al, Sci Rep. 2014; 4: 5400.

Many single gRNA empty vectors (with and without the CRISPR nuclease) are available in the art. Likewise several empty multiplex gRNA vectors are available that can be used to express multiple gRNAs from a single plasmid (with or without the expression of the CRISPR nuclease).

Finally, also vectors are available that only express the CRISPR nuclease (see https://www.addgene.org/crispr/empty-grna-vectors/).

Vector modification techniques are known in the art and, for example, described in Sambrook and Russel, 2001. Generally, vectors can contain one or more origins of replication (ori) and inheritance systems for cloning or expression, one or more markers for selection in the host, e.g., antibiotic resistance, and one or more expression cassettes. Suitable origins of replication include, for example, the Col E1, the SV40 viral and the M 13 origins of replication. The nucleic acid sequences inserted in the vector can e.g. be synthesized by standard methods, or isolated from natural sources. Ligation of the coding sequences to transcriptional regulatory elements and/or to other amino acid encoding sequences can be carried out using established methods. Such regulatory sequences are well known to those skilled in the art and include, without being limiting, regulatory sequences ensuring the initiation of transcription, initiation of translation, internal ribosomal entry sites (IRES) or 2A linker (Owens, Proc. Natl. Acad. Sci. USA 98 (2001), 1471-1476) and optionally regulatory elements ensuring termination of transcription and stabilization of the transcript. Non-limiting examples for regulatory elements ensuring the initiation of transcription comprise a translation initiation codon, enhancers such as e.g. the SV40-enhancer, insulators and/or promoters, such as for example the cytomegalovirus (CMV) promoter, elongation factor-1 alpha (EF1-alpha), promoter, SV40-promoter, RSV-promoter (Rous sarcoma virus), the lacZ promoter, chicken beta-actin promoter, CAG-promoter (a combination of chicken beta-actin promoter and cytomegalovirus immediate-early enhancer), the gai10 promoter, human elongation factor 1a-promoter, AOX1 promoter, GAL1 promoter, CaM-kinase promoter, the lac, trp or tac promoter, the lacUV5 promoter, the Autographa californica multiple nuclear polyhedrosis virus (AcMNPV) polyhedral promoter or a globin intron in mammalian and other animal cells. Non-limiting examples for regulatory elements ensuring transcription termination include the V40-poly-A site, the tk-poly-A site or the SV40, lacZ or AcMNPV polyhedral polyadenylation signals, which are to be included downstream of the nucleic acid sequence of the invention. Additional regulatory elements may include translational enhancers, Kozak sequences and intervening sequences flanked by donor and acceptor sites for RNA splicing, nucleotide sequences encoding secretion signals or, depending on the expression system used, signal sequences capable of directing the expressed polypeptide to a cellular compartment. Moreover, elements such as origin of replication, drug resistance gene, regulators (as part of an inducible promoter) may also be included.

Means and methods for the introduction for the nucleic acid molecule(s) expressing the CRISPR nuclease and/or the gRNAs and optionally (further) the HDR template into cells are known in the art and these methods encompass transducing or transfecting cells.

Transduction is the process by which foreign DNA is introduced into a cell by a virus or viral vector. Transduction is a common tool used by molecular biologists to stably introduce a foreign gene into a host cell's genome. Generally, a plasmid is constructed in which the genes to be transferred are flanked by viral sequences that are used by viral proteins to recognize and package the viral genome into viral particles. This plasmid is inserted (usually by transfection) into a producer cell together with other plasmids (DNA constructs) that carry the viral genes required for formation of infectious virions. In these producer cells, the viral proteins expressed by these packaging constructs bind the sequences on the DNA/RNA (depending on the type of viral vector) to be transferred and inserted into viral particles. For safety, none of the plasmids used contains all the sequences required for virus formation, so that simultaneous transfection of multiple plasmids is required to get infectious virions. Moreover, only the plasmid carrying the sequences to be transferred contains signals that allow the genetic materials to be packaged in virions, so that none of the genes encoding viral proteins are packaged. Viruses collected from these cells are then applied to the cells to be altered. The initial stages of these infections mimic infection with natural viruses and lead to expression of the genes transferred and (in the case of lentivirus/retrovirus vectors) insertion of the DNA to be transferred into the cellular genome. However, since the transferred genetic material does not encode any of the viral genes, these infections do not generate new viruses (the viruses are “replication-deficient”). In the present case transduction may be used to generate cells that comprise the CRISPR nuclease in their genome in expressible form.

Transfection is the process of deliberately introducing naked or purified nucleic acids or purified proteins or assembled ribonucleoprotein complexes into cells. Transfection is generally a non-viral based method.

Transfection may be a chemical-based transfection. Chemical-based transfection can be divided into several kinds: transfection using cyclodextrin, polymers, liposomes, or nanoparticles. One of the cheapest methods uses calcium phosphate. HEPES-buffered saline solution (HeBS) containing phosphate ions are combined with a calcium chloride solution containing the DNA to be transfected. When the two are combined, a fine precipitate of the positively charged calcium and the negatively charged phosphate will form, binding the DNA to be transfected on its surface. The suspension of the precipitate is then added to the cells to be transfected (usually a cell culture grown in a monolayer). By a process not entirely understood, the cells take up some of the precipitate, and with it, the DNA. This process has been a preferred method of identifying many oncogenes. Other methods use highly branched organic compounds, so-called dendrimers, to bind the DNA and transfer it into the cell. Another method is the use of cationic polymers such as DEAE-dextran or polyethylenimine (PEI). The negatively charged DNA binds to the polycation and the complex is taken up by the cell via endocytosis. Lipofection (or liposome transfection) is a technique used to inject genetic material into a cell by means of liposomes, which are vesicles that can easily merge with the cell membrane since they are both made of a phospholipid bilayer. Lipofection generally uses a positively charged (cationic) lipid (cationic liposomes or mixtures) to form an aggregate with the negatively charged (anionic) genetic material. This transfection technology performs the same tasks in terms of transfer into cells as other biochemical procedures utilizing polymers, DEAE-dextran, calcium phosphate, and electroporation. The efficiency of lipofection can be improved by treating transfected cells with a mild heat shock. Fugene is a series of widely used proprietary non-liposomal transfection reagents capable of directly transfecting a wide variety of cells with high efficiency and low toxicity.

Transfection may also be a non-chemical method. Electroporation (gene electrotransfer) is a popular method, where transient increase in the permeability of cell membrane is achieved when the cells are exposed to short pulses of an intense electric field. Cell squeezing enables delivery of molecules into cells via cell membrane deformation. Sonoporation uses high-intensity ultrasound to induce pore formation in cell membranes. This pore formation is attributed mainly to the cavitation of gas bubbles interacting with nearby cell membranes since it is enhanced by the addition of ultrasound contrast agent, a source of cavitation nuclei. Optical transfection is a method where a tiny (˜1 μm diameter) hole is transiently generated in the plasma membrane of a cell using a highly focused laser. Protoplast fusion is a technique in which transformed bacterial cells are treated with lysozyme in order to remove the cell wall. Following this, fusogenic agents (e.g., Sendai virus, PEG, electroporation) are used in order to fuse the protoplast carrying the gene of interest with the recipient target cell.

Finally, transfection may be a particle-based method. A direct approach to transfection is the gene gun, where the DNA is coupled to a nanoparticle of an inert solid (commonly gold), which is then “shot” (or particle bombardment) directly into the target cell's nucleus. Hence, the nucleic acid is delivered through membrane penetration at a high velocity, usually connected to microprojectiles. Magnetofection, or magnet-assisted transfection, is a transfection method that uses magnetic force to deliver DNA into target cells. Impalefection is carried out by impaling cells by elongated nanostructures and arrays of such nanostructures such as carbon nanofibers or silicon nanowires which have been functionalized with plasmid DNA.

Means for introducing proteins (or peptides) into living cells are known in the art and comprise but are not limited to microinjection, electroporation, lipofection (using liposomes), nanoparticle-based delivery, and protein transduction. Any one of these methods may be used in connection with step (a′). In this regard, the CRISPR nuclease to be introduced may either be isolated from their natural environment or recombinantly produced.

A liposome used for lipofection is a small vesicle, composed of the same material as a cell membrane (i.e., normally a lipid bilayer e.g. made of phospholipids), which can be filled with one or more protein(s) (e.g. Torchilin V P. (2006), Adv Drug Deliv Rev., 58(14):1532-55). To deliver a protein into a cell, the lipid bilayer of the liposome can fuse with the lipid bilayer of the cell membrane, thereby delivering the contained protein into the cell. It is preferred that the liposomes used in accordance with invention are composed of cationic lipids. The cationic liposome strategy has been applied successfully to protein delivery (Zelphati et al. (2001). J. Biol. Chem. 276, 35103-35110). As known in the art, the exact composition and/or mixture of cationic lipids used can be altered, depending upon the protein(s) of interest and the cell type used (Felgner et al. (1994). J. Biol. Chem. 269, 2550-2561). Nanoparticle-based delivery of Cas9 ribonucleoprotein and donor DNA for the induction of homology-directed DNA repair is, for example, described in Lee et al. (2017), Nature Biomedical Engineering, 1:889-90.

Protein transduction specifies the internalisation of proteins into the cell from the external environment (Ford et al (2001), Gene Therapy, 8:1-4). This method relies on the inherent property of a small number of proteins and peptides (preferably 10 to 16 amino acids long) being able to penetrate the cell membrane. The transducing property of these molecules can be conferred upon proteins which are expressed as fusions with them and thus offer, for example, an alternative to gene therapy for the delivery of therapeutic proteins into target cells. Commonly used proteins or peptides being able to penetrate the cell membrane are, for example; the antennapedia peptide, the herpes simplex virus VP22 protein, HIV TAT protein transduction domain, peptides derived from neurotransmitters or hormones, or a 9×Arg-tag.

Microinjection and electroporation are well known in the art and the skilled person knows how to perform these methods. Microinjection refers to the process of using a glass micropipette to introduce substances at a microscopic or borderline macroscopic level into a single living cell. Electroporation is a significant increase in the electrical conductivity and permeability of the cell plasma membrane caused by an externally applied electrical field. By increasing permeability, protein (or peptides or nucleic acid sequences) can be introduced into the living cell.

The CRISPR nuclease may be introduced into the cells as an active enzyme or as a proenzyme. In the latter case the CRISPR nuclease is biochemically changed within the cells (for example by a hydrolysis reaction revealing the active site, or changing the configuration to reveal the active site), so that the proenzyme becomes an active enzyme.

The term “in expressible form” means that the one or more nucleic acid molecules may encode their constituents in a form that ensures that the guide RNAs and/or the HDR template (if being encoded) are transcribed and that the CRISPR nuclease (if being encoded) is transcribed and translated into the active enzyme in the cells.

Example 3 illustrates the method of the first aspect of the invention. Here, the BEC10 nuclease according to the invention and the prior art nuclease spCas9 are used in comparison in a method for the genome editing of P. pastoris cells at a target locus by HDR, noting that P. pastoris cells are capable of repairing dsDNA breaks by NHEJ. The result with the prior art nuclease spCas9 was as expected. The result was a mixture of cells wherein the target locus was successfully genome edited by HDR and cells wherein the target locus after the dsDNA break as introduced by spCas9 at the target locus was repaired by NHEJ instead of the incorporation of the HDR template. On the other hand, the result with the nuclease according to the invention BEC10 was totally unexpected and technically advantageous. This resulted in cells wherein the target locus was successfully genome edited HDR as desired in all 20 obtained clones. No clones that displayed repair of the dsDNA break by NHEJ were obtained. Hence, the method according to the first aspect of the invention provides for a significant increase with respect to the efficiency of obtaining cells that are genome edited by HDR. In Example 3 the efficiency was 100%.

The discussed results of Example 3 together with further results in Examples 2, 3 and 5 that will discussed herein below in connection with the second aspect of the invention demonstrate that the BEC nucleases display a novel mode of action that is to the best knowledge of the inventors not known from any prior art CRISPR nucleases. This novel mode of action enables and forms the basis of the various embodiments as described herein.

This novel mode of action can be described as “double-stranded DNA collateral cleavage activity”. It is known from the prior art that certain CRISPR nucleases can display single-stranded DNA collateral cleavage activity (ssDNA; Cas 12) or single-stranded RNA collateral cleavage activity (ssRNA; Cas13); see Shashital (2018), Genome medicine; 10:32. These prior art CRISPR nucleases bind via a guide RNA to their target locus leading to collateral ssDNA or ssRNA collateral activity. However, as the genome of almost all organisms consists of dsDNA those prior art CRISPR nucleases are not able to target genomic DNA and are not suitable for genome editing.

The results in Example 2, 3 and 5 indicate that the BEC nucleases according to the invention display double-stranded DNA collateral cleavage activity. Hence, essentially all dsDNA within the cell the genome of which is to be edited can be cleaved by the BEC nucleases once they become activated by binding to their target locus. To the best knowledge of the inventors the present disclosure is the first report of CRISPR nucleases with double-stranded DNA collateral cleavage activity.

It is therefore preferred that the CRISPR nucleases to be used herein are not only structurally defined by displaying the required sequence homology to the exact sequences of the BEC nucleases but in addition functionally defined as displaying double-stranded DNA collateral cleavage activity and/or as being capable of depleting cells that repaired the dsDNA break as introduced by the active BEC nucleases at the target locus by NHEJ.

In connection with the method of the first aspect of the invention it is of note that the cells wherein the target locus was successfully genome edited by HDR no longer display the target locus since the target locus was replaced by the donor template. These cells are protected from the dsDNA collateral cleavage activity. On the other hand, if the target locus is not eliminated by the introduction of the HDR template the BEC nuclease is activated by the matching spacer/protospacer sequences and the dsDNA collateral cleavage is induced. The dsDNA breaks in genomic DNA provided in trans are expected to deplete undesired cells without an introduction of the HDR template, and because of the collateral activity the intrinsic NHEJ repair mechanism of the cell is not able to prevent cell death. The unspecific dsDNA breaks in the genome kills the cells.

In accordance with a preferred embodiment of the first aspect of the invention the method further comprises (C) isolating one or more cells, wherein the target locus has been genome edited by homology directed repair (HDR).

It is emphasized that in Example 3 20 of 20 clones and, thus, 100% of the clones are comprised of cells, wherein the target locus has been genome edited by HDR and not by NHEJ. Hence, in the case of the method of the first aspect of the invention step (C) may just be as easy as the collecting of one or more cells from a culture plate.

In general means and methods for an isolation step from a heterogeneous population of cells are known in the art. Non-limiting examples are single-cell dilution, laser capture microdissection, manual or automated cell picking, FACS and MACS.

In single-cell dilution a solution comprising cells is diluted in more or more steps until a solution with only a single cell is obtained. Laser capture microdissection is a method for isolating specific cells of interest from microscopic regions of tissue, cells or organisms. A laser is coupled into a microscope and focuses onto on a selected cell within a cell population. By movement of the laser by optics or the stage the focus follows a trajectory which is predefined by the user. This trajectory with the selected cell, also called element, is then cut out and separated from the adjacent cells. Manual cell picking is a simple, convenient, and efficient method for isolating single cells. Manual cell picking micromanipulators consist of an inverted microscope combined with micro-pipettes that are movable through motorized mechanical stages. Cell picking can also be implemented into an automated device. Fluorescence Activated Cell Sorting (FACS), a specialized type of flow cytometry with sorting capacity, is the most sophisticated and user-friendly technique for characterizing and defining different cell types in a heterogeneous cell population based on size, granularity, and fluorescence. FACS allows simultaneous quantitative and qualitative multi-parametric analyses of single cells. Magnetic-Activated Cell Sorting (MACS) is another commonly used passive separation technique to isolate different types of cells depending on their cluster of differentiation. It has been reported that MACS is capable of isolating specific cell populations with a purity >90% purification.

The invention also pertains to an isolated cell obtained by the above method as well as a composition comprising this cell, wherein the composition is preferably an industrial composition, a diagnostic composition or a pharmaceutical composition. The pharmaceutical composition is preferred.

An industrial composition is intended to be used in industry, including agriculture. For instance, cells wherein a particular enzyme as the gene of interest has been introduced may be used in chemical production, biofuels, food & beverage, animal feeds, cosmetic products and consumer products.

A diagnostic composition is intended to be used in the diagnosis or a disease or condition. For instance, cells wherein a particular fluorescent protein as the gene of interest has been introduced may be used in diagnosis since they can be detected within an organism or a tissue sample.

The term “pharmaceutical composition” relates to a composition for administration to a patient, preferably a human patient. The pharmaceutical composition of the invention comprises the cells recited above. It may, optionally, comprise further molecules capable of altering the characteristics of the cells of the invention thereby, for example, stabilizing, modulating and/or activating their function. The composition is preferably in liquid form, e.g. (a) solution(s). The pharmaceutical composition of the present invention may, optionally and additionally, comprise a pharmaceutically acceptable carrier. Examples of suitable pharmaceutical carriers are well known in the art and include phosphate buffered saline solutions, water, emulsions, such as oil/water emulsions, various types of wetting agents, sterile solutions, organic solvents etc. Compositions comprising such carriers can be formulated by well-known conventional methods. These pharmaceutical compositions can be administered to the subject at a suitable dose. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. The therapeutically effective amount for a given situation will readily be determined by routine experimentation and is within the skills and judgement of the ordinary clinician or physician. Generally, the regimen as a regular administration of the pharmaceutical composition should be in the range of 1×10⁴ to 1×10⁸ cells per day. However, a more preferred dosage might be in the range of 1×10⁵ to 1×10⁷ cells and most preferably 5×10⁵ to 5×10⁶ cells per day.

The length of treatment needed to observe changes and the interval following treatment for responses to occur vary depending on the desired effect. The particular amounts may be determined by conventional tests which are well known to the person skilled in the art.

In accordance with preferred embodiments of the first aspect of the invention the donor template (i) inserts one or more nucleotides at the target locus, (ii) deletes one or more nucleotides at the target locus, and/or (ii) substitutes one or more nucleotides at the target locus.

This can be achieved by designing the donor template accordingly. As discussed above, the donor template generally comprises homology arms that ensure that the donor template can replace the target locus by HDR. The sequence between the homology arms can comprise additional nucleotides or stretch(es) of nucleotide(s) as compared to the target site (insertion); can lack can one or more nucleotides or stretch(es) of nucleotide(s) as compared to the target site (deletion), and/or can comprise one or more different nucleotides or different stretch(es) of nucleotide(s) as compared to the target site (substitution). The stretch of nucleotides of an insertion can comprise or consist of one or more genes, optionally together with one or more expression regulatory sequences, such as a promoter.

Hence, HDR template can be used to specifically modify a target locus, as needed, by the addition, deletion and/or substitution one or more nucleotides or stretch(es) of nucleotide(s).

In accordance with a further preferred embodiment of the first aspect of the invention the donor template comprises or consists of a nucleotide sequence with one or more intended mutations flanked by nucleotide sequences being homologous to the target locus.

The nucleotide sequences being homologous to the target locus are also referred to herein as homology arms. In the case of a double-stranded donor template the homology arms are preferably 30 to 800 bp, more preferably 300 to 500 bp. In the case of a single-stranded donor template the homology arms are preferably at least 300 bp and more preferably at least 800 bp.

As discussed, the sequence with one or more intended mutations between the arms can comprise the addition, deletion and/or substitution one or more nucleotides or stretch(es) of nucleotide(s).

In accordance with another preferred embodiment of the first aspect of the invention, the method further comprises synchronizing and capturing cells at the S and G2 phases in step (B).

The step of synchronizing and capturing cells at the S and G2 phases in step (B) can increase the efficiency of HDR-mediated genome editing; see, for example, Lin et al. (2014), eLife; 3: e04766. The synchronization and capturing of the cells at the S phase is preferred.

Cell cycle synchronization techniques are well-established and are reviewed, for example, in Jackman and O'Connor (2011), Curr Protoc Cell Biol; Chapter 8: Unit 8.3.

The present invention relates in a second aspect to a method for the selective depletion of cells comprising a target locus within a cell population, wherein the method comprises (A) introducing into the cells within the cell population one or more nucleic acid molecules, said one or more nucleic acid molecules encoding in expressible form (i) a CRISPR nuclease comprising or consisting of (a) an amino acid sequence of any one of SEQ ID NO: 1, 2 or 3; (b) an amino acid sequence being encoded by the nucleotide sequence of SEQ ID NO: 4, 5, 6 or 7; (c) an amino acid sequence being at least 80% identical to the amino acid sequence of (a), or (d) an amino acid sequence being encoded by a nucleic acid sequence being at least 80% identical to the nucleotide sequence of (b), and (ii) a guide RNA comprising (a) a first segment comprising a nucleotide sequence that is complementary to a sequence at the target locus and (b) a second segment that interacts with the CRISPR nuclease of (i); or (A′) introducing into the cells within the cell population (i) a CRISPR nuclease comprising or consisting of (a) an amino acid sequence of any one of SEQ ID NO: 1, 2 or 3; (b) an amino acid sequence being encoded by the nucleotide sequence of SEQ ID NO: 4, 5, 6 or 7; (c) an amino acid sequence being at least 80% identical to the amino acid sequence of (a), or (d) an amino acid sequence being encoded by a nucleic acid sequence being at least 80% identical to the nucleotide sequence of (b), and one or more nucleic acid molecules, said one or more nucleic acid molecules encoding in expressible form (ii) a guide RNA comprising (a) a first segment comprising a nucleotide sequence that is complementary to a sequence at the target locus and (b) a second segment that interacts with the CRISPR nuclease of (i); or (A″) introducing into the cells within the cell population (i) a ribonucleoprotein complex (RNP) comprising or consisting of (a) an amino acid sequence of any one of SEQ ID NO: 1, 2 or 3; (b) an amino acid sequence being encoded by the nucleotide sequence of SEQ ID NO: 4, 5, 6 or 7; (c) an amino acid sequence being at least 80% identical to the amino acid sequence of (a), or (d) an amino acid sequence being encoded by a nucleic acid sequence being at least 80% identical to the nucleotide sequence of (b), in complex with a guide RNA comprising (a) a first segment comprising a nucleotide sequence that is complementary to a sequence at the target locus and (b) a second segment that interacts with the CRISPR nuclease; and (B) culturing the cells under conditions wherein the CRISPR nuclease selectively depletes the cells within the cell population that comprise the target locus.

The definitions and preferred embodiments of the first aspect of the invention apply mutatis mutandis to the second aspect of the invention as far as being amendable for combination with the second aspect of the invention.

In this connection it is noted that the second aspect of the invention uses a CRISPR nuclease as well as guide RNA as defined in connection with the first aspect of the invention but does not use a donor template.

According to the second aspect of the invention the above-discussed novel mode of action of the BEC nucleases is used in order to selectively deplete cells comprising a particular target locus within a cell population. In Example 2 this is illustrated based on a guide RNA that targets an ampicillin resistance of vector DNA within E. coli cells, wherein the vector DNA also harbors a kanamycin resistance. E. coli cells cannot repair the dsDNA break as introduced into the ampicillin resistance without a HDR template since they are not capable of repairing double-stranded DNA breaks through NHEJ. The result with the prior art Cpf1 nucleases was as expected. The obtained E. coli cells were no longer resistant to ampicillin but remained resistant to kanamycin. The result with the BEC10 nuclease was as again unexpected and technically advantageous. The obtained E. coli cells were no longer resistant to ampicillin and kanamycin. These results are explained by the dsDNA collateral cleavage activity as discussed herein above which also add dsDNA breaks into the kanamycin resistance. The results of Example 2 are confirmed by the results in Example 3, wherein no donor template was used. Here no clones were obtained since the NHEJ-repaired cells were removed by the BEC10 nuclease and the generation of HDR-repaired cells is not possible in the absence of the donor template. Also Example 5 illustrates the method of the second aspect of the invention by showing that the BEC10 nuclease can be used to deplete mammalian cells selectively, i.e. in a target cell specific manner.

It is therefore also preferred herein that the CRISPR nucleases to be used herein are not only structurally defined by displaying the required sequence homology to the exact sequences of the BEC nucleases but in addition functionally defined as displaying double-stranded DNA collateral cleavage activity and/or as being capable of depleting cells comprising a target locus.

In accordance with a preferred embodiment of the first and second aspect of the invention, the cells within the population are capable of repairing double-stranded DNA breaks through non-homologous end joining (NHEJ).

As shown in Example 3 cells being capable of repairing double-stranded DNA breaks by NHEJ may repair the double-stranded DNA break as introduced by CRISPR nuclease by NHEJ. This is in particular undesired in case genome editing via a HDR template is desired. It is demonstrated that the BEC nucleases according to the present invention are capable of removing or preventing the occurrence of cells wherein the double-stranded DNA is repaired by NHEJ, in the presence of HDR template and of note also it its absence. It is believed that this is achieved by the above described dsDNA collateral activity of the BEC nucleases according to the present invention.

In accordance with a further preferred embodiment of the first and second aspect of the invention, the cells within the population are prokaryotic cells. Alternatively and also preferred, they are eukaryotic cells, and are more preferably vertebrate cells, and most preferably mammalian cells.

The prokaryotic cells are preferably bacterial cells, such as E. coli or B. subtilis cells.

Eukaryotic cells are preferred as compared to prokaryotic cells. The eukaryotic cells can not only be chordate or vertebrate cells but can also be yeast cells, such as a P. pastoris or A. niger cells.

The vertebrate cells are preferably cells of a vertebrate (in particular mammalian) cell line, organoids, primary cells, cells from a primary cell line, or pluripotent stem cells.

A mammalian cell line is a population of cells from a mammal which would normally not proliferate indefinitely but, due to mutation (that naturally occurred, e.g. in a tumor or by artificial mutagenesis), have evaded normal cellular senescence and instead can keep undergoing division. The cells can therefore be grown for prolonged periods in vitro.

An organoid is a miniaturized and simplified version of an organ produced in vitro in three dimensions that shows realistic micro-anatomy. They are derived from one or a few cells from a tissue, embryonic stem cells or induced pluripotent stem cells, which can self-organize in three-dimensional culture owing to their self-renewal and differentiation capacities.

Primary cells are cells taken directly from living tissue (e.g. biopsy material) and established for growth in vitro. These cells have undergone very few population doublings and are therefore more representative of the main functional component of the tissue from which they are derived in comparison to continuous (tumor or artificially immortalized) cell lines, thus generally representing a more representative model to the in vivo state. A primary cell line is a cell line that has been established from primary cells.

Pluripotent stem cells are cells that have the capacity to self-renew by dividing and to develop into the three primary germ cell layers of the early embryo and therefore into all cells of the adult body, but not extra-embryonic tissues such as the placenta. Embryonic stem cells and induced pluripotent stem cells are pluripotent stem cells. Embryonic stem cells are derived from the inner cell mass of a blastocyst, an early-stage pre-implantation embryo. Human embryos reach the blastocyst stage 4-5 days post fertilization, at which time they consist of 50-150 cells. They are preferably isolated from the embryo without the destruction of the embryo. Induced pluripotent stem cells (also known as iPS cells or iPSCs) are a type of pluripotent stem cell that can be generated directly from adult cells. The iPSC technology was pioneered by Shinya Yamanaka's lab in Kyoto, Japan, who showed in 2006 that the introduction of four specific genes encoding transcription factors could convert adult cells into pluripotent stem cells. The generation of iPSCs using Oct3/4 and/or a factor belonging to the Myc, Klf and Sox families of factors is described in WO 2009/144008.

In accordance with another preferred embodiment of the first and second aspect of the invention, the method is an in vitro or ex vivo method.

An ex vivo method is a method being carried out of the context of a living organism. Similarly, in vitro methods are performed with microorganisms, cells, or biological molecules outside their normal biological context.

Both, in vitro and ex vivo methods exclude methods for treatment of the human or animal body by surgery or therapy and diagnostic methods practised on the human or animal body.

The present invention relates in a third aspect to (A) one or more nucleic acid molecules, said one or more nucleic acid molecules encoding in expressible form (i) a CRISPR nuclease comprising or consisting of (a) an amino acid sequence of any one of SEQ ID NO: 1, 2 or 3; (b) an amino acid sequence being encoded by the nucleotide sequence of SEQ ID NO: 4, 5, 6 or 7; (c) an amino acid sequence being at least 80% identical to the amino acid sequence of (a), or (d) an amino acid sequence being encoded by a nucleic acid sequence being at least 80% identical to the nucleotide sequence of (b), and (ii) a guide RNA comprising (a) a first segment comprising a nucleotide sequence that is complementary to a sequence at the target locus; and (a) a second segment that interacts with the CRISPR nuclease of (i); or (A′) (i) a CRISPR nuclease comprising or consisting of (a) an amino acid sequence of any one of SEQ ID NO: 1, 2 or 3; (b) an amino acid sequence being encoded by the nucleotide sequence of SEQ ID NO: 4, 5, 6 or 7; (c) an amino acid sequence being at least 80% identical to the amino acid sequence of (a), or (d) an amino acid sequence being encoded by a nucleic acid sequence being at least 80% identical to the nucleotide sequence of (b), and one or more nucleic acid molecules, said one or more nucleic acid molecules encoding in expressible form (ii) a guide RNA comprising (a) a first segment comprising a nucleotide sequence that is complementary to a sequence at the target locus; and (a) a second segment that interacts with the CRISPR nuclease of (i); or (A″) a ribonucleoprotein complex (RNP) comprising or consisting of (a) an amino acid sequence of any one of SEQ ID NO: 1, 2 or 3; (b) an amino acid sequence being encoded by the nucleotide sequence of SEQ ID NO: 4, 5, 6 or 7; (c) an amino acid sequence being at least 80% identical to the amino acid sequence of (a), or (d) an amino acid sequence being encoded by a nucleic acid sequence being at least 80% identical to the nucleotide sequence of (b), in complex with a guide RNA comprising (a) a first segment comprising a nucleotide sequence that is complementary to a sequence at the target locus; and (a) a second segment that interacts with the CRISPR nuclease for use in treating a disease by the selectively depletion of cells comprising a target locus being associated with the disease to be treated.

Also described herein is a method of treating a disease by the selectively depletion of cells comprising a target locus being associated with the disease to be treated comprising administering to a subject in need thereof a therapeutically effective amount of (A) one or more nucleic acid molecules, said one or more nucleic acid molecules encoding in expressible form (i) a CRISPR nuclease comprising or consisting of (a) an amino acid sequence of any one of SEQ ID NO: 1, 2 or 3; (b) an amino acid sequence being encoded by the nucleotide sequence of SEQ ID NO: 4, 5, 6 or 7; (c) an amino acid sequence being at least 80% identical to the amino acid sequence of (a), or (d) an amino acid sequence being encoded by a nucleic acid sequence being at least 80% identical to the nucleotide sequence of (b), and (ii) a guide RNA comprising (a) a first segment comprising a nucleotide sequence that is complementary to a sequence at the target locus; and (a) a second segment that interacts with the CRISPR nuclease of (i); or (A′) (i) a CRISPR nuclease comprising or consisting of (a) an amino acid sequence of any one of SEQ ID NO: 1, 2 or 3; (b) an amino acid sequence being encoded by the nucleotide sequence of SEQ ID NO: 4, 5, 6 or 7; (c) an amino acid sequence being at least 80% identical to the amino acid sequence of (a), or (d) an amino acid sequence being encoded by a nucleic acid sequence being at least 80% identical to the nucleotide sequence of (b), and one or more nucleic acid molecules, said one or more nucleic acid molecules encoding in expressible form (ii) a guide RNA comprising (a) a first segment comprising a nucleotide sequence that is complementary to a sequence at the target locus; and (a) a second segment that interacts with the CRISPR nuclease of (i); or (A″) a ribonucleoprotein complex (RNP) comprising or consisting of (a) an amino acid sequence of any one of SEQ ID NO: 1, 2 or 3; (b) an amino acid sequence being encoded by the nucleotide sequence of SEQ ID NO: 4, 5, 6 or 7; (c) an amino acid sequence being at least 80% identical to the amino acid sequence of (a), or (d) an amino acid sequence being encoded by a nucleic acid sequence being at least 80% identical to the nucleotide sequence of (b), in complex with a guide RNA comprising (a) a first segment comprising a nucleotide sequence that is complementary to a sequence at the target locus; and (a) a second segment that interacts with the CRISPR nuclease.

The definitions and preferred embodiments of the first and second aspect of the invention apply mutatis mutandis to the third aspect of the invention as far as being amendable for combination with the third aspect of the invention.

The disease that benefits from the selective depletion of cells comprising a target locus being associated with the disease to be treated is generally a disease being a candidate for gene knockdown or knockout therapy.

The disease that benefits from the selective depletion of cells comprising a target locus being associated with the disease to be treated is preferably selected from the group consisting of cystic fibrosis, hemophilia A or hemophilia B with or without inhibitors, thalassemia, anemia, Alzheimer's disease, Parkinson's disease, Huntington's disease, amyotrophic lateral sclerosis (ALS), epilepsy, lysosomal storage diseases, Wilson's or Menkes disease, lysosomal acid lipase deficiency cancer, type 1 or type 2 diabetes, Gaucher's disease, Hurler's disease, adenosine deaminase deficiency, a glycogen storage disease), RPE65 deficiency, choroideremia, a viral infection, hepatitis B, hepatitis C, HIV, or a bacterial or fungal infection.

In a related aspect the present invention relates to (A) one or more nucleic acid molecules, said one or more nucleic acid molecules encoding in expressible form (i) a CRISPR nuclease comprising of consisting of (a) an amino acid sequence of any one of SEQ ID NO: 1, 2 or 3; (b) an amino acid sequence being encoded by the nucleotide sequence of SEQ ID NO: 4, 5, 6 or 7; (c) an amino acid sequence being at least 80% identical to the amino acid sequence of (a), or (d) an amino acid sequence being encoded by a nucleic acid sequence being at least 80% identical to the nucleotide sequence of (b), (ii) a guide RNA comprising (a) a first segment comprising a nucleotide sequence that is complementary to a sequence at the target locus and (b) a second segment that interacts with the CRISPR nuclease of (i), and a nucleic acid molecule comprising, consisting of, or encoding (iii) a donor template with homology to the target locus; or (A′) (i) a CRISPR nuclease comprising or consisting of (a) an amino acid sequence of any one of SEQ ID NO: 1, 2 or 3; (b) an amino acid sequence being encoded by the nucleotide sequence of SEQ ID NO: 4, 5, 6 or 7; (c) an amino acid sequence being at least 80% identical to the amino acid sequence of (a), or (d) an amino acid sequence being encoded by a nucleic acid sequence being at least 80% identical to the nucleotide sequence of (b), a nucleic acid molecule, said nucleic acid molecule encoding in expressible form (ii) a guide RNA comprising (a) a first segment comprising a nucleotide sequence that is complementary to a sequence at the target locus and (b) a second segment that interacts with the CRISPR nuclease of (i), and a nucleic acid molecule comprising, consisting of, or encoding (iii) a donor template with homology to the target locus; or (A″) (i) a ribonucleoprotein complex (RNP) comprising or consisting of a CRISPR nuclease comprising or consisting of (a) an amino acid sequence of any one of SEQ ID NO: 1, 2 or 3; (b) an amino acid sequence being encoded by the nucleotide sequence of SEQ ID NO: 4, 5, 6 or 7; (c) an amino acid sequence being at least 80% identical to the amino acid sequence of (a), or (d) an amino acid sequence being encoded by a nucleic acid sequence being at least 80% identical to the nucleotide sequence of (b), in complex with a guide RNA comprising (a) a first segment comprising a nucleotide sequence that is complementary to a sequence at the target locus and (b) a second segment that interacts with the CRISPR nuclease; and a nucleic acid molecule comprising, consisting of, or encoding (ii) a nucleic acid molecule encoding in expressible form a donor template with homology to the target locus for use in treating a disease by gene therapy.

Similarly, the present invention relates to a method for treating a disease by gene therapy by administering to a subject in need thereof a therapeutically effective amount of (A) one or more nucleic acid molecules, said one or more nucleic acid molecules encoding in expressible form (i) a CRISPR nuclease comprising of consisting of (a) an amino acid sequence of any one of SEQ ID NO: 1, 2 or 3; (b) an amino acid sequence being encoded by the nucleotide sequence of SEQ ID NO: 4, 5, 6 or 7; (c) an amino acid sequence being at least 80% identical to the amino acid sequence of (a), or (d) an amino acid sequence being encoded by a nucleic acid sequence being at least 80% identical to the nucleotide sequence of (b), (ii) a guide RNA comprising (a) a first segment comprising a nucleotide sequence that is complementary to a sequence at the target locus and (b) a second segment that interacts with the CRISPR nuclease of (i), and a nucleic acid molecule comprising, consisting of, or encoding (iii) a donor template with homology to the target locus; or (A′) (i) a CRISPR nuclease comprising or consisting of (a) an amino acid sequence of any one of SEQ ID NO: 1, 2 or 3; (b) an amino acid sequence being encoded by the nucleotide sequence of SEQ ID NO: 4, 5, 6 or 7; (c) an amino acid sequence being at least 80% identical to the amino acid sequence of (a), or (d) an amino acid sequence being encoded by a nucleic acid sequence being at least 80% identical to the nucleotide sequence of (b), a nucleic acid molecule, said nucleic acid molecule encoding in expressible form (ii) a guide RNA comprising (a) a first segment comprising a nucleotide sequence that is complementary to a sequence at the target locus and (b) a second segment that interacts with the CRISPR nuclease of (i), and a nucleic acid molecule comprising, consisting of, or encoding (iii) a donor template with homology to the target locus; or (A″) (i) a ribonucleoprotein complex (RNP) comprising or consisting of a CRISPR nuclease comprising or consisting of (a) an amino acid sequence of any one of SEQ ID NO: 1, 2 or 3; (b) an amino acid sequence being encoded by the nucleotide sequence of SEQ ID NO: 4, 5, 6 or 7; (c) an amino acid sequence being at least 80% identical to the amino acid sequence of (a), or (d) an amino acid sequence being encoded by a nucleic acid sequence being at least 80% identical to the nucleotide sequence of (b), in complex with a guide RNA comprising (a) a first segment comprising a nucleotide sequence that is complementary to a sequence at the target locus and (b) a second segment that interacts with the CRISPR nuclease; and a nucleic acid molecule comprising, consisting of, or encoding (ii) a nucleic acid molecule encoding in expressible form a donor template with homology to the target locus.

In gene therapy a mutated gene is replaced or a new gene is added into the genome of the subject in need thereof in in order to treat a disease. Gene therapy holds promise for treating a wide range of diseases. Non-limiting examples are cancer, cystic fibrosis, heart disease, diabetes, hemophilia and AIDS.

The desired therapeutic effect can be accomplished by the nature of the donor template. The donor template can be designed, for example, such that a mutated gene is replaced or repaired or by making cells more amenable for the attack of the immune system or a therapeutic antibody.

The present invention relates in a fourth aspect to a vector comprising in expressible form (i) a CRISPR nuclease comprising or consisting of (a) an amino acid sequence of any one of SEQ ID NO: 1, 2 or 3; (b) an amino acid sequence being encoded by the nucleotide sequence of SEQ ID NO: 4, 5, 6 or 7; (c) an amino acid sequence being at least 80% identical to the amino acid sequence of (a), or (d) an amino acid sequence being encoded by a nucleic acid sequence being at least 80% identical to the nucleotide sequence of (b), (ii) a guide RNA comprising (a) a first segment comprising a nucleotide sequence that is complementary to a sequence at the target locus; and (a) a second segment that interacts with the CRISPR nuclease of (i), and (iii) optionally a donor template with homology to the target locus.

The definitions and preferred embodiments of the first to third aspect of the invention apply mutatis mutandis to the fourth aspect of the invention as far as being amendable for combination with the fourth aspect of the invention.

The vector with a donor template is suitable for the method according to the first aspect of the invention while the vector without a donor template is suitable for the method according to the first and second aspect of the invention, noting that the donor template can also be supplied via a separate expression vector or as a nucleic acid molecule, such as a PCR product or a ssODN.

The vector or the CRSPR nuclease according to the invention may also be comprised in a kit. The kit preferably further comprises one or more of (i) a population of cells as defined herein, (ii) a medium for culturing these cells, and (iii) instructions for using the kit for genome editing of cells at a target locus by homology directed repair (HDR) within a cell population and concurrently enriching within said cell population the HDR-genome edited cells, and/or for the selective depletion of cells comprising a target locus within a cell population.

The components of the kit can be packed separately or in different combinations, taking into account the intended use for modifying a nucleotide sequence at a target site in the genome of a cell. The components of the kit can be packed, for example, into vials, tubes or bags.

The instructions can be in the format of a leaflet in the package or can also be in the format of a weblink, barcode or QR code on the package.

The present invention relates in a fifth aspect to the use of a CRISPR nuclease comprising or consisting of (a) an amino acid sequence of any one of SEQ ID NO: 1, 2 or 3; (b) an amino acid sequence being encoded by the nucleotide sequence of SEQ ID NO: 4, 5, 6 or 7; (c) an amino acid sequence being at least 80% identical to the amino acid sequence of (a), or (d) an amino acid sequence being encoded by a nucleic acid sequence being at least 80% identical to the nucleotide sequence of (b) for genome editing of cells at a target locus by homology directed repair (HDR) within a cell population and concurrently enriching within said cell population the HDR-genome edited cells, and/or for the selective depletion of cells comprising a target locus within a cell population.

The definitions and preferred embodiments of the first to fourth aspect of the invention apply mutatis mutandis to the fifth aspect of the invention as far as being amendable for combination with the fifth aspect of the invention.

This use is preferably an ex vivo or in vitro use.

As discussed herein above in connection with the first and second aspect of the invention for the use of the fifth aspect of the invention-the HDR-genome editing purpose and cell depletion use—the CRISPR nuclease according to the invention has to be used in combination with a guide RNA as defined herein above. For the HDR-genome editing use in addition a donor template as discussed herein above is to be used.

In accordance with a preferred embodiment of all aspects of the invention the sequence identity of at least 80% is preferably at least 85%, more preferably at least 90% and most preferably at least 95%.

The above at least 80% sequence identity with respect to these SEQ ID NOs is with increasing preference at least 80%, at least 85%, at least 90%, at least 91%, at least 92%, at least 93%, at least 94%, at least 95%, at least 96%, at least 98%, at least 99% and 100%.

Amino acid sequence as well as nucleotide sequence analysis and alignments in connection with the present invention are preferably carried out using the NCBI BLAST algorithm (Stephen F. Altschul, Thomas L. Madden, Alejandro A. Schaffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402). The skilled person is aware of additional suitable programs to align nucleic acid sequences.

Also in connection with this preferred embodiment it is preferred that the CRISPR nucleases to be used herein are not only structurally defined but are in addition functionally defined as displaying double-stranded DNA collateral cleavage activity. Alternatively or additionally the CRISPR nucleases may be defined as being capable of depleting cells that repaired the dsDNA break a introduce by the active BEC nucleases at the target locus by NHEJ (in cases where a HDR template is used), or as being capable of depleting cells comprising a target locus (in cases where no HDR template is used).

Regarding the embodiments characterized in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The figures show.

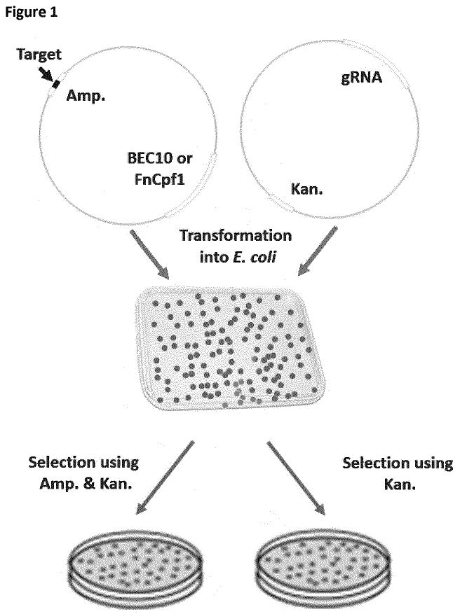

FIG. 1—Schematic figure visualizing the strategy to co-transfect two vectors into E. coli to compare the DNA targeting mechanism of the BEC10 nuclease in comparison to FnCpf1.

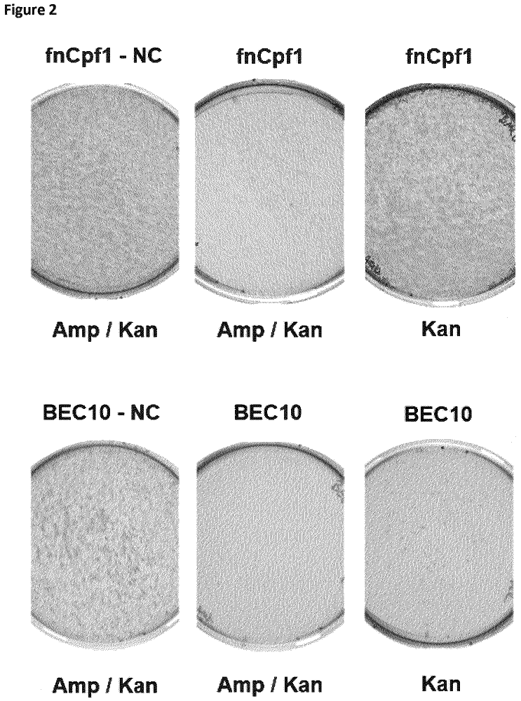

FIG. 2—Exemplary culture plates showing E. coli colonies 48 h after transformation to visualize the different molecular mechanism of BEC10 in comparison to FnCpf1.

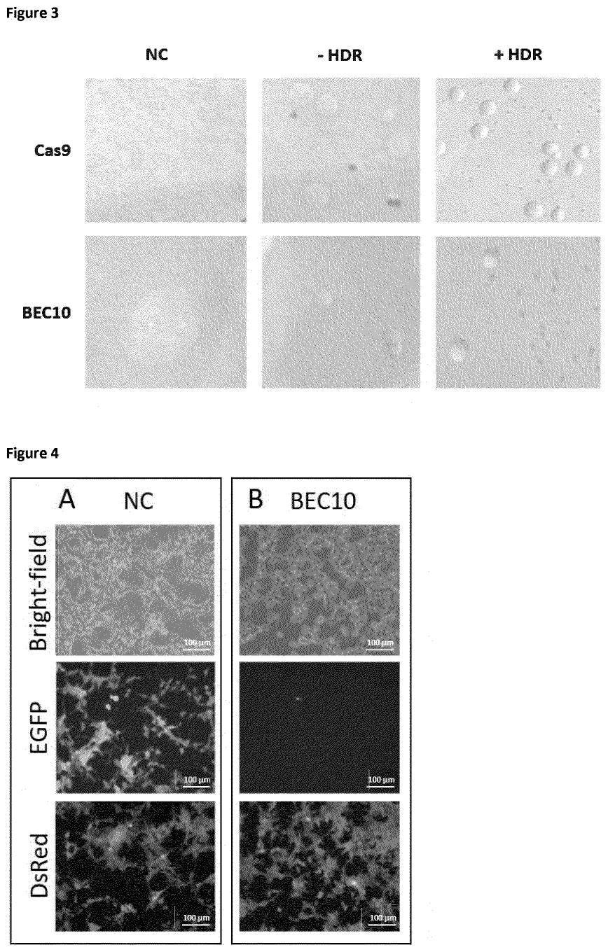

FIG. 3—Exemplary culture plates showing P. pastoris colonies 48 h after transformation to visualize the different genome editing mechanism of BEC10 in comparison to SpCas9.

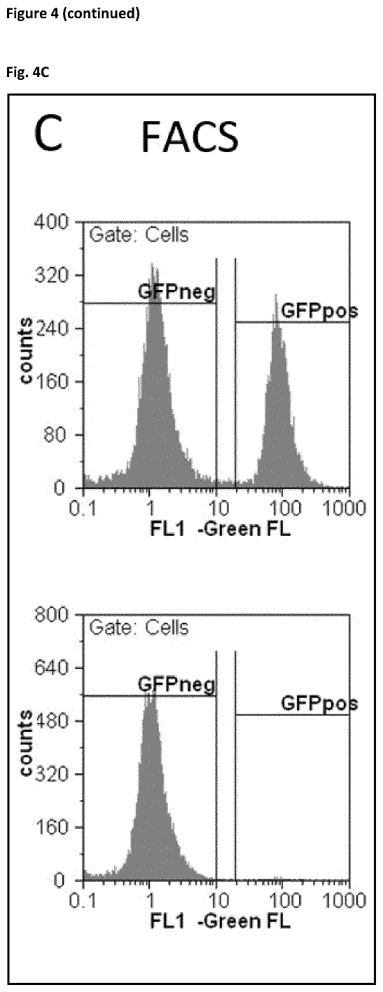

FIG. 4—Target specific cell depletion A: Negative control: 50/50 mixture of HEK-EGFP and HEK-DsRed cells transfected with BEC10 in combination with a non-targeting spacer B: Depletion of the EGFP positive cells using an EGFP specific spacer sequence to activate the BEC10 nuclease without affecting the viability of the EGFP negative (DsRed positive) cells. C: FACS analysis showing a distribution of ≈55% EGFP negative (left peak) and ≈45% EGFP positive cells (right peak) in the negative control (upper diagram) and more than 98.5% EGFP negative (left peak) and less than 1.5% EGFP positive cells (right peak) using a spacer matching the EGFP sequence (lower diagram).

The examples illustrate the invention.

### EXAMPLE 1

### Construction of a Functional Genome Editing System for E. coli (BEC10 and FnCpf1) and Pichia pastoris (BEC10 and SpCas9)

### 1.1 CRISPR/BEC-Ec and FnCpf1-Ec Vector Systems for Genome Editing in E. coli BW25113

The necessary genetic elements for inducible expression of the BEC10 or FnCpf1 nucleases and for the constitutive expression of the guide RNA (gRNA) transcription were provided on three separate vectors (CRISPR/BEC10-Ec, CRISPR/FnCpf1-Ec and CRISPR/gRNA-Ec)

In the following, the construction of the CRISPR/BEC10-Ec vector and the CRISPR/gRNA-Ec systems are described. The CRISPR/FnCpf1-Ec vector systems was constructed in an analogous approach as the CRISPR/BEC10-Ec vector

### Design of the BEC10 coli Protein Expression Vector

The synthetic 3696 bps BEC10 nucleotide sequence was codon optimized for expression in E. coli BW25113, using a bioinformatics application provided by the gene synthesis provider GeneArt (Thermo Fisher Scientific, Regensburg, Germany), SEQ ID NO: 7. For protein expression, the resulting synthetic gene was fused to the inducible araC-ParaBAD inducible promoter system (SEQ ID NO: 8) and the fdT terminator (SEQ ID NO: 9) (Otsuka & Kunisawa, Journal of Theoretical Biology 97 (1982), 415-436) The final BEC10_E. coli protein expression cassette was inserted by Gibson Assembly Cloning (NEB, Frankfurt, Germany) into an E. coli shuttle vector, containing all necessary genetic elements for episomal propagation and selection of recombinant E. coli cells.

### CRISPR/BEC10-Ec Vector System

The complete nucleotide sequence of the constructed CRISPR/BEC10-Ec vector system is provided as SEQ ID NO: 10.

### CRISPR/FnCpf1-Ec Vector System

The complete nucleotide sequence of the constructed CRISPR/fnCpf1-Ec vector system is provided as SEQ ID NO: 11.

### Design of the Guide RNA (gRNA) Expression Vector

The expression of the chimeric gRNA for specific kanamycin gene targeting by BEC10 or FnCpf1 DNA nucleases was driven by the SacB RNA polymerase II promoter from Bacillus megaterium (SEQ ID NO: 12) (Richhardt et al., Applied Microbiology Biotechnology 86 (2010), 1959-1965) and terminated using the transcription T1 and T2 termination region of the E. coli rrnB gene (SEQ ID NO: 13) (Orosz et al., European Journal of Biochemistry 201 (1991), 653-659). The chimeric gRNA was composed of a constant 19 bps BEC family Stem-Loop sequence (SEQ ID NO: 14 (which also works for the FnCpf1 nuclease) fused to the kanamycin target-specific 24 bps spacer sequence (SEQ ID NO: 15) located inside the kanamycin resistance gene of the CRISPR/BEC10-Ec or CRISPR/FnCpf1-Ec vector

The final gRNA expression cassette was inserted by Gibson Assembly Cloning (NEB, Frankfurt, Germany) into an E. coli shuttle vector, containing all necessary genetic elements for episomal propagation and selection of recombinant E. coli cells.

The construction of the final CRISPR/gRNA-Ec vector system was mediated by Gibson Assembly Cloning (NEB, Frankfurt, Germany).

The identity of all cloned DNA elements was confirmed by Sanger-Sequencing at LGC Genomics (Berlin, Germany).

The complete nucleotide sequence of the constructed CRISPR/gRNA-Ec vector system is provided as SEQ ID NO: 16.

### 1.2 E. coli Cultivation and Transformation

### Transformation of Competent E. coli BW25113 Cells

In brief, a single colony of E. coli BW25113 was inoculated in 5 ml LB-Kan medium and incubated for 12 to 14 h at 37° C. on a horizontal shaker at 200 rpm. Overnight grown pre-cultures were diluted into fresh 60 ml LB medium to an optical density at 600 nm (OD600) of 0.06. The inoculated medium was incubated at 30° C. on a horizontal shaker at 200 rpm until the culture reached an optical density at OD600 of 0.2. 600 μl. 20% arabinose was added and the cells were incubated at 30° C. at 200 rpm until the culture reached an optical density at OD600 of 0.5. Cells were transferred into a 50 ml conical tube and harvested by centrifugation at 4° C. for 5 min and 4000×g. Pelleted cells from 50 ml culture were resuspended in 60 ml water and centrifuged at 4° C. for 5 min and 4000×g.

A washing procedure was performed and the cells were resuspended in 30 ml 10% glycerin following a centrifugation at 4° C. for 5 min and 4000×g. In a second washing step, the cells were resuspended in 6 ml 10% glycerin following a centrifugation at 4° C. for 5 min and 4000×g. In the final step, the cells were resuspended in 150 μl 10% glycerin. Aliquots of 25 μl competent cells were stored at −80° C. until use. For transformation procedure, aliquots of competent cells were thawed and 50 ng plasmid DNA was added. Prepared cells were electroporated using 1800 V, 25 μF, 200 Ohm for 5 msec. Subsequently, 975 μL of NEB® 10-beta/Stable Outgrowth Medium was added and 100 μl of the suspension was plated on selective agar plates.

### 1.3 CRISPR/BEC10-Pp Vector Systems for Genome Editing in Pichia pastoris

The necessary genetic elements for constitutive expression of the BEC10 and the guide RNA (gRNA) transcription were provided in an all-in-one CRISPR/BEC10-Pp vector system.

### Design of the BEC10 Protein Expression Cassette

The synthetic 3696 bps BEC10 nucleotide sequence was codon optimized for expression in yeast cells, using a bioinformatics application provided by the gene synthesis provider GeneArt (Thermo Fisher Scientific, Regensburg, Germany), SEQ ID NO: 6. Additionally, the DNA nuclease coding sequence was 5′ extended by a sequence encoding a SV40 nuclear localization signal (NLS) SEQ ID NO: 17 (Kalderon et al., Cell 39 (1984), 499-509). For protein expression, the resulting synthetic 3723 bps gene was fused to the constitutive and bidirectional P. pastoris HTX1 promoter (SEQ ID NO: 18) (Weninger et al., Journal of Biotechnology 235 (2016), 139-149) and the P. pastoris AOX1TT terminator (SEQ ID NO: 19) (Weninger et al., Journal of Biotechnology 235 (2016), 139-149). The final BEC10 protein expression cassette was inserted by Gibson Assembly Cloning (NEB, Frankfurt, Germany) into an E. coli/P. pastoris shuttle vector pPpT6e (EP 3572512 A1), containing all necessary genetic elements for episomal propagation and selection of recombinant E. coli and P. pastoris cells:

For vector propagation and selection of recombinant E. coli cells, the plasmid contained the pUC derived high-copy ColE1 origin of replication and the kanMX6 marker gene (SEQ ID NO: 20) under the control of the bifunctional ILV5/synthetic Em72 promoter (SEQ ID NO: 21), (Weninger et al., Journal of Biotechnology 235 (2016), 139-149), conferring kanamycin resistance to P. pastoris and E. coli respectively. The Pichia autonomously replicating sequence 1 (PARS1) (SEQ ID NO: 22) allowed episomal replication of the pPpT6e shuttle plasmid in P. pastoris cells.

### Design of the Guide RNA (gRNA) Expression Cassette

Ribozyme-based technology was applied to liberate the chimeric gRNA from a RNA polymerase II transcript for specific Ade2 gene targeting by BEC10 DNA nuclease in P. pastoris (Gao & Zhao, Journal of Integrative Plant Biology 56 (2014), 343-349). The bidirectional HTX1 RNA polymerase II promoter was used for expression of 5′ cleaving hammerhead (HH) and 3′ cleaving hepatitis delta virus (HDV) ribozyme flanked gRNA (Weninger et al., Journal of Biotechnology 235 (2016), 139-149).

The chimeric gRNA was composed of a constant 19 bps BEC family Stem-Loop Sequence (SEQ ID NO: 14) fused to the Ade2 target-specific 24 bps spacer sequence (SEQ ID NO: 23). The target spacer sequence was identified in the P. pastoris Ade2 gene (SEQ ID NO: 24) downstream to the nuclease BEC10 specific PAM motive 5′-TTN-3′.

The complete RNA expression cassette composed of the HTX1 RNA polymerase II promoter and the designed HH/HDV ribozyme-flanked chimeric gRNA was provided as a synthetic gene fragment by GeneArt (Thermo Fisher Scientific, Regensburg, Germany).

The construction of the all-in-one CRISPR/BEC10-Pp vector system was completed by cloning the synthetic RNA expression cassette in the prepared E. coli/P. pastoris pPpT6e shuttle vector, containing the BEC10 DNA nuclease expression cassette. The construction of the final CRISPR/BEC10-Pp vector system was mediated by Gibson Assembly Cloning (NEB, Frankfurt, Germany).

The identity of all cloned DNA elements were confirmed by Sanger-Sequencing at LGC Genomics (Berlin, Germany).

### CRISPR/BEC10-Pp All-In-One-Vector System

The complete nucleotide sequence of the constructed CRISPR/BEC10-Pp vector system is provided as SEQ ID NO: 25.

### 1.4 CRISPR/SpCas9-Pp Vector System for Genome Editing in P. pastoris

The necessary genetic elements for constitutive expression of SpCas9 (S. pyogenes Cas9) DNA nuclease and for single guide RNA transcription were provided in an all-in-one CRISPR/SpCas9_Pp vector system.

### Design of the SpCas9 Protein Expression Cassette

Based on the published SpCas9 nucleotide sequence from Streptococcus pyogenes, (Deltcheva et al., Nature 471 (2011), 602-607) DNA synthesis of the yeast codon optimized SpCas9 coding sequence was ordered at GeneArt (Thermo Fisher Scientific, Regensburg, Germany), (SEQ ID NO: 26) for expression in P. pastoris. For nuclear translocation, the SpCas9 DNA nuclease coding sequence was 5′ extended by a sequence encoding a SV40 nuclear localization signal (NLS) (SEQ ID NO: 17). Following the described protein expression strategy for BEC10 DNA nuclease, the resulting synthetic 4134 bps SpCac9 gene was fused to the constitutive and bidirectional P. pastoris HTX1 promotor (SEQ ID NO: 18) and the P. pastoris AOX1TT terminator (SEQ ID NO: 19) (Weninger et al., Journal of Biotechnology 235 (2016), 139-149). The final SpCas9 protein expression cassette was inserted by Gibson Assembly Cloning (NEB, Frankfurt, Germany) into an E. coli/P. pastoris pPpT6e shuttle vector, harboring the identical genetic elements for propagation and selection as already described for the CRISPR/BEC10-Pp vector system.

### Design of the Guide RNA Expression (gRNA) Cassette

Ribozyme-based technology was applied to liberate the chimeric gRNA from from a RNA polymerase II transcript for specific Ade2 gene targeting by SpCas9 DNA nuclease in P. pastoris (Gao & Zhao, Journal of Integrative Plant Biology 56 (2014), 343-349). The bidirectional HTX1 RNA polymerase II promotor was used for expression of 5′ cleaving hammerhead (HH) and 3′ cleaving hepatitis delta virus (HDV) ribozyme flanked gRNA (Weninger et al., Journal of Biotechnology 235 (2016), 139-149).

The chimeric gRNA was composed of the Ade2 target-specific 20 bps spacer sequence (SEQ ID NO: 27) fused to the 80 bps SpCas9 specific sgRNA sequence (SEQ ID NO: 28) The target spacer sequence was identified in the P. pastoris Ade2 gene (SEQ ID NO: 24) downstream to the nuclease SpCa9 specific PAM motive 5′-NGG-3′.

The complete RNA expression cassette composed of the HTX1 RNA polymerase II promoter and the designed HH/HDV ribozyme-flanked chimeric gRNA was provided as a synthetic gene fragment by GeneArt (Thermo Fisher Scientific, Regensburg, Germany).

To generate the final CRISPR/SpCas9-Pp vector system, the synthetic RNA transcription cassette was cloned by Gibson Assembly Cloning (NEB, Frankfurt, Germany) into the prepared E. coli/P. pastoris pPpT6e shuttle vector, containing the SpCas9 DNA nuclease expression cassette. The identity of all cloned DNA elements were confirmed by Sanger-Sequencing at LGC Genomics (Berlin, Germany).

### CRISPR/SpCas9-Pp All-In-One-Vector System

The complete nucleotide sequence of the constructed CRISPR/SpCas9-Pp vector system is provided as SEQ ID NO: 29.

### 1.5 Design of a Homology Directed Repair Template (HDR-Template)

The 1998 bps Ade2 BEC10 and spCas9 HDR-template was designed to generate a site-specific deletion of 1692 bps in the chromosomal P. pastoris Ade2 gene by homologous recombination. Successful homologous recombination resulted in complete Ade2 gene deletion. Within the HDR-template, the introduced Ade2 gene deletion was flanked by 1022 bps and 976 bps sequences homologous to the chromosomal target region. The successful recombination event mediated by the designed HDR-template abolished the already described PAM and protospacer regions in the chromosomal Ade2 gene to prevent the programmed gRNA/BEC10 or gRNA/spCas9 DNA nuclease complex to target the P. pastoris genome again. Furthermore, the introduced gene deletion resulted in Ade2 mutant clones, which were easily recognized by the red color of the colonies, since the mutant cells, deprived of adenine, accumulate red purine precursors in their vacuoles (Ugolini et al., Curr Genet (2006), 485-92).

The complete sequence of the Ade2 HDR-template for BEC10 and SpCas9 is provided as SEQ ID NO: 30.

### 1.6 Pichia pastoris Cultivation and Transformation

### Preparation of Competent P. pastoris CBS7435 Cells

Preparation and transformation of competent P. pastoris CBS7435 cells were performed as described by Wu & Letchworth, Biotechniques (2014), 36, 152-154. In brief, a single colony of P. pastoris was inoculated in 5 ml YPD medium and incubated for 6 to 8 h at 30° C. on a horizontal shaker at 250 rpm. Cells of the preculture were diluted into 100 ml YPD medium to an optical density at 600 nm (OD600 nm) of 0.0025.

The inoculated medium was incubated overnight at 30° C. on a horizontal shaker at 250 rpm until the culture reached an optical density at OD600 nm of 1.0 to 2.0. Cells were harvested by centrifugation for 5 min and 4302×g at room temperature (RT). The cell density of the culture (cells/mL) was estimated from the optical density at OD600 nm according to the following relationship: 1×OD600 nm=5×10⁷ cells/mL. For each transformation, 8×10⁸ cells were suspended in 8 mL of 100 mM LiAc, 10 mM DTT, 0.6 M sorbitol and 10 mM Tris-HCl. The resuspended cells were incubated for 30 min at RT.

The cells were then pelleted by centrifugation for 5 min and 3999×g at RT and resuspended in 1.5 mL of ice-cold 1M sorbitol. The cells were transferred to a 1.5-mL microcentrifuge tube, washed three times with 1.5 mL ice-cold 1 M sorbitol and finally resuspended in 80 μl 1 M ice-cold sorbitol at a final cell density of about 10¹⁰ cells/mL.

### Transformation of Competent P. pastoris Cells

One aliquot of prepared competent cells was mixed with 1 μg of supercoiled plasmid DNA in 5 μL water. For repair of CRISPR/BEC10- or CRISPR/Cas9-induced DNA damages by homologous recombination, 1.5 μg of a linear double-stranded DNA fragment was added to the cells. The total volume of 5 μl DNA solution was not exceeded. The DNA-cell mixture was transferred to a precooled 0.2-cm gap vial and incubated for 5 min on ice. The electroporating pulse was applied at 2 kV, 25 μF, 200Ω using a Bio-Rad Gene Pulser Xcell electroporation system (Bio-Rad Laboratories, Munich, Germany). The electroporated cells were immediately diluted in 1 mL of ice-cold regeneration medium (0.5×YPD, 0.5 M sorbitol) and transferred into a 2 ml microcentrifuge tube. After incubation for 1 h without agitation and 2 h on a horizontal shaker at 250 rpm, the transformed cells were plated on appropriate selective agar plates, depending on the experimental setup.

### Plating of Transformed P. pastoris Cells

For analyzing the efficiencies of various gRNA spacers, in the absence of any homologous repair template, transformed cells were plated on YPD agar plates containing 200 μg/mL geneticin (G418) and incubated at least for 2 days at 30° C.

Ade2 gene disruption and induced red phenotype of mutated P. pastoris cells by BEC10-gRNA- and Cas9-gRNA-mediated integration of co-transformed homologous repair template in the Ade2 target gene was visualized using a simple colony color filter assay: Transformed cells were plated on NC transfer membrane filters (Merck Chemicals, Darmstadt, Germany), applied directly to the surface of YPD agar plates supplemented with 200 μg/mL geneticin (G418) and 50 μg/mL adenine. After incubation for at least 2 days at 30° C., the filters with grown cells were transferred onto minimal media agar plates supplemented with 200 μg/mL geneticin (G418) and 5 μg/mL adenine

### EXAMPLE 2

### Vector Targeting in E. coli to Demonstrate the Novel DNA Targeting Mechanism of BEC Family Nucleases in Comparison to FnCpf1

To demonstrate the novel DNA targeting mechanism of BEC family nucleases experiments were carried out using two different vectors co-transformed into E. coli. In two different approaches, the CRISPR/BEC10-Ec vector or the CRISPR/FnCpf1-Ec vector was co-transformed together with the CRISPR/gRNA-Ec vector including a spacer sequence targeting the Ampicillin resistance gene located on the CRISPR/BEC10-Ec vector or the CRISPR/FnCpf1-Ec vector (Schematic drawing of the experimental setup is shown in FIG. 1). Using a nuclease with a classical DNA targeting activity (double strand break) the activation of the CRISPR nuclease would linearize the CRISPR/BEC10-Ec or the CRISPR/FnCpf1-Ec vector leading to a disruption of the open reading frame of the Ampicillin resistance gene and prevent the propagation of the vector. Therefore, the cells would become sensitive for Ampicillin, due to deletion of the Ampicillin resistance gene located on the CRISPR/BEC10-Ec or the CRISPR/FnCpf1-Ec vector, but remain resistant against Kanamycin, since the Kanamycin resistance gene located on the CRISPR/gRNA-Ec vector remains intact.

To directly compare the DNA targeting mechanism of a BEC family (BEC10) and a classical (FnCpf1) nuclease the following experiments were carried out:
1. The CRISPR/BEC10-Ec vector and the CRISPR/gRNA-Ec vector were co-transformed into E. coli cells and grown/selected on plates containing Ampicillin & Kanamycin and plates containing only Kanamycin.
2. The CRISPR/FnCpf1-Ec vector and the CRISPR/gRNA-Ec vector were co-transformed into E. coli cells and grown/selected on plates containing Ampicillin & Kanamycin and plates containing only Kanamycin.
3. Negative control (NC) experiments for setup 1 and 2 were carried out using the same experimental approaches but with a nonsense spacer sequence (does not match a sequence in one of the vectors or the E. coli genome) in the CRISPR/gRNA-Ec vector.

All plates were visually evaluated by counting the number of grown colonies.

### Results

All experiments were carried out in 5 biological replicates and the results obtained from these replicates were combined to evaluate the DNA targeting mechanism of BEC10 in comparison to FnCpf1 (Exemplary plates are shown in FIG. 2).

### FnCpf1

The results obtained using the FnCpf1 nuclease showed the expected outcome. The plates containing the Kanamycin antibiotic showed a comparable number of grown E. coli colonies to the negative control (NC) because the cells were still resistant to Kanamycin as the resistance gene is located on the CRISPR/gRNA-Ec vector that is not targeted by the gRNA. In contrast to this, the plates containing Ampicillin and Kanamycin showed a strong colony reduction (>99%) in contrast to the negative control because the vector containing the Ampicillin resistance gene (CRISPR/FnCpf1-Ec) was targeted and depleted by the FnCpf1 nuclease.

### BEC10

Surprisingly, the results obtained using the BEC10 nuclease showed a different growth pattern. The plates containing the Kanamycin antibiotic and the plates containing the Ampicillin and Kanamycin antibiotic showed a strong colony reduction (>99%) in comparison to the negative control. As described for the FnCpf1 experiments, the colony reduction was expected on plates containing Ampicillin and Kanamycin (targeting/depletion of the Ampicillin containing vector). In addition to this, the plates containing only Kanamycin showed no colony reduction using the FnCpf1 nuclease but a strong colony reduction using the BEC10 nuclease.

### Conclusion

The explanation for these significant differences between the BEC10 and FnCpf1 results is the novel mode of action of the BEC family nucleases: The initial sequence specific binding of the spacer sequence (incorporated into the gRNA) to the protospacer region (target region) on the vector activates the BEC nuclease and triggers an unspecific targeting of double stranded DNA. Therefore, the DNA targeting activity of the BEC nucleases (once specifically activated) degrades dsDNA and is able to “jump” to other DNA strands inside of the same cell. This is a type of reaction that can be described as collateral activity, since even DNA without a target region is degraded once the nuclease is activated.

In the shown experiment, the BEC nuclease was activated by the protospacer sequence located on the CRISPR/BEC10-Ec vector and once activated the activity “jumped” over and targeted the CRISPR/gRNA-Ec and/or the E. coli genome leading to the strong colony reduction on plates containing Kanamycin only.

To our knowledge, a nuclease that is specifically activated by a spacer/protospacer pairing and once activated has collateral dsDNA cutting activity has never been described before. This novel activity pattern can be used in various applications where classical CRISPR nucleases are non-functional or have a limited applicability (e.g. Example 3 and Example 5).

### EXAMPLE 3

### Precise Genome Editing in P. pastoris Using the BEC10 Nuclease in Comparison to SpCas9

To demonstrate one of the advantages of the novel mode of action of BEC family nucleases, genome editing experiments were carried out in an organism that is natively capable to perform non-homologous end joining (NHEJ) DNA repair.

### Experimental Setup

In this example, the CRISPR/BEC10-Pp or CRISPR/SpCas9-Pp vector system and HDR template were used to knock out the Ade2 gene in P. pastoris.

Ade2 is a non-essential gene of P. pastoris but a knockout leads to a red phenotype of the colonies, since the mutant cells accumulate red purine precursors in their vacuoles (Ugolini et al., Curr Genet (2006), 485-92). Due to this easy readout, the knockout of the Ade2 gene can be utilized as a screening system to monitor the ability of CRISPR Cas proteins to function as a genome-editing tool.

In this approach, the knockout of the Ade2 gene was used to monitor the genome editing activity of the BEC10 nuclease in comparison to SpCas9 in two experimental setups.
1. Ade2 knockout via NHEJ (without using a HDR template)
2. Specific Ade2 knockout using homology directed repair (introduction of a HDR template leading to a site-specific deletion of the Ade2 gene also eliminating the PAM and protospacer sequence)

In summary, the CRISPR/BEC10-Pp or CRISPR/SpCas9-Pp expression constructs with or without a homology directed repair template were transformed into P. pastoris cells and plated as described in Example 1.6.

In parallel, negative control experiments using the CRISPR/BEC10-Pp or CRISPR/SpCas9-Pp expression constructs lacking a spacer sequence targeting the Ade2 gene were performed to demonstrate the dependency of the Cas proteins to be guided to the target DNA region by a specific spacer.

After transformation and 48 h incubation at 30° C. the culture plates were analyzed by counting the number of grown colonies and by the evaluation of their phenotype (red or white).

### Results

All experiments were carried out in 5 biological replicates and the results obtained from these replicates were combined to evaluate the genome editing activity of BEC10 in comparison to SpCas9 (Exemplary plates are shown in FIG. 3).

In the first experimental setup the CRISPR/BEC10-Pp or CRISPR/SpCas9-Pp expression construct was transformed into P. pastoris cells without using a HDR template (−HDR). In comparison to the negative control experiments the active SpCas9 and BEC10 nuclease lead to a strong colony reduction (>99%) with an even stronger decrease of the colonies when using the BEC10 nuclease. Furthermore, in the experimental approach using the Cas9 nuclease≈40% of the remaining cells showed a red (Ade2 knockout) phenotype in contrast to the cells treated with the BEC10 nuclease where 0% of the cells showed a red phenotype.

In the second experimental setup the CRISPR/BEC10-Pp or CRISPR/SpCas9-Pp expression construct was transformed into P. pastoris cells together with the HDR template (+HDR). In comparison to the negative control experiments the active SpCas9 and BEC10 nucleases lead to a strong colony reduction (>98%) which was a slightly less dramatic decrease in the overall cell numbers compared to the experiments without the HDR template. Furthermore, the majority of surviving cells showed an Ade2 knockout phenotype for SpCas9 and BEC10 (SpCas9≈73%/BEC10≈90%). Additionally, 40 colonies (20 SpCas9 treated and 20 BEC10 treated) showing an Ade2 knockout phenotype were further analyzed by Sanger sequencing showing that 14 out of the 20 colonies treated with Cas9 had incorporated the HDR template into their genome and 6 were edited by NHEJ. In contrast to this, 20 out of 20 colonies analyzed after the BEC10 treatment showed the introduction of the HDR template into the Ade2 gene into their genome.

### Conclusion

Even though the SpCas9 and BEC nucleases both lead to a significant overall colony reduction after activation, the Ade2 gene editing showed unequivocally different results between both nucleases.

As P. pastoris is an organism that is intrinsically able to repair DNA double strand breaks using NHEJ, the targeting of the Ade2 gene using SpCas9 leads to Ade2 knockout colonies due to frameshift mutations caused by the faulty NHEJ mechanism. In contrast to this, the cells treated with the BEC10 nuclease did not show Ade2 knockout colonies, which can be explained by the novel mode of action of the BEC family nucleases. BEC family nucleases exhibit a completely different mode of action which prevents NHEJ because the activation of the BEC nuclease induces a collateral dsDNA activity leading to cell death without giving the cell the opportunity to prevent death by NHEJ derived DNA repair.

In contrast to this, Ade2 knockout colonies were present for SpCas9 and BEC10 when using a HDR template to knockout the gene. For SpCas9 the introduced dsDNA break inside the Ade2 gene forces the cell to repair this break. To do so, the cell has two options: A: introduce the HDR template or B: repair of the DNA break using NHEJ. As P. pastoris prefers homology directed repair over NHEJ≈70% of the edited cells introduced the HDR template into their genome and ≈30% of the edited cells performed NHEJ mediated DNA repair.

In contrast to this, the BEC10 nuclease prevents the cells from NHEJ mediated DNA repair by killing the cells, once the BEC nuclease is activated. Therefore, the BEC10 nuclease forces the cells to introduce the HDR template into their genome before the BEC nuclease is activated. As the PAM and protospacer sequence within the genome are deleted by the integration of the HDR template, the protospacer sequence matching the spacer sequence of the BEC gRNA is no longer present in the genome and the BEC nuclease stays inactive. Those cells, in which the HDR template is integrated into the genome and the BEC nuclease remains inactive are able to survive the treatment explaining why 100% of the BEC edited cells incorporated the HDR template into their genome.

### Outlook

Most of the higher organisms (eukaryotes) are able to perform NHEJ. Using genome editing tools, this mechanism can be used to knock out a gene of interest in a rather unspecific way (introduction of indels by the faulty NHEJ repair mechanism). However, as soon as it comes to precise knock-outs or knock-ins using a HDR template, the NHEJ mechanism leads to a lot of technical problems, as part of the edited cells will use the NHEJ pathway to repair their DNA instead of introducing the HDR template thus leading to mixed cell populations with unwanted edits. Using BEC family nucleases can overcome this limitation, as BEC nucleases show a novel mode of action to target dsDNA inhibiting the cells' ability to perform NHEJ to survive a DNA double strand break. Furthermore, the cells are forced to introduce the HDR template into their genome to prevent cell death leading to a very efficient way to perform precise gene knock-outs or knock-ins using BEC nucleases

### EXAMPLE 4

### Expression and Purification of BEC10 RNPs for HEK Cell Transfection

### 4.1 CRISPR/BEC-FLAG-Ec and CRISPR/gRNA-Ec Vector Systems for BEC Expression (E. coli) and RNP Purification

The necessary genetic elements for inducible expression and FLAG tag purification of the BEC10 nuclease and for the constitutive expression of the guide RNA (gRNA) transcription were provided on three separate vectors (CRISPR/BEC10-FLAG-Ec, CRISPR/gRNA-EGFP-Ec and CRISPR/gRNA-NC-Ec).

### Design of the CRISPR/BEC-FLAG-Ec Protein Expression Vector

The synthetic 3696 bps BEC10 nucleotide sequence was codon optimized for expression in E. coli BW25113, using a bioinformatics application provided by the gene synthesis provider GeneArt (Thermo Fisher Scientific, Regensburg, Germany). For protein expression, the resulting synthetic gene was fused to the inducible araC-ParaBAD inducible promoter system and the fdT terminator (Otsuka & Kunisawa, Journal of Theoretical Biology 97 (1982), 415-436). Additionally, the DNA nuclease coding sequence was 3′ extended by a sequence encoding a nucleoplasmin nuclear localization signal (NLS) and two SV40 NLS. At the 5′ end of the DNA nuclease coding sequence, the sequence was extended by a sequence encoding a myc NLS. For purification of the DNA nuclease a FLAG tag was linked by a linker to the myc NLS to the 5′ coding sequence of the DNA nuclease.

The final BEC10_E. coli protein expression cassette was inserted by Gibson Assembly Cloning (NEB, Frankfurt, Germany) into an E. coli shuttle vector, containing all necessary genetic elements for episomal propagation and selection of recombinant E. coli cells.

### CRISPR/BEC10-FLAG-Ec Vector System

The complete nucleotide sequence of the constructed CRISPR/BEC10-FLAG-Ec vector system is provided as SEQ ID NO: 31.

### Design of the Guide RNA (gRNA) Expression Vector for EGFP Targeting (CRISPR/gRNA-EGFP-Ec) and the Negative Control (CRISPR/gRNA-NC-Ec)

The design of the gRNA expression vector is described in Example 1.1. In contrast to the vector described in 1.1 the spacer sequence targeting the kanamycin gene is exchanged for a spacer sequence targeting the EGFP gene (SEQ ID NO: 32) or a negative control (NC) sequence (SEQ ID NO: 33) that does not match with any target area in the used HEK cells.

The complete nucleotide sequence of the constructed CRISPR/gRNA-EGFP-Ec vector system is provided as SEQ ID NO: 34.

The complete nucleotide sequence of the constructed CRISPR/gRNA-NC-Ec vector system is provided as SEQ ID NO: 35.

### Nuclease Expression

In brief, a single colony of E. coli BW25113+CRISPR/BEC10-FLAG-Ec+CRISPR/gRNA-EGFP-Ec for BEC10-EGFP RNP expression or E. coli BW25113+CRISPR/BEC10-FLAG-Ec+CRISPR/gRNA-NC-Ec for BEC10-NC RNP expression was inoculated in 5 ml LB-Kan-Amp medium and incubated for 12 to 14 h at 37° C. on a horizontal shaker at 200 rpm. The overnight grown pre-cultures were diluted into fresh 60 ml LB medium to an optical density at 600 nm (OD600) of 0.05. The inoculated medium was incubated at 30° C. on a horizontal shaker at 200 rpm until the culture reached an optical density at OD600 of 0.2. The incubation temperature was lowered to 21° C. At an optical density OD600 of 0.5 600 μl 20% L-arabinose was added and the cells were incubated at 21° C. at 200 rpm for approximately 21 hours until the culture reached an optical density at OD600 of 4. The culture was transferred into a 50 ml conical tube and cells were harvested by centrifugation at 4° C. for 10 min and 4000×g. Pelleted cells from 90 ODV culture were stored at −20° C.

### Cell Disruption and FLAG Tag Purification

90 ODV pelleted cells were resuspended in 3 ml TBS (50 mM TRIS-HCl, 150 mM NaCl, pH 7.4) and disrupted by ultrasonic cell disruption using Branson Sonifier 250 with four ultrasonic cycles with 50% duty cycle, 2.5 output for 30 sec. Between each cycle the cells were cooled down for 1 min on ice. The soluble fraction was separated from the insoluble fraction by centrifugation at 4° C. for 10 min and 4500 rpm.

The nuclease and gRNA (already coupled to each other) were purified by Pierce™ magnetic anti-DYKDDDDK-agarose (ThermoFisher Scientific, Regensburg Germany). 120 μl magnetic bead aliquots were used for purification of 1.5 ml soluble cell fraction. The magnetic beads were equilibrated two times with 1 ml TBS buffer (50 mM Tris-HCl pH 7.5, 150 mM NaCl). The supernatant was removed using a magnetic stand. 1.5 ml soluble fraction was incubated with the magnetic beads for 18-20 hours on a rotator at 4° C. After binding, the magnetic beads were washed four times with 1 ml TBS buffer (50 mM Tris-HCl pH 7.5, 150 mM NaCl) and once with 1 ml of LC-MS analyzed water (TH Geyer, Renningen, Germany) and the supernatant was removed in a magnetic stand. The nuclease/gRNA complex was eluted from the beads by the addition of 50 μl 1.5 mg/ml Pierce™ 3×DYKDDDK-peptide (ThermoFisher Scientific, Regensburg, Germany) solved in dPBS (PAN Biotech, Aidenbach, Germany) and incubated for 30 min at room temperature on a rotator. The supernatants containing the purified BEC10-EGFP or BEC10-NC ribonucleoproteins (RNPs) were removed from the beads in the magnetic stand and stored at 4° C.

### HEK-EGFP and HEK-DsRed Cell Generation

To generate the two types of HEK cells the commercially available Flp-In™ T-REx™ 293 Cell Line (Thermo Fisher) was used in combination with the Flp-In™ T-REx™ Core Kit (Thermo Fisher). To integrate the gene of interest a DNA fragment containing the EGFP (enhanced Green Fluorescent Protein) (SEQ ID NO: 36) or DsRed (Red Fluorescent Protein) (SEQ ID NO: 37) coding sequence was locus specifically integrated into the genome of the Flp-In™ T-REx™ 293 Cells following the instructions for use provided with the Flp-In™ T-REx™ Core Kit leading to two identical cell lines solely different by the inserted genes (HEK-EGFP and HEK-DsRed).

### HEK Cell Transfection and Cultivation

Cells were seeded and cultivated in medium (DMEM, 8% FCS tetracycline free, 4 mM L-Glutamine; PAN Biotech, Aidenbach, Germany, #P04-03600, #P30-3602, #P04-80100)+1 μg/ml tetracycline (Merck KGaA, Darmstadt, Germany, #T7660) (to induce the expression of the EGFP and DsRed genes) to reach 70% to 90% confluency on the day of transfection. After rinsing with 5 ml of 1×DPBS (without Ca²⁺ and Mg²; PAN Biotech, Aidenbach, Germany, #P04-53500), cells were detached by adding 1 ml pre-warmed TrypLE (Thermo Fisher Scientific, Regensburg, Germany) to the 10 cm petri dish and incubated at 37° C. for 2 minutes. After adding 5 ml of pre-warmed medium the cell number of the suspension was determined with a CASY cell counter (OLS OMNI Life Science GmbH & Co KG, Bremen, Germany). 1×10⁵ cells were transferred in a 5 ml conical tube and centrifuged at room temperature for 5 minutes and 200 g. The supernatant was removed and the pelleted cells were resuspended in 1 ml 1×DPBS (without Ca²⁺ and Mg²⁺) and centrifuged at room temperature for 5 minutes and 200 g. For electroporation with the Neon™ Transfection System (Thermo Fisher Scientific, Regensburg, Germany) applying the Neon™ 10 μl Kit (Thermo Fisher Scientific, Regensburg, Germany) the supernatant was removed again and the cells were resuspended in 10 μl Resuspension Buffer R containing 50 pmol of purified BEC10-EGFP or BEC10-NC RNPs. The cell-RNP suspension was then aspirated with a 10 μl Neon™ transfection tip and transferred to the Neon™ transfection tube containing 3 ml of Electrolytic Buffer E. Cells were then electroporated with a single pulse of 1600 V and 20 ms pulse width. Transfected cells were cultivated in 24 wells with 500 μl pre-warmed (37° C.) medium containing 1× Gibco™ antibiotic/antimycotic solution (Thermo Fisher Scientific, Regensburg, Germany) and 15 μg/ml Gibco™ gentamycin (Thermo Fisher Scientific, Regensburg, Germany) at 37° C. and 5% CO₂. After 24 h of cultivation 1 μg/ml tetracycline was added to the medium.

After reaching a confluency of approximately 70% to 90% the transfected cells in 24 wells were detached, using pre-warmed (37° C.) TrypLE and transferred to 10 cm petri dishes and cultivated for another 5 days in medium+1 μg/ml tetracycline (medium was exchanged after 3 days of cultivation) before visual evaluation by bright field and fluorescence (EGFP=Ex λ488 nm, Em λ507 nm/DsRed=Ex λ558 nm, Em λ583) microscopy (Leica DM II LED—Leica Microsystems GmbH, Wetzlar, Germany) and flow cytometrical cell counting (CyFlow Space—Sysmex Deutschland GmbH, Norderstedt, Germany).

### FACS (Fluorescence-Activated Cell Sorting) Analysis

Cells were rinsed with 5 ml 1×DPBS and detached with 1 ml of TrypLE at 37° C. for 10 minutes. To stop the detachment process, 9 ml of DMEM were added and the cell number was determined by CASY cell counter. After transferring the cell suspension to 50 ml conical tubes, cells were pelleted by centrifugation for 5 minutes at room temperature at 200 g and then resuspended in 1×DPBS in an appropriate volume to adjust the cell number to 1×10⁶ cells/ml. The cell suspension was then visually analyzed by FACS (CyFlow Space—Sysmex Deutschland GmbH, Norderstedt, Germany) to evaluate the EGFP staining of the cells following the instructions for use provided by the manufacturer.

### EXAMPLE 5

### Target Specific Cell Depletion of Mammalian Cells

To demonstrate the usage of BEC family nucleases in an application area where classical CRISPR nucleases cannot be applied, experiments were carried out showing the BEC10 induced depletion of mammalian cells based on a specific target area.

### Experimental Setup

To demonstrate the specific depletion of cells using the novel mode of action of the BEC family nucleases (collateral activity after an initial, guide specific activation), experiments were carried out using a 50/50 mixture of two types of HEK-cells (HEK-EGFP and HEK-DsRed). Both types of HEK cells are almost identical Flp-In™ T-REX™ 293 cells except from the locus specific integration of the EGFP or DsRed gene. A 50/50 mixture of both cell types was cultivated like described in Example 4 and the BEC10 nuclease in combination with a spacer sequence that specifically binds to a region in the EGFP gene (and does not match to the rest of the genome or the DsRed gene) was transfected into the cells like described in Example 4. After cultivating the cell mixture for seven days the cells were visually evaluated using microscopy (bright field and fluorescence) and counted via FACS to demonstrate the specific cell depletion of the EGFP carrying cells without affecting the viability of the DsRed carrying cells.

In parallel, negative control experiments using the BEC10 nuclease in combination with a spacer sequence that does not bind to any sequence in the HEK-EGFP or HEK-DsRed cells were performed to demonstrate the dependency of the guide specific BEC10 activation for the targeted cell depletion.

### Results

All experiments were carried out in six biological replicates and the results obtained from these replicates were combined to evaluate the cell depletion efficiency of the BEC10 nuclease. Exemplary microscopy images and FACS results are shown in FIG. 4.

After seven days of cultivation the 50/50 mixtures of HEK-EGFP and HEK-DsRed cells were evaluated by microscopy (bright field and fluorescence) and FACS counting.

The negative control experiments using the BEC10-NC RNPs (BEC10 nuclease in combination with a spacer sequence that does not match with a sequence from the HEK-EGFP or HEK-DsRed cells) showed normal cell growth on the plates (FIG. 4A top image) (around half of the grown cells showed an EGFP staining (FIG. 4A middle image) and the other half showed a DsRed staining (FIG. 4A bottom image). In support of this visual evaluation, the FACS counting of the grown colonies of the negative control (FIG. 4C upper diagram) showed that ≈55% of the counted cells were identified as EGFP negative (peak on the left side of the diagram) and ≈45% as EGFP positive (peak on the right side of the diagram) cells confirming that the 50/50 mixture of both cell types was not significantly changed throughout the seven days of cultivation in this negative control setup.

The BEC specific cell depletion experiments using the BEC10-EGFP RNPs (BEC10 nuclease in combination with a spacer sequence that specifically matches a sequence from the EGFP gene) showed a significant reduction of grown cells after seven days compared to the negative control (FIG. 4B upper image vs. FIG. 4A upper image) indicating a depletion of cells during the seven days cell growth period. Furthermore, the fluorescence images revealed that only two cells in the field of view showed an EGFP staining (FIG. 4B middle image) indicating the strong depletion of the HEK-EGFP positive cells whereas the viability of HEK-DsRed cells (FIG. 4B bottom image) stayed unaffected by the BEC10 treatment (almost all of the grown cells showed a DsRed staining). The FACS counting also supported these results as the right peak (showing the EGFP positive cells) was almost completely vanished whereas the left peak showed a strong signal for EGFP negative cells. Further evaluation of the FACS results showed that less than 1.5% of the overall cell population was EGFP positive and more than 98.5% was EGFP negative confirming the specific cell depletion of the EGFP positive cells.

### Conclusion

The Example demonstrates the usage of the novel and unique mode of action of BEC family nucleases for target specific depletion of mammalian cells.

Non homologues end joining (NHEJ) is the preferred DNA repair mechanism in most of the higher eukaryotic cells (e.g. mammalian and plant cells) meaning that DNA double strand breaks introduced by classical CRISPR nucleases will be repaired by the NHEJ mechanism (often leading to short Indels (insertion or deletion)) allowing the cell to survive the DNA damage.

In contrast to this, BEC family nucleases induce collateral dsDNA degradation after an initial, guide specific activation overcharging the repair machinery of the target cell leading to cell death. Due to the PAM and spacer sequence specific initial activation of the BEC family nucleases this novel mode of action can be used for the selective depletion of all cells carrying the marker sequence without affecting the viability of the surrounding cells that do not carry this specific sequence.

In the future, this novel and unique mode of action can be used in various application areas, e.g. the target specific depletion of prokaryotic and eukaryotic cells, including e.g. cancer, autoreactive immune or virus-infected cells, and the enrichment of classical genome editing events by the subsequent depletion of all non-edited cells.

1. A method for the genome editing of cells at a target locus by homology directed repair (HDR) within a cell population and concurrently enriching within said cell population the HDR-genome edited cells, wherein the method comprises
(A) introducing into the cells within the cell population one or more nucleic acid molecules, said one or more nucleic acid molecules encoding in expressible form
(i) a CRISPR nuclease comprising or consisting of
(a) an amino acid sequence of any one of SEQ ID NO: 1, 2 or 3;
(b) an amino acid sequence being encoded by the nucleotide sequence of SEQ ID NO: 4, 5, 6 or 7;
(c) an amino acid sequence being at least 80% identical to the amino acid sequence of (a), or
(d) an amino acid sequence being encoded by a nucleic acid sequence being at least 80% identical to the nucleotide sequence of (b),
(ii) a guide RNA comprising (a) a first segment comprising a nucleotide sequence that is complementary to a sequence at the target locus and (b) a second segment that interacts with the CRISPR nuclease of (i), and
a nucleic acid molecule comprising, consisting of, or encoding
(iii) a donor template with homology to the target locus; or
(A′) introducing into the cells within the cell population
(i) a CRISPR nuclease comprising or consisting of
(a) an amino acid sequence of any one of SEQ ID NO: 1, 2 or 3;
(b) an amino acid sequence being encoded by the nucleotide sequence of SEQ ID NO: 4, 5, 6 or 7;
(c) an amino acid sequence being at least 80% identical to the amino acid sequence of (a), or
(d) an amino acid sequence being encoded by a nucleic acid sequence being at least 80% identical to the nucleotide sequence of (b),
a nucleic acid molecule, said nucleic acid molecule encoding in expressible form
(ii) a guide RNA comprising (a) a first segment comprising a nucleotide sequence that is complementary to a sequence at the target locus and (b) a second segment that interacts with the CRISPR nuclease of (i), and
a nucleic acid molecule comprising, consisting of, or encoding
(iii) a donor template with homology to the target locus; or
(A″) introducing into the cells within the cell population
(i) a ribonucleoprotein complex (RNP) comprising or consisting of
a CRISPR nuclease comprising or consisting of
(a) an amino acid sequence of any one of SEQ ID NO: 1, 2 or 3;
(b) an amino acid sequence being encoded by the nucleotide sequence of SEQ ID NO: 4, 5, 6 or 7;
(c) an amino acid sequence being at least 80% identical to the amino acid sequence of (a), or
(d) an amino acid sequence being encoded by a nucleic acid sequence being at least 80% identical to the nucleotide sequence of (b),
in complex with a guide RNA comprising (a) a first segment comprising a nucleotide sequence that is complementary to a sequence at the target locus and (b) a second segment that interacts with the CRISPR nuclease; and
a nucleic acid molecule comprising, consisting of, or encoding
(ii) a nucleic acid molecule encoding in expressible form a donor template with homology to the target locus; and
(B) culturing the cells under conditions wherein the target locus with the cells of the cell population becomes genome edited by homology directed repair (HDR) and the CRISPR nuclease concurrently enriches within said cell population the HDR-genome edited cells.

2. The method of claim 1, wherein the method further comprises
(C) isolating one or more cells, wherein the target locus has been genome edited by homology directed repair (HDR).

3. The method of claim 1, wherein the donor template introduces one or more mutations at the target locus.

4. The method of claim 1, wherein the donor template inserts one or more nucleotides at the target locus.

5. The method of claim 1, wherein the donor template deletes one or more nucleotides at the target locus.

6. The method of claim 1, wherein the donor template substitutes one or more nucleotides at the target locus.

7. The method of claim 1, wherein the donor template comprises or consists of a nucleotide sequence with one or more intended mutations flanked by nucleotide sequences being homologous to the target locus.

8. A method for the selective depletion of cells comprising a target locus within a cell population, wherein the method comprises
(A) introducing into the cells within the cell population one or more nucleic acid molecules, said one or more nucleic acid molecules encoding in expressible form
(i) a CRISPR nuclease comprising or consisting of
(a) an amino acid sequence of any one of SEQ ID NO: 1, 2 or 3;
(b) an amino acid sequence being encoded by the nucleotide sequence of SEQ ID NO: 4, 5, 6 or 7;
(c) an amino acid sequence being at least 80% identical to the amino acid sequence of (a), or
(d) an amino acid sequence being encoded by a nucleic acid sequence being at least 80% identical to the nucleotide sequence of (b), and
(ii) a guide RNA comprising (a) a first segment comprising a nucleotide sequence that is complementary to a sequence at the target locus and (b) a second segment that interacts with the CRISPR nuclease of (i); or
(A′) introducing into the cells within the cell population
(i) a CRISPR nuclease comprising or consisting of
(a) an amino acid sequence of any one of SEQ ID NO: 1, 2 or 3;
(b) an amino acid sequence being encoded by the nucleotide sequence of SEQ ID NO: 4, 5, 6 or 7;
(c) an amino acid sequence being at least 80% identical to the amino acid sequence of (a), or
(d) an amino acid sequence being encoded by a nucleic acid sequence being at least 80% identical to the nucleotide sequence of (b),
and one or more nucleic acid molecules, said one or more nucleic acid molecules encoding in expressible form
(ii) a guide RNA comprising (a) a first segment comprising a nucleotide sequence that is complementary to a sequence at the target locus and (b) a second segment that interacts with the CRISPR nuclease of (i); or
(A″) introducing into the cells within the cell population
(i) a ribonucleoprotein complex (RNP)
comprising or consisting of
(a) an amino acid sequence of any one of SEQ ID NO: 1, 2 or 3;
(b) an amino acid sequence being encoded by the nucleotide sequence of SEQ ID NO: 4, 5, 6 or 7;
(c) an amino acid sequence being at least 80% identical to the amino acid sequence of (a), or
(d) an amino acid sequence being encoded by a nucleic acid sequence being at least 80% identical to the nucleotide sequence of (b),
in complex with a guide RNA comprising (a) a first segment comprising a nucleotide sequence that is complementary to a sequence at the target locus and (b) a second segment that interacts with the CRISPR nuclease; and
(B) culturing the cells under conditions wherein the CRISPR nuclease selectively depletes the cells within the cell population that comprise the target locus.

9. The method of claim 1—wherein the cells within the population are capable of repairing double-stranded DNA breaks through non-homologous end joining (NHEJ).

10. The method of claim 1, wherein the cells within the population are prokaryotic cells or eukaryotic cells, preferably vertebrate cell, and most preferably mammalian cells.

11. The method of claim 1, wherein the method is an in vitro or ex vivo method.

12. (A) One or more nucleic acid molecules, said one or more nucleic acid molecules encoding in expressible form
(i) a CRISPR nuclease comprising or consisting of
(a) an amino acid sequence of any one of SEQ ID NO: 1, 2 or 3;
(b) an amino acid sequence being encoded by the nucleotide sequence of SEQ ID NO: 4, 5, 6 or 7;
(c) an amino acid sequence being at least 80% identical to the amino acid sequence of (a), or
(d) an amino acid sequence being encoded by a nucleic acid sequence being at least 80% identical to the nucleotide sequence of (b), and
(ii) a guide RNA comprising (a) a first segment comprising a nucleotide sequence that is complementary to a sequence at the target locus; and (a) a second segment that interacts with the CRISPR nuclease of (i); or
(A′) (i) a CRISPR nuclease comprising or consisting of
(a) an amino acid sequence of any one of SEQ ID NO: 1, 2 or 3;
(b) an amino acid sequence being encoded by the nucleotide sequence of SEQ ID NO: 4, 5, 6 or 7;
(c) an amino acid sequence being at least 80% identical to the amino acid sequence of (a), or
(d) an amino acid sequence being encoded by a nucleic acid sequence being at least 80% identical to the nucleotide sequence of (b),
and one or more nucleic acid molecules, said one or more nucleic acid molecules encoding in expressible form
(ii) a guide RNA comprising (a) a first segment comprising a nucleotide sequence that is complementary to a sequence at the target locus; and (a) a second segment that interacts with the CRISPR nuclease of (i); or
(A″) a ribonucleoprotein complex (RNP) comprising or consisting of
(a) an amino acid sequence of any one of SEQ ID NO: 1, 2 or 3;
(b) an amino acid sequence being encoded by the nucleotide sequence of SEQ ID NO: 4, 5, 6 or 7;
(c) an amino acid sequence being at least 80% identical to the amino acid sequence of (a), or
(d) an amino acid sequence being encoded by a nucleic acid sequence being at least 80% identical to the nucleotide sequence of (b),
in complex with a guide RNA comprising (a) a first segment comprising a nucleotide sequence that is complementary to a sequence at the target locus; and (a) a second segment that interacts with the CRISPR nuclease
for use in treating a disease by the selectively depletion of cells comprising a target locus being associated with the disease to be treated.

13. A vector comprising in expressible form
(i) a CRISPR nuclease comprising or consisting of
(a) an amino acid sequence of any one of SEQ ID NO: 1, 2 or 3;
(b) an amino acid sequence being encoded by the nucleotide sequence of SEQ ID NO: 4, 5, 6 or 7;
(c) an amino acid sequence being at least 80% identical to the amino acid sequence of (a), or
(d) an amino acid sequence being encoded by a nucleic acid sequence being at least 80% identical to the nucleotide sequence of (b),
(ii) a guide RNA comprising (a) a first segment comprising a nucleotide sequence that is complementary to a sequence at the target locus; and (a) a second segment that interacts with the CRISPR nuclease of (i), and
(iii) optionally a donor template with homology to the target locus.

14. A method for genome editing of cells at a target locus by homology directed repair (HDR) within a cell population and concurrently enriching within said cell population the HDR-genome edited cells, and/or for the selective depletion of cells comprising a target locus within a cell population comprising using a CRISPR nuclease comprising
(a) an amino acid sequence of any one of SEQ ID NO: 1, 2 or 3;
(b) an amino acid sequence being encoded by the nucleotide sequence of SEQ ID NO: 4, 5, 6 or 7;
(c) an amino acid sequence being at least 80% identical to the amino acid sequence of (a), or
(d) an amino acid sequence being encoded by a nucleic acid sequence being at least 80% identical to the nucleotide sequence of (b).

15. The method of claim 1, wherein the one or more nucleic acid molecules, or the CRISPR nuclease and the guide RNA, or the RNP is administered to a patient to treat a disease by selective depletion of cells comprising a target locus associated with the disease to be treated, wherein the sequence identity of at least 80% is at least 85%, at least 90% or at least 95%

16. The vector of claim 13, wherein the sequence identity of at least 80% is at least 85%, at least 90% or at least 95%.

17. A method for genome editing of cells at a target locus by homology directed repair (HDR) within a cell population and concurrently enriching within said cell population the HDR-genome edited cells, and/or for the selective depletion of cells comprising a target locus within a cell population according to claim 14, wherein the amino acid sequence identity of at least 80% is at least 85%, at least 90% or at least 95%.


SEQUENCE LISTING (37 sequences)

SEQ ID NO: 1 - protein, 1231 aa, synthetic construct
  REGION 1..1231: note = BEC85 AA Sequence
MKSFENSCQISKTLRFGATLKEDKKKCKSHLELQEFVDISYKTLKSSATIAESLNEIELVKKCERCYSEIIKFHEAWGQIYCRTNQIAVYKDWYRQLARKARFDVGDQNSQLITLSSLSSKHNVYQGLKRSQHITNYWKDNIARQKSFLKDFSQQLKQYKRALENSDKAHTKPNLINFNKTFSILANLVNEIVIPLSNGAISFPNISKLEDGEESRHLIEFALNDYSDLVGSIGELKDAIATNGGYTPFAKVTLNHYTAEQKPHVFKNDIDAKIRELKLIELVEKLKNKTSKQIEEYFSKFDKFKIYDDRNISVIIRTQCFKYKPIPFLVKHELAKYIAEDEDAVLKVLDAIGATRSPAHDYAHNEDVFDLNHYPIKVAFDYAWEQLANSLYTTVTFPKEQCEKYLKDVFDCEVSKEPVFKFYADLLYIRKNLAVLEHKNNLPSNQEEFICKINNTFENIVLPYKISQFETYKKDILAWINDGRGHEKYTDAKQQLGFIRGGLKGRIKAQKMFRKDKYGKIKSYYENPYTKLTNEFKQISSTYGKTFAELRDKFKEKNNITKITHFGIIIEDKNRDRYLLANGLQHEQINHVSTILNKLDKSSEFITYQVKSLTSKTLIKLIKNHTTKKGAISPYADFHTSKTGFNKNEIEKNWDNYKREQVLVEYVKDCLTDSTMAKNQNWAEFGWNFEKCNSYEDIEHEIDQKSYILQKHTISKQSIASLVEGGCLLLPIINQDITSKERKDKNQFSKDWKQIFEGSKEFRLHPEFAVSYRTPIEGYPVQKRYGRLQFVCAFNCEIIPQNGEFINLKKQIENFNDEDAQKEAVEEFNKKVNHALSDKEYVVIGIDRGLKQLATLCVLDKRGKILGDFEIYKKEFVRAEKRSESHWEHTQAETRHILDLSNLRVETTIEGKKVLVDQSLTLVKKNRDTPDEEATEENKQKIKLKQLSYIRKLQHKMQTNEQDVLDLINNEPSDEEFKKRIEGLISSFGEGQKYADLPINTMREMISDLQGVIARGNNQTEKNKIIELDAADNLKQGIVANMIGIVNYIFAKYSYKAYISLEDLSRAYGGAKSGYDGRYLPSTSQDEDVDFKEQQNQMLAGLGTYQFFEMQLLKKLQKIQSDNTVLRFVPAFRSADNYRNILRLEETKYKSKPFGVVHFIDPKFTSKKCPVCSKTNVYRDKDDILVCKECGFRSDSQLKERENNIHYIHNGDDNGAYHIALKSVENLIQMK

SEQ ID NO: 2 - protein, 1231 aa, synthetic construct
  REGION 1..1231: note = BEC67 AA Sequence
MKSFENSCQISKTLRFGATLKEDKKKCKSHLELQEFVDISYKTLKSSATIAESLNEIELVKKCERCYSEIIKFHEAWGQIYCRTNQIAVYKDWYRQLARKARFDVGDQNSQLITLSSLSSKHNVYQGLKRSQHITNYWKDNIARQKSFLKDFSQQLKQYKRALENSDKAHTKPNLINFNKTFSILANLVNEIVIPLSNGAISFPNISKLEDGEESRHLIEFALNDYSDLVGSIGELKDAIATNGGYTPFAKVTLNHYTAEQKPHVFKNDIDAKIRELKLIELVEKLKNKTSKQIEEYFSKFDKFKIYDDRNISVIIRTQCFKYKPIPFLVKHELAKYIAEDEDAVLKVLDAIGATRSPAHDYAHNEDVFDLNHYPIKVAFDYAWEQLANSLYTTVTFPKEQCEKYLKDVFDCEVSKEPVFKFYADLLYIRKNLAVLEHKNNLPSNQEEFICKINNTFENIVLPYKISQFETYKKDILAWINDGRGHEKYTDSKRELGFIRGGLKGRIKAQKMFRKDKYGKIKSYYENPYTKLTEEFKNLAITYGKTFAELRDKFKEKNNITKITHFGIIIEDKNRDRYLLANGLQHEQINHVSTILNKLDKSSEFITYQVKSLTSKTLIKLIKNHTTKKGAISPYADFHTSKTGFNKNEIEKNWDNYKREQVLVEYVKDCLTDSTMAKNQNWAEFGWDLESCNSYEDIEHEIDQKSYILQKHTISKQSIASLVEGGCLLLPIINQDITSKERKDKNQFSKDWKQIFEGSKEFRLHPEFAVSYRTPIEGYPVQKRYGRLQFVCAFNCEIIPQNGEFINLKKQIENFNDEDAQKEAVEEFNKKVNHALSDKEYVVIGIDRGLKQLATLCVLDKRGKILGDFEIYKKEFVRAEKRSESHWEHTQAETRHILDLSNLRVETTIEGKKVLVDQSLTLVKKNRDTPDEEATEENKQKIKLKQLSYIRKLQHKMQTNEQDVLDLINNEPSDEEFKKRIEGLISSFGEGQKYADLPINTMREMISDLQGVIARGNNQTEKNKIIELDAADNLKQGIVANMIGIVNYIFAKYSYKAYISLEDLSRAYGGAKSGYDGRYLPSTSQDEDVDFKEQQNQMLAGLGTYQFFEMQLLKKLQKIQSDNTVLRFVPAFRSADNYRNILRLEETKYKSKPFGVVHFIDPKFTSKKCPVCSKTNVYRDKDDILVCKECGFRSDSQLKERENNIHYIHNGDDNGAYHIALKSVENLIQMK

SEQ ID NO: 3 - protein, 1231 aa, synthetic construct
  REGION 1..1231: note = BEC10 AA Sequence
MKSFENSCQISKTLRFGATLKEDKKKCKSHLELQEFVDISYKTLKSSATIAESLNEIELVKKCERCYSEIIKFHEAWGQIYCRTNQIAVYKDWYRQLARKARFDVGDQNSQLITLSSLSSKHNVYQGLKRSQHITNYWKDNIARQKSFLKDFSQQLKQYKRALENSDKAHTKPNLINFNKTFSILANLVNEIVIPLSNGAISFPNISKLEDGEESRHLIEFALNDYSDLVGSIGELKDAIATNGGYTPFAKVTLNHYTAEQKPHVFKNDIDAKIRELKLIELVEKLKNKTSKQIEEYFSKFDKFKIYDDRNISVIIRTQCFKYKPIPFLVKHELAKYIAEDEDAVLKVLDAIGATRSPAHDYAHNEDVFDLNHYPIKVAFDYAWEQLANGVYTTVTFPKEQCEKYLKDVFDCEVSKEPVFKFYADLLYIRKNLAVLEHKNNLPSNQEEFICKINNTFENIVLPYKIKEFETYKKDILAWINDGRGHEKYTDAKQQLGFIRGGLKGRIKAQKMFRKDKYGKIKSYYENPYTKLTNEFKQISSTYGKTFAELRDKFKEKNNITKITHFGIIIEDNNKDRYLLANGLQHEQINHVEAILGKLNTSSEFITYQVKSLTSKTLIKLIKNHTTKKGAISPYADFHTSKIHVDWTKIKKEWDNYKREQVLLQYVKDCLTDSTMAKNQNWAEFGWNFEKCNSYEDIEHEIDQKSYILQKHTISKQSIASLVEGGCLLLPIINQDITSQGRKDKNQFSKDWKQIFEGSKEFRLHPEFAVSYRTPIKDYPKDKRYGRLQFVCAFNCEIIPQNGEFINLKKQIENFNDEDAQKEAVEEFNKKVNHALLGKEYVVIGIDRGLKQLATLCVLDKRGKILGDFEIYKKEFVRAEKRSKNYWKHTLAETRHILDLSNLRVETTIEDNKVLVDQSLTLVKKNRDTPDEEATEENKQKIKLKQLSYIRKLQHKMQTNEQDVLDLINNEPSDEEFKKRIEGLISSFGEGQKYADLPINTMREMISDLQGVIARGNNQTEKNKIIELDAADNLKQGIVANMIGIVNYIFAKYSYKAYISLEDLSRAYGGAKSGYDGRYLPSTSQDEDVDFKEQQNQMLAGLGTYQFFEMQLLKKLQKIQSDNTVLRFVPAFRSADNYRNIIKLEETKYKSKPFGVVHFIDPKFTSKKCPVCSKTNVYRDKDDILVCKECGFRSDSQLKERENNIHYIHNGDDNGAYHIALKSVENLIQMK

SEQ ID NO: 4 - DNA, 3696 bp, synthetic construct
  misc_feature 1..3696: note = BEC85 NT Sequence (Yeast codon optimized)
  source 1..3696: mol_type = other DNA
atgaagtccttcgaaaactcctgccaaatttctaagactttgagattcggtgccactttgaaagaggataagaaaaagtgcaagtcccacttggaattgcaagaattcgttgacatttcctacaagaccttgaagtcatctgctaccattgctgaatccttgaacgaaatcgaattggtcaaaaagtgcgaaaggtgctactccgaaattatcaagttccatgaagcttggggtcaaatctactgtagaactaaccaaattgccgtctacaaggactggtatagacaattggctagaaaggccagattcgatgttggtgatcaaaactctcaactgatcaccttgtcatccttatcttctaagcacaacgtttaccagggtttgaagagatctcaacatattaccaactactggaaggacaacattgccagacaaaagtctttcctgaaggatttctctcaacaattgaagcagtacaagagagctttggaaaattctgataaggctcataccaagccaaacctgattaacttcaacaagaccttctccattttggccaacttggttaacgaaattgtcatcccattgtccaacggtgctatttcttttccaaacatctccaagttggaagatggtgaagaatccagacatttgattgaattcgctctgaacgattactccgatttggttggttctattggtgaattgaaggatgctattgctaccaatggtggttatactccatttgctaaggttaccttgaaccattacactgctgaacaaaagccacacgttttcaagaacgatattgatgccaagatccgtgagttgaagttgattgagttggtcgaaaagttgaagaacaagacgtccaagcaaatcgaagagtacttttccaagttcgacaagttcaagatctacgatgatagaaacatctccgttatcattagaacccaatgctttaagtacaagcccattccatttttggtcaagcacgaattggctaagtatatcgctgaagatgaagatgccgttttgaaggttttggatgcaattggtgctactagatctccagctcatgattacgctcataatgaagatgtttttgacttgaatcactacccaatcaaggttgcttttgattacgcatgggaacaattggctaactccttgtacactactgttactttcccaaaagaacagtgcgagaagtacttgaaggatgtttttgattgcgaagtctccaaagaaccagtttttaagttctacgccgacctgttgtacatcagaaagaatttggctgttttggagcacaagaacaacctgccatctaatcaagaggaattcatctgcaagattaacaacaccttcgagaacatcgttctgccatacaagatttctcaattcgaaacctacaagaaggatatcttggcctggattaacgatggtagaggtcacgaaaagtacaccgatgctaaacaacagttgggttttattagaggtggtctgaaaggtagaatcaaggctcaaaagatgttcagaaaagacaagtacggtaagatcaagtcctactacgaaaacccatacactaagttgaccaacgaattcaagcaaatctcttctacctacggtaaaaccttcgctgaattgagagacaagttcaaagaaaagaacaacatcaccaagatcacccatttcggtattatcatcgaagataagaacagggacagatacttgttggccaacggtttgcaacacgaacaaatcaaccatgtctccaccatcttgaacaagttggataagtcatccgaattcatcacctaccaagttaagtctttgacctctaagaccctgatcaagctgattaagaatcataccaccaagaagggtgcaatttctccatatgctgatttccatacttctaagaccggctttaacaaaaacgagatcgaaaagaactgggacaactacaagagagaacaagtcttggttgaatacgttaaggattgcttgaccgattctaccatggctaagaatcaaaattgggctgaatttggttggaacttcgagaagtgtaactcctacgaagatatcgaacacgaaatcgatcagaagtcttacatcttgcaaaagcataccatttccaagcaatctattgcttctttggttgaaggtggttgtttgttgttgccaattatcaaccaggacatcacctctaaagaacgtaaggacaagaatcagttctccaaggattggaagcaaatcttcgaaggttccaaagaattcagattgcatccagaattcgccgtttcttacagaactccaattgaaggttacccagtccaaaaaagatacggtagattgcaattcgtttgcgcttttaactgcgaaatcatcccacaaaatggtgagttcatcaacttgaagaagcagatcgagaacttcaacgacgaagatgctcaaaaagaagccgtcgaagagttcaacaaaaaggttaaccatgccttgtccgacaaagaatacgttgttattggtatcgacagaggcttgaagcaattagctactttgtgtgttttggacaagaggggtaagattttgggtgactttgaaatctacaaaaaagagttcgtcagagccgaaaagagatctgaatctcattgggaacatactcaagctgaaaccagacatatcttggacttgtctaacttgagagttgaaactaccattgagggcaaaaaggtgttggttgatcaatctttgacactggtcaagaagaacagagatactccagatgaagaagctaccgaagaaaacaagcagaagattaagttgaagcagctgtcctacattagaaagttgcaacacaagatgcagaccaatgaacaagatgtcttggacctgatcaacaacgaaccatctgatgaagaattcaaaaagaggatcgagggcttgatttcttcattcggtgaaggtcaaaagtacgctgatttgcctattaacaccatgagggaaatgatctctgacttgcaaggtgttattgccagaggaaacaatcagactgagaaaaacaagatcatcgaattggatgctgccgacaatttgaaacaaggtatcgttgctaacatgatcggtatcgttaactacattttcgccaagtactcctacaaggcctacatttctttagaagatttgtccagagcttacggtggtgcaaaatctggttatgatggtagatatttgccatccacctctcaagatgaggatgttgactttaaagaacagcagaatcaaatgttggctggcttgggtacttaccaattttttgaaatgcagctgctgaaaaagctgcagaagatccaatctgataacaccgttttgagatttgttccagcttttagatccgccgataactacagaaatatcttgagattggaagagactaagtacaagtctaagccattcggtgttgttcatttcatcgatccaaagttcacatctaagaagtgtccagtttgctctaagactaacgtttacagagataaggatgacatcctggtctgtaaagaatgtggttttaggtccgactcacagttgaaagaaagggaaaacaacatccactacatccacaatggtgatgataatggtgcttaccatattgccttgaagtccgtcgaaaacttgattcagatgaagtaa

SEQ ID NO: 5 - DNA, 3696 bp, synthetic construct
  misc_feature 1..3696: note = BEC67 NT Sequence (Yeast codon optimized)
  source 1..3696: mol_type = other DNA
atgaagtccttcgaaaactcctgccaaatttctaagactttgagattcggtgccactttgaaagaggataagaaaaagtgcaagtcccacttggaattgcaagaattcgttgacatttcctacaagaccttgaagtcatctgctaccattgctgaatccttgaacgaaatcgaattggtcaaaaagtgcgaaaggtgctactccgaaattatcaagttccatgaagcttggggtcaaatctactgtagaactaaccaaattgccgtctacaaggactggtatagacaattggctagaaaggccagattcgatgttggtgatcaaaactctcaactgatcaccttgtcatccttatcttctaagcacaacgtttaccagggtttgaagagatctcaacatattaccaactactggaaggacaacattgccagacaaaagtctttcctgaaggatttctctcaacaattgaagcagtacaagagagctttggaaaattctgataaggctcataccaagccaaacctgattaacttcaacaagaccttctccattttggccaacttggttaacgaaattgtcatcccattgtccaacggtgctatttcttttccaaacatctccaagttggaagatggtgaagaatccagacatttgattgaattcgctctgaacgattactccgatttggttggttctattggtgaattgaaggatgctattgctaccaatggtggttatactccatttgctaaggttaccttgaaccattacactgctgaacaaaagccacacgttttcaagaacgatattgatgccaagatccgtgagttgaagttgattgagttggtcgaaaagttgaagaacaagacgtccaagcaaatcgaagagtacttttccaagttcgacaagttcaagatctacgatgatagaaacatctccgttatcattagaacccaatgctttaagtacaagcccattccatttttggtcaagcacgaattggctaagtatatcgctgaagatgaagatgccgttttgaaggttttggatgcaattggtgctactagatctccagctcatgattacgctcataatgaagatgtttttgacttgaatcactacccaatcaaggttgcttttgattacgcatgggaacaattggctaactccttgtacactactgttactttcccaaaagaacagtgcgagaagtacttgaaggatgtttttgattgcgaagtctccaaagaaccagtttttaagttctacgccgacctgttgtacatcagaaagaatttggctgttttggagcacaagaacaacctgccatctaatcaagaggaattcatctgcaagattaacaacaccttcgagaacatcgttctgccatacaagatttctcaattcgaaacctacaagaaggatatcttggcctggattaacgatggtagaggtcacgaaaagtacaccgattctaaaagagaattgggttttattagaggtggtctgaaaggtagaatcaaggctcaaaagatgttcagaaaagacaagtacggtaagatcaagtcctactacgaaaacccatacactaagttgaccgaagagttcaagaacttggccatcacctacggtaaaaccttcgctgaattgagagacaagttcaaagaaaagaacaacatcaccaagatcacccatttcggtattatcatcgaagataagaacagggacagatacttgttggccaacggtttgcaacacgaacaaatcaaccatgtctccaccatcttgaacaagttggataagtcatccgaattcatcacctaccaagttaagtctttgacctctaagaccctgatcaagctgattaagaatcataccaccaagaagggtgcaatttctccatatgctgatttccatacttctaagaccggctttaacaaaaacgagatcgaaaagaactgggacaactacaagagagaacaagtcttggttgaatacgttaaggattgcttgaccgattctaccatggctaagaatcaaaattgggctgaatttggttgggatttggagtcttgtaactcctacgaagatatcgaacacgaaatcgatcagaagtcttacatcttgcaaaagcataccatttccaagcaatctattgcttctttggttgaaggtggttgtttgttgttgccaattatcaaccaggacatcacctctaaagaacgtaaggacaagaatcagttctccaaggattggaagcaaatcttcgaaggttccaaagaattcagattgcatccagaattcgccgtttcttacagaactccaattgaaggttacccagtccaaaaaagatacggtagattgcaattcgtttgcgcttttaactgcgaaatcatcccacaaaatggtgagttcatcaacttgaagaagcagatcgagaacttcaacgacgaagatgctcaaaaagaagccgtcgaagagttcaacaaaaaggttaaccatgccttgtccgacaaagaatacgttgttattggtatcgacagaggcttgaagcaattagctactttgtgtgttttggacaagaggggtaagattttgggtgactttgaaatctacaaaaaagagttcgtcagagccgaaaagagatctgaatctcattgggaacatactcaagctgaaaccagacatatcttggacttgtctaacttgagagttgaaactaccattgagggcaaaaaggtgttggttgatcaatctttgacactggtcaagaagaacagagatactccagatgaagaagctaccgaagaaaacaagcagaagattaagttgaagcagctgtcctacattagaaagttgcaacacaagatgcagaccaatgaacaagatgtcttggacctgatcaacaacgaaccatctgatgaagaattcaaaaagaggatcgagggcttgatttcttcattcggtgaaggtcaaaagtacgctgatttgcctattaacaccatgagggaaatgatctctgacttgcaaggtgttattgccagaggaaacaatcagactgagaaaaacaagatcatcgaattggatgctgccgacaatttgaaacaaggtatcgttgctaacatgatcggtatcgttaactacattttcgccaagtactcctacaaggcctacatttctttagaagatttgtccagagcttacggtggtgcaaaatctggttatgatggtagatatttgccatccacctctcaagatgaggatgttgactttaaagaacagcagaatcaaatgttggctggcttgggtacttaccaattttttgaaatgcagctgctgaaaaagctgcagaagatccaatctgataacaccgttttgagatttgttccagcttttagatccgccgataactacagaaatatcttgagattggaagagactaagtacaagtctaagccattcggtgttgttcatttcatcgatccaaagttcacatctaagaagtgtccagtttgctctaagactaacgtttacagagataaggatgacatcctggtctgtaaagaatgtggttttaggtccgactcacagttgaaagaaagggaaaacaacatccactacatccacaatggtgatgataatggtgcttaccatattgccttgaagtccgtcgaaaacttgattcagatgaagtaa

SEQ ID NO: 6 - DNA, 3696 bp, synthetic construct
  misc_feature 1..3696: note = BEC10 NT Sequence (Yeast codon optimized)
  source 1..3696: mol_type = other DNA
atgaagtccttcgaaaactcctgccaaatttctaagactttgagattcggtgccactttgaaagaggataagaaaaagtgcaagtcccacttggaattgcaagaattcgttgacatttcctacaagaccttgaagtcatctgctaccattgctgaatccttgaacgaaatcgaattggtcaaaaagtgcgaaaggtgctactccgaaattatcaagttccatgaagcttggggtcaaatctactgtagaactaaccaaattgccgtctacaaggactggtatagacaattggctagaaaggccagattcgatgttggtgatcaaaactctcaactgatcaccttgtcatccttatcttctaagcacaacgtttaccagggtttgaagagatctcaacatattaccaactactggaaggacaacattgccagacaaaagtctttcctgaaggatttctctcaacaattgaagcagtacaagagagctttggaaaattctgataaggctcataccaagccaaacctgattaacttcaacaagaccttctccattttggccaacttggttaacgaaattgtcatcccattgtccaacggtgctatttcttttccaaacatctccaagttggaagatggtgaagaatccagacatttgattgaattcgctctgaacgattactccgatttggttggttctattggtgaattgaaggatgctattgctaccaatggtggttatactccatttgctaaggttaccttgaaccattacactgctgaacaaaagccacacgttttcaagaacgatattgatgccaagatccgtgagttgaagttgattgagttggtcgaaaagttgaagaacaagacgtccaagcaaatcgaagagtacttttccaagttcgacaagttcaagatctacgatgatagaaacatctccgttatcattagaacccaatgctttaagtacaagcccattccatttttggtcaagcacgaattggctaagtatatcgctgaagatgaagatgccgttttgaaggttttggatgcaattggtgctactagatctccagctcatgattacgctcataatgaagatgtttttgacttgaatcactacccaatcaaggttgcttttgattacgcatgggaacaattggctaacggtgtctacactactgttactttcccaaaagaacagtgcgagaagtacttgaaggatgtttttgattgcgaagtctccaaagaaccagtttttaagttctacgccgacctgttgtacatcagaaagaatttggctgttttggagcacaagaacaacctgccatctaatcaagaggaattcatctgcaagattaacaacaccttcgagaacatcgttctgccatacaagattaaagagttcgaaacctacaagaaggatatcttggcctggattaacgatggtagaggtcacgaaaagtacaccgatgctaaacaacagttgggttttattagaggtggtctgaaaggtagaatcaaggctcaaaagatgttcagaaaagacaagtacggtaagatcaagtcctactacgaaaacccatacactaagttgaccaacgaattcaagcaaatctcttctacctacggtaaaaccttcgctgaattgagagacaagttcaaagaaaagaacaacatcaccaagatcacccatttcggtattatcatcgaagataacaacaaagacagatacttgttggccaacggtttgcaacacgaacaaatcaaccatgtcgaagccattttgggtaagttgaacacctcatccgaattcatcacctaccaagttaagtctttgacctctaagaccctgatcaagctgattaagaatcataccaccaagaagggtgcaatttctccatatgctgatttccatacttctaagatccatgttgattggaccaagatcaagaaagaatgggacaactacaagagagaacaagtcttgttgcaatacgttaaggattgcttgaccgattctaccatggctaagaatcaaaattgggctgaatttggttggaacttcgagaagtgtaactcctacgaagatatcgaacacgaaatcgatcagaagtcttacatcttgcaaaagcataccatttccaagcaatctattgcttctttggttgaaggtggttgtttgttgttgccaattatcaaccaggacatcacctctcaaggtcgtaaggacaagaatcagttctccaaggattggaagcaaatcttcgaaggttccaaagaattcagattgcatccagaattcgccgtttcttacagaactccaattaaggactacccaaaggataaaagatacggtagattgcaattcgtttgcgcttttaactgcgaaatcatcccacaaaatggtgagttcatcaacttgaagaagcagatcgagaacttcaacgacgaagatgctcaaaaagaagccgtcgaagagttcaacaaaaaggttaaccatgccttgttgggtaaagaatacgttgttattggtatcgacagaggcttgaagcaattagctactttgtgtgttttggacaagaggggtaagattttgggtgactttgaaatctacaaaaaagagttcgtcagagccgaaaagagatctaagaattactggaagcacaccttggctgaaaccagacatatcttggacttgtctaacttgagagttgaaactaccattgaggataataaggtgttggttgatcaatctttgacactggtcaagaagaacagagatactccagatgaagaagctaccgaagaaaacaagcagaagattaagttgaagcagctgtcctacattagaaagttgcaacacaagatgcagaccaatgaacaagatgtcttggacctgatcaacaacgaaccatctgatgaagaattcaaaaagaggatcgagggcttgatttcttcattcggtgaaggtcaaaagtacgctgatttgcctattaacaccatgagggaaatgatctctgacttgcaaggtgttattgccagaggaaacaatcagactgagaaaaacaagatcatcgaattggatgctgccgacaatttgaaacaaggtatcgttgctaacatgatcggtatcgttaactacattttcgccaagtactcctacaaggcctacatttctttagaagatttgtccagagcttacggtggtgcaaaatctggttatgatggtagatatttgccatccacctctcaagatgaggatgttgactttaaagaacagcagaatcaaatgttggctggcttgggtacttaccaattttttgaaatgcagctgctgaaaaagctgcagaagatccaatctgataacaccgttttgagatttgttccagcttttagatccgccgataactacagaaatatcatcaagttggaagagactaagtacaagtctaagccattcggtgttgttcatttcatcgatccaaagttcacatctaagaagtgtccagtttgctctaagactaacgtttacagagataaggatgacatcctggtctgtaaagaatgtggttttaggtccgactcacagttgaaagaaagggaaaacaacatccactacatccacaatggtgatgataatggtgcttaccatattgccttgaagtccgtcgaaaacttgattcagatgaagtaa

SEQ ID NO: 7 - DNA, 3696 bp, synthetic construct
  misc_feature 1..3696: note = BEC10 NT Sequence (E. coli codon optimized)
  source 1..3696: mol_type = other DNA
atgaaaagctttgaaaacagctgccagattagcaaaaccctgcgttttggtgcaaccctgaaagaggataaaaagaaatgtaaaagccacctggaactgcaagaatttgtggatattagctacaagaccctgaaaagcagcgcaaccattgcagaaagcctgaatgaaattgagctggtgaaaaaatgcgaacgctgctatagcgaaatcatcaaatttcatgaagcctggggtcagatttattgtcgtaccaatcagattgccgtgtacaaagattggtatcgtcagctggcacgtaaagcacgttttgatgttggtgatcagaatagccagctgattaccctgagcagcctgagtagcaaacataatgtttatcagggtctgaaacgcagccagcatattaccaattattggaaagataacattgcccgtcagaaaagcttcctgaaagattttagccagcagctgaaacagtataaacgtgcactggaaaatagcgataaagcacataccaaaccgaacctgatcaacttcaataaaacctttagcattctggccaacctggtgaacgaaattgttattccgctgagcaatggtgcaattagctttccgaatatcagcaaactggaagatggtgaagaaagccgtcatctgattgaatttgccctgaatgattatagcgatctggttggtagcattggcgaactgaaagatgcaattgccaccaatggtggttataccccgtttgcaaaagttaccctgaaccattataccgcagaacagaaaccgcatgtgttcaaaaatgatatcgatgccaaaatccgtgagctgaaactgattgaactggttgagaaactgaaaaacaaaaccagcaagcagatcgaagagtacttcagcaaattcgacaagttcaaaatctatgatgatcgcaacatcagcgtgattattcgtacccagtgctttaaatacaaaccgattccgtttctggtgaaacatgaactggccaaatatatcgccgaagatgaagatgcagttctgaaagttctggatgccattggtgcgacccgtagtccggcacatgattatgcacataatgaagatgtgttcgatctgaaccactatccgattaaagtggcatttgattatgcctgggaacagctggcaaatggtgtgtataccaccgtgacctttccgaaagaacagtgtgaaaaatatctgaaggatgtgtttgattgcgaggttagcaaagaaccggtgtttaaattctatgccgatctgctgtatatccgcaaaaatctggccgttctggaacataaaaacaatctgccgagcaaccaagaagagttcatttgcaaaatcaacaacaccttcgaaaacatcgtgctgccgtataaaatcaaagagttcgaaacctacaaaaaagacatcctggcctggattaatgatggtcgtggtcatgagaaatataccgatgcaaaacagcagctgggttttattcgtggtggtctgaaaggtcgtattaaagcccagaaaatgttccgcaaagataagtacggtaagatcaaaagctattatgagaacccgtataccaaactgaccaacgaatttaagcagatcagcagcacctatggcaaaacctttgccgaactgcgtgataagtttaaagagaagaacaacatcaccaaaatcacccactttggcatcatcatcgaggataataacaaagatcgttatctgctggccaatggtctgcagcatgagcagattaatcatgttgaagcaattctgggcaaactgaataccagcagcgaatttatcacctatcaggttaaaagcctgaccagtaaaaccctgattaagctgattaaaaaccataccaccaaaaagggtgcgattagcccgtatgcagattttcataccagcaaaattcatgtggactggacgaaaatcaaaaaagagtgggataactataagcgcgaacaggttctgctgcagtatgttaaagattgtctgaccgatagcaccatggccaaaaatcagaattgggcagaatttggctggaactttgaaaaatgcaacagctatgaagatatcgagcacgagattgaccagaaaagttatatcctgcagaaacacaccatctccaaacagagcattgcaagcctggttgaaggtggttgtctgctgctgccgattattaaccaggatattacctcacagggtcgcaaagacaaaaaccagtttagtaaagattggaagcagatctttgaaggcagcaaagaatttcgtctgcatccggaatttgcagttagctatcgtaccccgatcaaagattatccgaaagataaacgttatggtcgcctgcagtttgtttgtgcctttaactgtgaaatcattccgcagaatggcgaattcatcaatctgaaaaagcagattgagaacttcaacgatgaggatgcacagaaagaagccgttgaagaattcaacaaaaaagtgaatcatgccctgctgggcaaagaatatgttgtgattggtattgatcgtggcctgaaacaactggcaaccctgtgtgttctggataaacgtggtaaaatcctgggcgattttgagatctacaagaaagaattcgttcgtgccgaaaaacgcagcaaaaactattggaaacacacactggcagaaacccgtcatattctggatctgagcaatctgcgtgttgaaaccacaatcgaagataacaaagtgctggttgatcagagtctgaccctggttaaaaagaatcgtgatacaccggatgaagaagccaccgaagaaaataaacagaagatcaaactgaagcagctgagctatattcgtaaactgcagcataaaatgcagaccaatgaacaggatgttctggacctgattaataacgaaccgagcgacgaagaatttaagaaacgtattgaaggtctgattagcagctttggtgaaggtcagaaatatgcggatctgcctattaataccatgcgcgaaatgattagtgatctgcagggtgttattgcccgtggtaataatcagacagagaaaaacaagatcatcgaactggatgcagccgataatctgaaacagggtattgttgcaaacatgattggcatcgtgaactacatctttgccaaatacagctacaaagcctatatcagcctggaagatctgagccgtgcatatggtggtgcaaaaagcggttatgatggacgctatctgccgtcaaccagccaggacgaagatgtggattttaaagaacagcagaatcagatgctggcaggtctgggtacatatcagttttttgaaatgcagctgctgaagaagctgcagaaaattcagagcgataataccgttctgcgctttgttccggcatttcgtagcgcagataactatcgcaatattatcaagctggaagaaaccaaatacaaaagcaaaccgtttggcgtggtgcactttattgatccgaaatttacgagcaaaaaatgtccggtttgcagcaaaaccaatgtgtatcgtgataaagatgacattctggtgtgtaaagaatgcggttttcgtagtgatagtcagctgaaagaacgcgaaaacaacatccactatattcataatggtgatgacaacggtgcctatcatattgcactgaaaagcgtggaaaatctgatccagatgaaataa

SEQ ID NO: 8 - DNA, 1191 bp, synthetic construct
  misc_feature 1..1191: note = araC-ParaBAD inducible promotor system
  source 1..1191: mol_type = other DNA
tatggagaaacagtagagagttgcgataaaaagcgtcaggtaggatccgctaatcttatggataaaaatgctatggcatagcaaagtgtgacgccgtgcaaataatcaatgtggacttttctgccgtgattatagacacttttgttacgcgtttttgtcatggctttggtcccgctttgttacagaatgcttttaataagcggggttaccggtttggttagcgagaagagccagtaaaagacgcagtgacggcaatgtctgatgcaatatggacaattggtttcttctctgaatggcgggagtatgaaaagtatggctgaagcgcaaaatgatcccctgctgccgggatactcgtttaatgcccatctggtggcgggtttaacgccgattgaggccaacggttatctcgatttttttatcgaccgaccgctgggaatgaaaggttatattctcaatctcaccattcgcggtcagggggtggtgaaaaatcagggacgagaatttgtttgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacggggttctttcgcccggatgaagcgcaccagccgcatttcagcgacctgtttgggcaaatcattaacgccgggcaaggggaagggcgctattcggagctgctggcgataaatctgcttgagcaattgttactgcggcgcatggaagcgattaacgagtcgctccatccaccgatggataatcgggtacgcgaggcttgtcagtacatcagcgatcacctggcagacagcaattttgatatcgccagcgtcgcacagcatgtttgcttgtcgccgtcgcgtctgtcacatcttttccgccagcagttagggattagcgtcttaagctggcgcgaggaccaacgtatcagccaggcgaagctgcttttgagcaccacccggatgcctatcgccaccgtcggtcgcaatgttggttttgacgatcaactctatttctcgcgggtatttaaaaaatgcaccggggccagcccgagcgagttccgtgccggttgtgaagaaaaagtgaatgatgtagccgtcaagttgtcataa

SEQ ID NO: 9 - DNA, 177 bp, synthetic construct
  misc_feature 1..177: note = fdT expression terminator
  source 1..177: mol_type = other DNA
aattttctgtatgaggttttgctaaacaactttcaacagtttcagcggagtgagaatagaaaggaacaactaaaggaattgcgaataataattttttcacgttgaaaatctccaaaaaaaaaggctccaaaaggagcctttaattgtatcggtttatcagcttgctttcgaggtgaa

SEQ ID NO: 10 - DNA, 10026 bp, synthetic construct
  misc_feature 1..10026: note = Nucleotide sequence of the CRISPR/BEC10-Ec vector system
  source 1..10026: mol_type = other DNA
ctaccggcgcggcagcgtgacccgtgtcggcggctccaacggctcgccatcgtccagaaaacacggctcatcgggcatcggcaggcgctgctgcccgcgccgttcccattcctccgtttcggtcaaggctggcaggtctggttccatgcccggaatgccgggctggctgggcggctcctcgccggggccggtcggtagttgctgctcgcccggatacagggtcgggatgcggcgcaggtcgccatgccccaacagcgattcgtcctggtcgtcgtgatcaaccaccacggcggcactgaacaccgacaggcgcaactggtcgcggggctggccccacgccacgcggtcattgaccacgtaggccgacacggtgccggggccgttgagcttcacgacggagatccagcgctcggccaccaagtccttgactgcgtattggaccgtccgcaaagaacgtccgatgagcttggaaagtgtcttttggctgaccaccacggcgttctggtggcccatctgcgccacgaggtgatgcagcagcattgccgccgtgggtttcctcgcaataagcccggcccacgcctcatgcgctttgcgttccgtttgcacccagtgaccgggcttgttcttggcttgaatgccgatttctctggactgcgtggccatgcttatctccatgcggtagggtgccgcacggttgcggcaccatgcgcaatcagctgcaacttttcggcagcgcgacaacaattatgcgttgcgtaaaagtggcagtcaattacagattttctttaacctacgcaatgagctattgcggggggtgccgcaatgagctgttgcgtaccccccttttttaagttgttgatttttaagtctttcgcatttcgccctatatctagttctttggtgcccaaagaagggcacccctgcggggttcccccacgccttcggcgcggctccccctccggcaaaaagtggcccctccggggcttgttgatcgactgcgcggccttcggccttgcccaaggtggcgctgcccccttggaacccccgcactcgccgccgtgaggctcggggggcaggcgggcgggcttcgccttcgactgcccccactcgcataggcttgggtcgttccaggcgcgtcaaggccaagccgctgcgcggtcgctgcgcgagccttgacccgccttccacttggtgtccaaccggcaagcgaagcgcgcaggccgcaggccggaggcttttccccagagaaaattaaaaaaattgatggggcaaggccgcaggccgcgcagttggagccggtgggtatgtggtcgaaggctgggtagccggtgggcaatccctgtggtcaagctcgtgggcaggcgcagcctgtccatcagcttgtccagcagggttgtccacgggccgagcgaagcgagccagccggtggccgctcgcggccatcgtccacatatccacgggctggcaagggagcgcagcgaccgcgcagggcgaagcccggagagcaagcccgtagggcgccgcagccgccgtaggcggtcacgactttgcgaagcaaagtctagtgagtatactcaagcattgagtggcccgccggaggcaccgccttgcgctgcccccgtcgagccggttggacaccaaaagggaggggcaggcatggcggcatacgcgatcatgcgatgcaagaagctggcgaaaatgggcaacgtggcggccagtctcaagcacgcctaccgcgagcgcgagacgcccaacgctgacgccagcaggacgccagagaacgagcactgggcggccagcagcaccgatgaagcgatgggccgactgcgcgagttgctgccagagaagcggcgcaaggacgctgtgttggcggtcgagtacgtcatgacggccagcccggaatggtggaagtcggccagccaagaacagcaggcggcgttcttcgagaaggcgcacaagtggctggcggacaagtacggggcggatcgcatcgtgacggccagcatccaccgtgacgaaaccagcccgcacatgaccgcgttcgtggtgccgctgacgcaggacggcaggctgtcggccaaggagttcatcggcaacaaagcgcagatgacccgcgaccagaccacgtttgcggccgctgtggccgatctagggctgcaacggggcatcgagggcagcaaggcacgtcacacgcgcattcaggcgttctacgaggccctggagcggccaccagtgggccacgtcaccatcagcccgcaagcggtcgagccacgcgcctatgcaccgcagggattggccgaaaagctgggaatctcaaagcgcgttgagacgccggaagccgtggccgaccggctgacaaaagcggttcggcaggggtatgagcctgccctacaggccgccgcaggagcgcgtgagatgcgcaagaaggccgatcaagcccaagagacggcccgagaccttcgggagcgcctgaagcccgttctggacgccctggggccgttgaatcgggatatgcaggccaaggccgccgcgatcatcaaggccgtgggcgaaaagctgctgacggaacagcgggaagtccagcgccagaaacaggcccagcgccagcaggaacgcgggcgcgcacatttccccgaaaagtgccacctgcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctagctgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttggtaccgggccccccctcgagtttattttaggaggcaaaaatgaaaagctttgaaaacagctgccagattagcaaaaccctgcgttttggtgcaaccctgaaagaggataaaaagaaatgtaaaagccacctggaactgcaagaatttgtggatattagctacaagaccctgaaaagcagcgcaaccattgcagaaagcctgaatgaaattgagctggtgaaaaaatgcgaacgctgctatagcgaaatcatcaaatttcatgaagcctggggtcagatttattgtcgtaccaatcagattgccgtgtacaaagattggtatcgtcagctggcacgtaaagcacgttttgatgttggtgatcagaatagccagctgattaccctgagcagcctgagtagcaaacataatgtttatcagggtctgaaacgcagccagcatattaccaattattggaaagataacattgcccgtcagaaaagcttcctgaaagattttagccagcagctgaaacagtataaacgtgcactggaaaatagcgataaagcacataccaaaccgaacctgatcaacttcaataaaacctttagcattctggccaacctggtgaacgaaattgttattccgctgagcaatggtgcaattagctttccgaatatcagcaaactggaagatggtgaagaaagccgtcatctgattgaatttgccctgaatgattatagcgatctggttggtagcattggcgaactgaaagatgcaattgccaccaatggtggttataccccgtttgcaaaagttaccctgaaccattataccgcagaacagaaaccgcatgtgttcaaaaatgatatcgatgccaaaatccgtgagctgaaactgattgaactggttgagaaactgaaaaacaaaaccagcaagcagatcgaagagtacttcagcaaattcgacaagttcaaaatctatgatgatcgcaacatcagcgtgattattcgtacccagtgctttaaatacaaaccgattccgtttctggtgaaacatgaactggccaaatatatcgccgaagatgaagatgcagttctgaaagttctggatgccattggtgcgacccgtagtccggcacatgattatgcacataatgaagatgtgttcgatctgaaccactatccgattaaagtggcatttgattatgcctgggaacagctggcaaatggtgtgtataccaccgtgacctttccgaaagaacagtgtgaaaaatatctgaaggatgtgtttgattgcgaggttagcaaagaaccggtgtttaaattctatgccgatctgctgtatatccgcaaaaatctggccgttctggaacataaaaacaatctgccgagcaaccaagaagagttcatttgcaaaatcaacaacaccttcgaaaacatcgtgctgccgtataaaatcaaagagttcgaaacctacaaaaaagacatcctggcctggattaatgatggtcgtggtcatgagaaatataccgatgcaaaacagcagctgggttttattcgtggtggtctgaaaggtcgtattaaagcccagaaaatgttccgcaaagataagtacggtaagatcaaaagctattatgagaacccgtataccaaactgaccaacgaatttaagcagatcagcagcacctatggcaaaacctttgccgaactgcgtgataagtttaaagagaagaacaacatcaccaaaatcacccactttggcatcatcatcgaggataataacaaagatcgttatctgctggccaatggtctgcagcatgagcagattaatcatgttgaagcaattctgggcaaactgaataccagcagcgaatttatcacctatcaggttaaaagcctgaccagtaaaaccctgattaagctgattaaaaaccataccaccaaaaagggtgcgattagcccgtatgcagattttcataccagcaaaattcatgtggactggacgaaaatcaaaaaagagtgggataactataagcgcgaacaggttctgctgcagtatgttaaagattgtctgaccgatagcaccatggccaaaaatcagaattgggcagaatttggctggaactttgaaaaatgcaacagctatgaagatatcgagcacgagattgaccagaaaagttatatcctgcagaaacacaccatctccaaacagagcattgcaagcctggttgaaggtggttgtctgctgctgccgattattaaccaggatattacctcacagggtcgcaaagacaaaaaccagtttagtaaagattggaagcagatctttgaaggcagcaaagaatttcgtctgcatccggaatttgcagttagctatcgtaccccgatcaaagattatccgaaagataaacgttatggtcgcctgcagtttgtttgtgcctttaactgtgaaatcattccgcagaatggcgaattcatcaatctgaaaaagcagattgagaacttcaacgatgaggatgcacagaaagaagccgttgaagaattcaacaaaaaagtgaatcatgccctgctgggcaaagaatatgttgtgattggtattgatcgtggcctgaaacaactggcaaccctgtgtgttctggataaacgtggtaaaatcctgggcgattttgagatctacaagaaagaattcgttcgtgccgaaaaacgcagcaaaaactattggaaacacacactggcagaaacccgtcatattctggatctgagcaatctgcgtgttgaaaccacaatcgaagataacaaagtgctggttgatcagagtctgaccctggttaaaaagaatcgtgatacaccggatgaagaagccaccgaagaaaataaacagaagatcaaactgaagcagctgagctatattcgtaaactgcagcataaaatgcagaccaatgaacaggatgttctggacctgattaataacgaaccgagcgacgaagaatttaagaaacgtattgaaggtctgattagcagctttggtgaaggtcagaaatatgcggatctgcctattaataccatgcgcgaaatgattagtgatctgcagggtgttattgcccgtggtaataatcagacagagaaaaacaagatcatcgaactggatgcagccgataatctgaaacagggtattgttgcaaacatgattggcatcgtgaactacatctttgccaaatacagctacaaagcctatatcagcctggaagatctgagccgtgcatatggtggtgcaaaaagcggttatgatggacgctatctgccgtcaaccagccaggacgaagatgtggattttaaagaacagcagaatcagatgctggcaggtctgggtacatatcagttttttgaaatgcagctgctgaagaagctgcagaaaattcagagcgataataccgttctgcgctttgttccggcatttcgtagcgcagataactatcgcaatattatcaagctggaagaaaccaaatacaaaagcaaaccgtttggcgtggtgcactttattgatccgaaatttacgagcaaaaaatgtccggtttgcagcaaaaccaatgtgtatcgtgataaagatgacattctggtgtgtaaagaatgcggttttcgtagtgatagtcagctgaaagaacgcgaaaacaacatccactatattcataatggtgatgacaacggtgcctatcatattgcactgaaaagcgtggaaaatctgatccagatgaaataatggtctagaggtcgaaattcaaattgtgagcggataacaatttgaattttctgtatgaggttttgctaaacaactttcaacagtttcagcggagtgagaatagaaaggaacaactaaaggaattgcgaataataattttttcacgttgaaaatctccaaaaaaaaaggctccaaaaggagcctttaattgtatcggtttatcagcttgctttcgaggtgaatttcgaccctctagcgaaaatgcaagagcaaagacgaaaacatgccacacatgaggaataccgattctctcattaacatattcaggccagttatctgggcttaaaagcagaagtccaacccagataacgatcatatacatggttctctccagaggttcattactgaacactcgtccgagaataacgagtggatcccctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggtgaatccgttagcgaggtgccgccggcttccattcaggtcgaggtggcccggctccatgcaccgcgacgcaacgcggggaggcagacaaggtatagggcggcgaggcggctacagccgatagtctggaacagcgcacttacgggttgctgcgcaacccaagtg

SEQ ID NO: 11 - DNA, 10225 bp, synthetic construct
  misc_feature 1..10225: note = Nucleotide sequence of the CRISPR/FnCpf1-Ec vector system
  source 1..10225: mol_type = other DNA
ctaccggcgcggcagcgtgacccgtgtcggcggctccaacggctcgccatcgtccagaaaacacggctcatcgggcatcggcaggcgctgctgcccgcgccgttcccattcctccgtttcggtcaaggctggcaggtctggttccatgcccggaatgccgggctggctgggcggctcctcgccggggccggtcggtagttgctgctcgcccggatacagggtcgggatgcggcgcaggtcgccatgccccaacagcgattcgtcctggtcgtcgtgatcaaccaccacggcggcactgaacaccgacaggcgcaactggtcgcggggctggccccacgccacgcggtcattgaccacgtaggccgacacggtgccggggccgttgagcttcacgacggagatccagcgctcggccaccaagtccttgactgcgtattggaccgtccgcaaagaacgtccgatgagcttggaaagtgtcttttggctgaccaccacggcgttctggtggcccatctgcgccacgaggtgatgcagcagcattgccgccgtgggtttcctcgcaataagcccggcccacgcctcatgcgctttgcgttccgtttgcacccagtgaccgggcttgttcttggcttgaatgccgatttctctggactgcgtggccatgcttatctccatgcggtagggtgccgcacggttgcggcaccatgcgcaatcagctgcaacttttcggcagcgcgacaacaattatgcgttgcgtaaaagtggcagtcaattacagattttctttaacctacgcaatgagctattgcggggggtgccgcaatgagctgttgcgtaccccccttttttaagttgttgatttttaagtctttcgcatttcgccctatatctagttctttggtgcccaaagaagggcacccctgcggggttcccccacgccttcggcgcggctccccctccggcaaaaagtggcccctccggggcttgttgatcgactgcgcggccttcggccttgcccaaggtggcgctgcccccttggaacccccgcactcgccgccgtgaggctcggggggcaggcgggcgggcttcgccttcgactgcccccactcgcataggcttgggtcgttccaggcgcgtcaaggccaagccgctgcgcggtcgctgcgcgagccttgacccgccttccacttggtgtccaaccggcaagcgaagcgcgcaggccgcaggccggaggcttttccccagagaaaattaaaaaaattgatggggcaaggccgcaggccgcgcagttggagccggtgggtatgtggtcgaaggctgggtagccggtgggcaatccctgtggtcaagctcgtgggcaggcgcagcctgtccatcagcttgtccagcagggttgtccacgggccgagcgaagcgagccagccggtggccgctcgcggccatcgtccacatatccacgggctggcaagggagcgcagcgaccgcgcagggcgaagcccggagagcaagcccgtagggcgccgcagccgccgtaggcggtcacgactttgcgaagcaaagtctagtgagtatactcaagcattgagtggcccgccggaggcaccgccttgcgctgcccccgtcgagccggttggacaccaaaagggaggggcaggcatggcggcatacgcgatcatgcgatgcaagaagctggcgaaaatgggcaacgtggcggccagtctcaagcacgcctaccgcgagcgcgagacgcccaacgctgacgccagcaggacgccagagaacgagcactgggcggccagcagcaccgatgaagcgatgggccgactgcgcgagttgctgccagagaagcggcgcaaggacgctgtgttggcggtcgagtacgtcatgacggccagcccggaatggtggaagtcggccagccaagaacagcaggcggcgttcttcgagaaggcgcacaagtggctggcggacaagtacggggcggatcgcatcgtgacggccagcatccaccgtgacgaaaccagcccgcacatgaccgcgttcgtggtgccgctgacgcaggacggcaggctgtcggccaaggagttcatcggcaacaaagcgcagatgacccgcgaccagaccacgtttgcggccgctgtggccgatctagggctgcaacggggcatcgagggcagcaaggcacgtcacacgcgcattcaggcgttctacgaggccctggagcggccaccagtgggccacgtcaccatcagcccgcaagcggtcgagccacgcgcctatgcaccgcagggattggccgaaaagctgggaatctcaaagcgcgttgagacgccggaagccgtggccgaccggctgacaaaagcggttcggcaggggtatgagcctgccctacaggccgccgcaggagcgcgtgagatgcgcaagaaggccgatcaagcccaagagacggcccgagaccttcgggagcgcctgaagcccgttctggacgccctggggccgttgaatcgggatatgcaggccaaggccgccgcgatcatcaaggccgtgggcgaaaagctgctgacggaacagcgggaagtccagcgccagaaacaggcccagcgccagcaggaacgcgggcgcgcacatttccccgaaaagtgccacctgcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctagctgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttggtaccgggccccccctcgagtttattttaggaggcaaaaatgtcaatttatcaagaatttgttaataaatatagtttaagtaaaactctaagatttgagttaatcccacagggtaaaacacttgaaaacataaaagcaagaggtttgattttagatgatgagaaaagagctaaagactacaaaaaggctaaacaaataattgataaatatcatcagttttttatagaggagatattaagttcggtttgtattagcgaagatttattacaaaactattctgatgtttattttaaacttaaaaagagtgatgatgataatctacaaaaagattttaaaagtgcaaaagatacgataaagaaacaaatatctgaatatataaaggactcagagaaatttaagaatttgtttaatcaaaaccttatcgatgctaaaaaagggcaagagtcagatttaattctatggctaaagcaatctaaggataatggtatagaactatttaaagccaatagtgatatcacagatatagatgaggcgttagaaataatcaaatcttttaaaggttggacaacttattttaagggttttcatgaaaatagaaaaaatgtttatagtagcaatgatattcctacatctattatttataggatagtagatgataatttgcctaaatttctagaaaataaagctaagtatgagagtttaaaagacaaagctccagaagctataaactatgaacaaattaaaaaagatttggcagaagagctaacctttgatattgactacaaaacatctgaagttaatcaaagagttttttcacttgatgaagtttttgagatagcaaactttaataattatctaaatcaaagtggtattactaaatttaatactattattggtggtaaatttgtaaatggtgaaaatacaaagagaaaaggtataaatgaatatataaatctatactcacagcaaataaatgataaaacactcaaaaaatataaaatgagtgttttatttaagcaaattttaagtgatacagaatctaaatcttttgtaattgataagttagaagatgatagtgatgtagttacaacgatgcaaagtttttatgagcaaatagcagcttttaaaacagtagaagaaaaatctattaaagaaacactatctttattatttgatgatttaaaagctcaaaaacttgatttgagtaaaatttattttaaaaatgataaatctcttactgatctatcacaacaagtttttgatgattatagtgttattggtacagcggtactagaatatataactcaacaaatagcacctaaaaatcttgataaccctagtaagaaagagcaagaattaatagccaaaaaaactgaaaaagcaaaatacttatctctagaaactataaagcttgccttagaagaatttaataagcatagagatatagataaacagtgtaggtttgaagaaatacttgcaaactttgcggctattccgatgatatttgatgaaatagctcaaaacaaagacaatttggcacagatatctatcaaatatcaaaatcaaggtaaaaaagacctacttcaagctagtgcggaagatgatgttaaagctatcaaggatcttttagatcaaactaataatctcttacataaactaaaaatatttcatattagtcagtcagaagataaggcaaatattttagacaaggatgagcatttttatctagtatttgaggagtgctactttgagctagcgaatatagtgcctctttataacaaaattagaaactatataactcaaaagccatatagtgatgagaaatttaagctcaattttgagaactcgactttggctaatggttgggataaaaataaagagcctgacaatacggcaattttatttatcaaagatgataaatattatctgggtgtgatgaataagaaaaataacaaaatatttgatgataaagctatcaaagaaaataaaggcgagggttataaaaaaattgtttataaacttttacctggcgcaaataaaatgttacctaaggttttcttttctgctaaatctataaaattttataatcctagtgaagatatacttagaataagaaatcattccacacatacaaaaaatggtagtcctcaaaaaggatatgaaaaatttgagtttaatattgaagattgccgaaaatttatagatttttataaacagtctataagtaagcatccggagtggaaagattttggatttagattttctgatactcaaagatataattctatagatgaattttatagagaagttgaaaatcaaggctacaaactaacttttgaaaatatatcagagagctatattgatagcgtagttaatcagggtaaattgtacctattccaaatctataataaagatttttcagcttatagcaaagggcgaccaaatctacatactttatattggaaagcgctgtttgatgagagaaatcttcaagatgtggtttataagctaaatggtgaggcagagcttttttatcgtaaacaatcaatacctaaaaaaatcactcacccagctaaagaggcaatagctaataaaaacaaagataatcctaaaaaagagagtgtttttgaatatgatttaatcaaagataaacgctttactgaagataagtttttctttcactgtcctattacaatcaattttaaatctagtggagctaataagtttaatgatgaaatcaatttattgctaaaagaaaaagcaaatgatgttcatatattaagtatagatagaggtgaaagacatttagcttactatactttggtagatggtaaaggcaatatcatcaaacaagatactttcaacatcattggtaatgatagaatgaaaacaaactaccatgataagcttgctgcaatagagaaagatagggattcagctaggaaagactggaaaaagataaataacatcaaagagatgaaagagggctatctatctcaggtagttcatgaaatagctaagctagttatagagtataatgctattgtggtttttgaggatttaaattttggatttaaaagagggcgtttcaaggtagagaagcaggtctatcaaaagttagaaaaaatgctaattgagaaactaaactatctagttttcaaagataatgagtttgataaaactgggggagtgcttagagcttatcagctaacagcaccttttgagacttttaaaaagatgggtaaacaaacaggtattatctactatgtaccagctggttttacttcaaaaatttgtcctgtaactggttttgtaaatcagttatatcctaagtatgaaagtgtcagcaaatctcaagagttctttagtaagtttgacaagatttgttataaccttgataagggctattttgagtttagttttgattataaaaactttggtgacaaggctgccaaaggcaagtggactatagctagctttgggagtagattgattaactttagaaattcagataaaaatcataattgggatactcgagaagtttatccaactaaagagttggagaaattgctaaaagattattctatcgaatatgggcatggcgaatgtatcaaagcagctatttgcggtgagagcgacaaaaagttttttgctaagctaactagtgtcctaaatactatcttacaaatgcgtaactcaaaaacaggtactgagttagattatctaatttcaccagtagcagatgtaaatggcaatttctttgattcgcgacaggcgccaaaaaatatgcctcaagatgctgatgccaatggtgcttatcatattgggctaaaaggtctgatgctactaggtaggatcaaaaataatcaagagggcaaaaaactcaatttggttatcaaaaatgaagagtattttgagttcgtgcagaataggaataactaaaggtcgaaattcaaattgtgagcggataacaatttgaattttctgtatgaggttttgctaaacaactttcaacagtttcagcggagtgagaatagaaaggaacaactaaaggaattgcgaataataattttttcacgttgaaaatctccaaaaaaaaaggctccaaaaggagcctttaattgtatcggtttatcagcttgctttcgaggtgaatttcgaccctctagcgaaaatgcaagagcaaagacgaaaacatgccacacatgaggaataccgattctctcattaacatattcaggccagttatctgggcttaaaagcagaagtccaacccagataacgatcatatacatggttctctccagaggttcattactgaacactcgtccgagaataacgagtggatcccctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggtgaatccgttagcgaggtgccgccggcttccattcaggtcgaggtggcccggctccatgcaccgcgacgcaacgcggggaggcagacaaggtatagggcggcgaggcggctacagccgatagtctggaacagcgcacttacgggttgctgcgcaacccaagtg

SEQ ID NO: 12 - DNA, 165 bp, synthetic construct
  misc_feature 1..165: note = Nucleotide sequence of the PsacB RNA polymerase II promotor from Bacillus megaterium
  source 1..165: mol_type = other DNA
gcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcg

SEQ ID NO: 13 - DNA, 169 bp, synthetic construct
  misc_feature 1..169: note = Nucleotide sequence of the transcription T1 and T2 termination region of the E. coli rrnB gene
  source 1..169: mol_type = other DNA
tttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttat

SEQ ID NO: 14 - DNA, 19 bp, synthetic construct
  misc_feature 1..19: note = BEC family Stem-Loop sequence
  source 1..19: mol_type = other DNA
aatttctactgttgtagat

SEQ ID NO: 15 - DNA, 24 bp, synthetic construct
  misc_feature 1..24: note = Kanamycin target-specific 24 bps spacer sequence
  source 1..24: mol_type = other DNA
tcggcaggagcaaggtgagatgac

SEQ ID NO: 16 - DNA, 5039 bp, synthetic construct
  misc_feature 1..5039: note = Nucleotide sequence of the CRISPR/gRNA-Ec vector system
  source 1..5039: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcggaattaaccctcactaaagggaacaaaagctggagctctgcagcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaatttctactgttgtagattcggcaggagcaaggtgagatgacggccggcatggtcccagcctcctcgctggcgccggctgggcaacatgcttcggcatggcgaatgggactttttttttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaaaggcaggccgggccgtggtggccacggcctctaggccagatccagcggcatctgggttagtcgagcgcgggccgcttcccatgtctcaccagggcgagcctgtttcgcgatctcagcatctgaaatcttcccggccttgcgcttcgctggggccttacccaccgccttggcgggcttcttcggtccaaaactgaacaacagatgtgtgaccttgcgcccggtctttcgctgcgcccactccacctgtagcgggctgtgctcgttgatctgcgtcacggctggatcaagcactcgcaacttgaagtccttgatcgagggataccggccttccagttgaaaccactttcgcagctggtcaatttctatttcgcgctggccgatgctgtcccattgcatgagcagctcgtaaagcctgatcgcgtgggtgctgtccatcttggccacgtcagccaaggcgtatttggtgaactgtttggtgagttccgtcaggtacggcagcatgtctttggtgaacctgagttctacacggccctcaccctcccggtagatgattgtttgcacccagccggtaatcatcacactcggtcttttccccttgccattgggctcttgggttaaccggacttcccgccgtttcaggcgcagggccgcttctttgagctggttgtaggaagattcgatagggacacccgccatcgtcgctatgtcctccgccgtcactgaatacatcacttcatcggtgacaggctcgctcctcttcacctggctaatacaggccagaacgatccgctgttcctgaacactgaggcgatacgcggcctcgaccagggcattgcttttgtaaaccattgggggtgaggccacgttcgacattccttgtgtataaggggacactgtatctgcgtcccacaatacaacaaatccgtccctttacaacaacaaatccgtcccttcttaacaacaaatccgtcccttaatggcaacaaatccgtccctttttaaactctacaggccacggattacgtggcctgtagacgtcctaaaaggtttaaaagggaaaaggaagaaaagggtggaaacgcaaaaaacgcaccactacgtggccccgttggggccgcatttgtgcccctgaaggggcgggggaggcgtctgggcaatccccgttttaccagtcccctatcgccgcctgagagggcgcaggaagcgagtaatcagggtatcgaggcggattcacccttggcgtccaaccagcggcaccagcggcgcctgagaggcctacagagcggttggacaccaaggggaggggctaagaccggtttatcagtcccctttccctcgtttctttccaacgcgatagcccagcaaggccgccaccgttgccaccgtcaccccagcaagcacagccagtggcgtgtaattggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgccgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 17 - DNA, 27 bp, synthetic construct
  misc_feature 1..27: note = SV40 nuclear localization signal (NLS)
  source 1..27: mol_type = other DNA
atggctcccaaaaagaaaaggaaggtg

SEQ ID NO: 18 - DNA, 548 bp, synthetic construct
  misc_feature 1..548: note = Nucleotide sequence of the p. pastoris HTX-Promotor
  source 1..548: mol_type = other DNA
tgatttgtttaggtaacttgaactggatgtattagtttggtgacaagcggatgtgggatatcagtgtgtttatatatacgtgatggggggaggactctcgtttcctatgataccttatggaatgggacagtacgcgctaatttgtctcgttctggatagactgctctgtttttcgttacatacgagtgggaagtgcgaacttttgtaccagagtgcgaaaaaagtggtggcaatcctgacgtttgtaccaagcgcgacggcacattattggtcagtttgtgcattttagtttttcttgaaaacatctcgaaacataacaactaggacaccaattctacagtgaaaaccattcagtggaatgtgaacccacataaatcttgcagcaataatacaacaatttatggctcctgcattctgattggctgagcgtttattgccatcgtgactgtccaatttaggacacaccctttcctcagaatatataaactggtgataattccttcgttctgagttccatctcatactcaaactatattaaaactacaaca

SEQ ID NO: 19 - DNA, 260 bp, synthetic construct
  misc_feature 1..260: note = Nucleotide sequence of the P. pastoris AOX1TT-Terminator
  source 1..260: mol_type = other DNA
tcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagcagatgaatatcttgtggtaggggtttgggaaaatcattcgagtttgatgtttttcttggtatttcccactcctcttcagagtacagaagattaagtgagaccttcgtttgtgc

SEQ ID NO: 20 - DNA, 810 bp, synthetic construct
  misc_feature 1..810: note = Nucleotide sequence of the kanMX6 marker gene
  source 1..810: mol_type = other DNA
atgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaa

SEQ ID NO: 21 - DNA, 624 bp, synthetic construct
  misc_feature 1..624: note = Nucleotide sequence of the bifunctional ILV5/synthetic Em72 Promotor
  source 1..624: mol_type = other DNA
ggatccttcagtaatgtcttgtttcttttgttgcagtggtgagccattttgacttcgtgaaagtttctttagaatagttgtttccagaggccaaacattccacccgtagtaaagtgcaagcgtaggagcaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaattttctagagtgttgttactttatacttccggctcgtataatacgacaaggtgtaaggaggactaaacc

SEQ ID NO: 22 - DNA, 156 bp, synthetic construct
  misc_feature 1..156: note = Nucleotide sequence of the pichia autonomously replicating sequence 1 (PARS1)
  source 1..156: mol_type = other DNA
caattaatatttacttattttggtcaaccccaaataggttgatttcatacttggttcattcaaaaataagtagtcttttgagatctttcaatattataataaatatactataacagccgacttgtttcattttcgcgaatgttcccccagcttatc

SEQ ID NO: 23 - DNA, 24 bp, synthetic construct
  misc_feature 1..24: note = Ade2 target-specific 24 bps spacer sequence (BEC10)
  source 1..24: mol_type = other DNA
tcaccgaaatgttgtgcttgatca

SEQ ID NO: 24 - DNA, 1692 bp, synthetic construct
  misc_feature 1..1692: note = Nucleotide sequence of the P. pastoris ade2 gene
  source 1..1692: mol_type = other DNA
atggattctcaggtaataggtattctaggaggaggccagctaggccgaatgattgttgaggccgctagcaggctcaatatcaagaccgtgattcttgatgatggtttttcacctgctaagcacattaatgctgcgcaagaccacatcgacggatcattcaaagatgaggaggctatcgccaagttagctgccaaatgtgatgttctcactgtagagattgagcatgtcaacacagatgctctaaagagagttcaagacagaactggaatcaagatatatcctttaccagagacaatcgaactaatcaaggataagtacttgcaaaaggaacatttgatcaagcacaacatttcggtgacaaagtctcagggtatagaatctaatgaaaaggcgctgcttttgtttggagaagagaatggatttccatatctgttgaagtcccggactatggcttatgatggaagaggcaattttgtagtggagtctaaagaggacatcagtaaggcattagaattcttgaaagatcgtccattgtatgccgagaagtttgctccttttgttaaagaattagcggtaatggttgtgagatcactggaaggcgaagtattctcctacccaaccgtagaaactgtgcacaaggacaatatctgtcatattgtgtatgctccggccagagttaatgacaccatccaaaagaaagctcaaatattagctgaaaacactgtgaagactttcccaggcgctggaatcttcggagttgagatgttcctattgtctgatggagaacttcttgtaaatgagattgctccaaggccccacaattctggtcactatacaatcgatgcatgtgtaacatctcagttcgaagcacatgtaagagccataactggtctgccaatgccactagatttcaccaaactatctacttccaacaccaacgctattatgctcaatgttttgggtgctgaaaaatctcacggggaattagagttttgtagaagagccttagaaacacccggtgcttctgtatatctgtacggaaagaccacccgattggctcgtaagatgggtcatatcaacataataggatcttccatgttggaagcagaacaaaagttagagtacattctagaagaatcaacccacttaccatccagtactgtatcagctgacactaaaccgttggttggagttatcatgggttcagactctgatctacctgtgatttcgaaaggttgcgatattttaaaacagtttggtgttccattcgaagttactattgtctctgctcatagaacaccacagagaatgaccagatatgcctttgaagccgctagtagaggtatcaaggctatcattgcaggtgctggtggtgctgctcatcttccaggaatggttgctgccatgactccgttgccagtcattggtgttcctgtcaagggctctacgttggatggtgtagactcgctacactcgattgtccaaatgcctagaggtgttcctgtggctacggttgctatcaacaacgccaccaatgccgctctgttggccatcaggattttaggtacaattgaccacaaatggcaaaaggaaatgtccaagtatatgaatgcaatggagaccgaagtgttggggaaggcatccaacttggaatctgaagggtatgaatcctatttgaagaatcgtctttga

SEQ ID NO: 25 - DNA, 7742 bp, synthetic construct
  misc_feature 1..7742: note = Nucleotide sequence of the CRISPR/BEC10-Pp all-in-one-vector system
  source 1..7742: mol_type = other DNA
atgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggtacctgcagctaaggtaatcagatccaagtttccccaatcttccggggcaaaccaattcaggcaatgtggtcccacgataaggccgaatataacggccacagaagcttctccgatatagagcttctctttaataaaaagagagcacagagaaaatattgtggaaaaaacacctatacatgcgtaggcaatatgggttttggagggcgctaagtgctcccatatagccatgttgtaaaggattatgcaaaatttggcaggagaatctgtggatgcaaggtataattataccgaagggaaaaaaagagagaagagcataaaaagcttatttttagttgacgttcgcggacatagattgctggcttgataagataggacttttggatagttacagaatataagggcataaatagataataaaacatccgtatacatacaataaaactaataaatactctctaaataatcgtaaggtgtcaattgttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttgccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgaaacgtgagtcttttccttacccatggtttagtcctccttacaccttgtcgtattatacgagccggaagtataaagtaacaacactctagaaaattttttcggataattttttaaagcgagaaagtcacccagcggatatgaacgcttattgtgtgccgcacatcatattggcgtttgaagtcgcggtttcgagattatctcttaatttgttcgcattttactttcaagcgcaacacaatgaagtgctctaatattacaaagattgttggaactggctcacttggaattgcaacaggcgtccttgggtatgcatgcttacgcggaatcgacaaccttgtcacgttgatcgataacaacaaactggaccaacgaatgcgaaggttagtaggacgcgttcttagatccacacggtacattgttgccatatgcatgactactggatcttatctgctagtagaaactttcagaagatcacctgccagtgctcgacacttatacctgatttatgccagtcttggtgctcctacgcttgcactttactacgggtggaatgtttggcctctggaaacaactattctaaagaaactttcacgaagtcaaaatggctcaccactgcaacaaaagaaacaagacattactgaaggatcccgccggtggataagctgggggaacattcgcgaaaatgaaacaagtcggctgttatagtatatttattataatattgaaagatctcaaaagactacttatttttgaatgaaccaagtatgaaatcaacctatttggggttgaccaaaataagtaaatattaattgcgccggtgggatccgcacaaacgaaggtctcacttaatcttctgtactctgaagaggagtgggaaataccaagaaaaacatcaaactcgaatgattttcccaaacccctaccacaagatattcatctgctgcgagataggctgatcaggagcaagctcgtacgagaagaaacaaaatgacaaaaaaaatcctatactatataggttacaaataaaaaagtatcaaaaatgaagcctgcatctctcaggcaaatggcattctgacatcctcttgagcggccgcttacttcatctgaatcaagttttcgacggacttcaaggcaatatggtaagcaccattatcatcaccattgtggatgtagtggatgttgttttccctttctttcaactgtgagtcggacctaaaaccacattctttacagaccaggatgtcatccttatctctgtaaacgttagtcttagagcaaactggacacttcttagatgtgaactttggatcgatgaaatgaacaacaccgaatggcttagacttgtacttagtctcttccaacttgatgatatttctgtagttatcggcggatctaaaagctggaacaaatctcaaaacggtgttatcagattggatcttctgcagctttttcagcagctgcatttcaaaaaattggtaagtacccaagccagccaacatttgattctgctgttctttaaagtcaacatcctcatcttgagaggtggatggcaaatatctaccatcataaccagattttgcaccaccgtaagctctggacaaatcttctaaagaaatgtaggccttgtaggagtacttggcgaaaatgtagttaacgataccgatcatgttagcaacgataccttgtttcaaattgtcggcagcatccaattcgatgatcttgtttttctcagtctgattgtttcctctggcaataacaccttgcaagtcagagatcatttccctcatggtgttaataggcaaatcagcgtacttttgaccttcaccgaatgaagaaatcaagccctcgatcctctttttgaattcttcatcagatggttcgttgttgatcaggtccaagacatcttgttcattggtctgcatcttgtgttgcaactttctaatgtaggacagctgcttcaacttaatcttctgcttgttttcttcggtagcttcttcatctggagtatctctgttcttcttgaccagtgtcaaagattgatcaaccaacaccttattatcctcaatggtagtttcaactctcaagttagacaagtccaagatatgtctggtttcagccaaggtgtgcttccagtaattcttagatctcttttcggctctgacgaactcttttttgtagatttcaaagtcacccaaaatcttacccctcttgtccaaaacacacaaagtagctaattgcttcaagcctctgtcgataccaataacaacgtattctttacccaacaaggcatggttaacctttttgttgaactcttcgacggcttctttttgagcatcttcgtcgttgaagttctcgatctgcttcttcaagttgatgaactcaccattttgtgggatgatttcgcagttaaaagcgcaaacgaattgcaatctaccgtatcttttatcctttgggtagtccttaattggagttctgtaagaaacggcgaattctggatgcaatctgaattctttggaaccttcgaagatttgcttccaatccttggagaactgattcttgtccttacgaccttgagaggtgatgtcctggttgataattggcaacaacaaacaaccaccttcaaccaaagaagcaatagattgcttggaaatggtatgcttttgcaagatgtaagacttctgatcgatttcgtgttcgatatcttcgtaggagttacacttctcgaagttccaaccaaattcagcccaattttgattcttagccatggtagaatcggtcaagcaatccttaacgtattgcaacaagacttgttctctcttgtagttgtcccattctttcttgatcttggtccaatcaacatggatcttagaagtatggaaatcagcatatggagaaattgcacccttcttggtggtatgattcttaatcagcttgatcagggtcttagaggtcaaagacttaacttggtaggtgatgaattcggatgaggtgttcaacttacccaaaatggcttcgacatggttgatttgttcgtgttgcaaaccgttggccaacaagtatctgtctttgttgttatcttcgatgataataccgaaatgggtgatcttggtgatgttgttcttttctttgaacttgtctctcaattcagcgaaggttttaccgtaggtagaagagatttgcttgaattcgttggtcaacttagtgtatgggttttcgtagtaggacttgatcttaccgtacttgtcttttctgaacatcttttgagccttgattctacctttcagaccacctctaataaaacccaactgttgtttagcatcggtgtacttttcgtgacctctaccatcgttaatccaggccaagatatccttcttgtaggtttcgaactctttaatcttgtatggcagaacgatgttctcgaaggtgttgttaatcttgcagatgaattcctcttgattagatggcaggttgttcttgtgctccaaaacagccaaattctttctgatgtacaacaggtcggcgtagaacttaaaaactggttctttggagacttcgcaatcaaaaacatccttcaagtacttctcgcactgttcttttgggaaagtaacagtagtgtagacaccgttagccaattgttcccatgcgtaatcaaaagcaaccttgattgggtagtgattcaagtcaaaaacatcttcattatgagcgtaatcatgagctggagatctagtagcaccaattgcatccaaaaccttcaaaacggcatcttcatcttcagcgatatacttagccaattcgtgcttgaccaaaaatggaatgggcttgtacttaaagcattgggttctaatgataacggagatgtttctatcatcgtagatcttgaacttgtcgaacttggaaaagtactcttcgatttgcttggacgtcttgttcttcaacttttcgaccaactcaatcaacttcaactcacggatcttggcatcaatatcgttcttgaaaacgtgtggcttttgttcagcagtgtaatggttcaaggtaaccttagcaaatggagtataaccaccattggtagcaatagcatccttcaattcaccaatagaaccaaccaaatcggagtaatcgttcagagcgaattcaatcaaatgtctggattcttcaccatcttccaacttggagatgtttggaaaagaaatagcaccgttggacaatgggatgacaatttcgttaaccaagttggccaaaatggagaaggtcttgttgaagttaatcaggtttggcttggtatgagccttatcagaattttccaaagctctcttgtactgcttcaattgttgagagaaatccttcaggaaagacttttgtctggcaatgttgtccttccagtagttggtaatatgttgagatctcttcaaaccctggtaaacgttgtgcttagaagataaggatgacaaggtgatcagttgagagttttgatcaccaacatcgaatctggcctttctagccaattgtctataccagtccttgtagacggcaatttggttagttctacagtagatttgaccccaagcttcatggaacttgataatttcggagtagcacctttcgcactttttgaccaattcgatttcgttcaaggattcagcaatggtagcagatgacttcaaggtcttgtaggaaatgtcaacgaattcttgcaattccaagtgggacttgcactttttcttatcctctttcaaagtggcaccgaatctcaaagtcttagaaatttggcaggagttttcgaaggacttcatcaccttccttttctttttgggagccatgtttcggaattctgttgtagttttaatatagtttgagtatgagatggaactcagaacgaaggaattatcaccagtttatatattctgaggaaagggtgtgtcctaaattggacagtcacgatggcaataaacgctcagccaatcagaatgcaggagccataaattgttgtattattgctgcaagatttatgtgggttcacattccactgaatggttttcactgtagaattggtgtcctagttgttatgtttcgagatgttttcaagaaaaactaaaatgcacaaactgaccaataatgtgccgtcgcgcttggtacaaacgtcaggattgccaccacttttttcgcactctggtacaaaagttcgcacttcccactcgtatgtaacgaaaaacagagcagtctatccagaacgagacaaattagcgcgtactgtcccattccataaggtatcataggaaacgagagtcctccccccatcacgtatatataaacacactgatatcccacatccgcttgtcaccaaactaatacatccagttcaagttacctaaacaaatcagatcgaaattctgatgagtccgtgaggacgaaacgagtaagctcgtcaatttctactgttgtagattcaccgaaatgttgtgcttgatcaggccggcatggtcccagcctcctcgctggcgccggctgggcaacatgcttcggcatggcgaatgggactcaagaggatgtcagaatgccatttgcctgagagatgcaggcttcatttttgatacttttttatttgtaacctatatagtataggattttttttgaaatgaataataactgtgtatttttcagtgttcccgatctgcgtctatttcacaataccaacatgagtcaaccaaatccaaatgtcatcattgtaggcgatggagatttcaggcagtcaaagcgggaattggatctgggtaccgcaggaaagaac

SEQ ID NO: 26 - DNA, 4107 bp, synthetic construct
  misc_feature 1..4107: note = Nucleotide sequence of the Codon Optimized SpCas9 Sequence for yeast
  source 1..4107: mol_type = other DNA
atggacaagaagtactccatcggtttggatattggtacaaattctgttggttgggccgttattactgatgagtacaaagttccatccaagaagttcaaggttttgggtaacactgataggcactccatcaaaaagaatttgattggtgccttgttgttcgactctggtgaaactgctgaagctactagattgaaaagaaccgctagaagaagatacaccagaagaaagaacagaatctgctacttgcaagagatcttctctaacgaaatggccaaggttgatgattcattcttccacagattggaagagtccttcttggttgaagaggataagaaacacgaaagacatccaatcttcggtaacatcgttgatgaagttgcttaccacgaaaagtacccaactatctaccatttgagaaagaagttggttgactctaccgataaggctgatttgagattgatctatttggctttggcccacatgattaagttcagaggtcatttcttgatcgagggtgatttgaatccagataactccgatgttgacaagttgttcatccaattggtccaaacttacaaccagttgttcgaagaaaatccaatcaatgcctctggtgttgatgctaaggctattttgtctgctagactgtctaagtctagaaggttggaaaacttgattgctcaattgccaggtgaaaagaagaatggtttgttcggtaatttgatcgccttgtctttgggtttgactccaaatttcaagtccaacttcgatttggctgaagatgctaagttgcaattgtctaaggatacctacgatgatgacttggataatttgttggcccaaatcggtgatcaatacgctgatttgtttttggctgctaagaacttgtccgatgccattttgttgtccgatatcttgagagttaacaccgaaattactaaggctccattgtctgcctctatgatcaaaagatacgatgaacaccaccaagacttgactttgttgaaagctttggtcagacaacagttgccagaaaagtacaaagaaatcttcttcgaccagtccaaaaatggttacgctggttatattgatggtggtgcttctcaagaagagttctacaagttcattaagcccatcttggaaaagatggatggtactgaagaactgttggtcaagttgaacagagaagatttgttgagaaagcagagaaccttcgataacggttctattccacatcaaatccacttgggtgaattgcatgctattttgagaaggcaagaggatttctacccattcttgaaggataacagagaaaagatcgagaagatcctgaccttcagaattccatattatgttggtccattggccagaggtaattctagatttgcttggatgactaggaagtccgaagaaactattactccatggaacttcgaagaggttgttgataagggtgcttctgctcaatccttcattgaaagaatgactaacttcgacaagaacctgccaaacgaaaaggttttgccaaaacactctttgctgtacgaatacttcaccgtctacaacgaattgactaaggttaagtacgtcaccgaaggtatgagaaaaccagcttttttgtctggtgaacaaaagaaggctatcgtcgatttgttgttcaagactaacagaaaggttaccgtcaagcaattgaaagaggactacttcaagaaaatcgagtgcttcgattccgttgaaatttccggtgttgaagatagattcaacgcttccttaggtacttaccatgacctgttgaaaatcatcaaggacaaggatttcttggacaacgaagaaaacgaggatatcttggaggatatcgttttgactttgaccttgttcgaggatagggaaatgatcgaagaaaggttgaaaacctacgctcacttgtttgatgataaggtcatgaagcagttgaagagaagaaggtatactggttggggtagattgtctaggaaattgattaacggtatcagggacaagcaatccggtaagactattttggactttttgaagtctgatggcttcgccaatagaaacttcatgcaattgatccacgatgacagcttgaccttcaaagaagatattcaaaaggcccaagtttccggtcaaggtgattctttacatgaacatattgctaacttggctggttccccagctattaagaaaggtattttacaaaccgttaaggtggtcgacgaattggtaaaagttatgggtagacataagccagagaacatcgttattgaaatggctagagaaaatcagactacccaaaagggtcaaaagaactccagagaaaggatgaagagaattgaagagggtatcaaagaattgggctcccagattttgaaagaacacccagttgaaaacacccaattgcagaacgaaaagctgtacttgtactacttgcagaatggtagagatatgtacgtcgaccaagaattggatatcaacagattgtccgattacgatgtcgatcatatcgttccacagtcttttttgaaggatgactccatcgataacaaggtcttgactagatccgataagaacagaggtaagtctgataacgttccatctgaagaggtcgtcaagaagatgaagaattattggagacagttgctgaacgccaagttgattactcaaagaaagttcgacaatttgaccaaggctgaaagaggtggtttgtctgaattggacaaagcaggtttcattaagaggcaattggttgaaactaggcaaatcaccaaacacgttgcccaaattttggattccagaatgaataccaagtacgacgaaaacgataagttgatcagggaagttaaggtcatcacgttgaagtctaagttggtttccgatttcagaaaggacttccaattctacaaggtcagggaaatcaacaactaccatcatgctcatgatgcttacttgaatgctgttgttggtactgccttgattaagaagtatccaaagttggaatccgagttcgtttacggtgattacaaggtttacgacgtcagaaagatgattgccaagtccgaacaagaaattggtaaggctactgccaagtactttttctactccaacatcatgaatttcttcaagaccgaaatcactttggccaacggtgaaattagaaaaaggccattgattgagactaacggtgagactggtgaaatagtttgggataagggtagagatttcgctaccgttagaaaagttttgtccatgccacaagttaacatcgtcaaaaagaccgaagttcaaactggtggtttctccaaagaatccatcttgccaaaaagaaactccgacaaattgatcgccagaaagaaagattgggacccaaaaaagtatggcggttttgattctccaactgttgcttactctgttttggttgttgccaaagtcgaaaagggtaagagtaagaagttgaagtcagtcaaagagttgttgggtatcacgatcatggaaaggtcatctttcgaaaagaatcctatcgactttttggaagccaagggttacaaagaagtcaaaaaggacctgattatcaagctgccaaagtactctttgttcgaattggaaaacggtcgtaagagaatgttggcttctgctggtgaactacaaaaaggtaatgaattggctctgccctctaagtacgttaacttcttgtacttggcttctcactacgaaaagttgaaaggttctccagaagataacgagcaaaagcagttgtttgtggaacaacataagcactacttggacgaaatcatcgaacagatctctgaattctccaagagggttattttggctgatgccaatttggataaggtcttgtctgcttacaacaagcacagagataagccaattagagaacaagccgaaaacatcattcacctgttcactttgactaatttgggtgctccagctgcttttaagtacttcgatacaaccatcgacagaaagagatacacctctaccaaagaagttttggacgctaccttgatccatcaatctattactggtctgtacgaaaccagaatcgacttgtctcaattaggtggtgattaa

SEQ ID NO: 27 - DNA, 20 bp, synthetic construct
  misc_feature 1..20: note = Ade2 target-specific 24 bps spacer sequence (Cas9)
  source 1..20: mol_type = other DNA
aagagagttcaagacagaac

SEQ ID NO: 28 - DNA, 80 bp, synthetic construct
  misc_feature 1..80: note = Nucleotide sequence of the SpCas9 specific sgRNA sequence
  source 1..80: mol_type = other DNA
gttttagagctagaaatagcaagttaaaataaggctagtccgttatcaacttgaaaaagtggcaccgagtcggtgctttt

SEQ ID NO: 29 - DNA, 8210 bp, synthetic construct
  misc_feature 1..8210: note = Nucleotide sequence of the CRISPR/Cas9-Pp all-in-one-vector system
  source 1..8210: mol_type = other DNA
atgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggtacctgcagctaaggtaatcagatccaagtttccccaatcttccggggcaaaccaattcaggcaatgtggtcccacgataaggccgaatataacggccacagaagcttctccgatatagagcttctctttaataaaaagagagcacagagaaaatattgtggaaaaaacacctatacatgcgtaggcaatatgggttttggagggcgctaagtgctcccatatagccatgttgtaaaggattatgcaaaatttggcaggagaatctgtggatgcaaggtataattataccgaagggaaaaaaagagagaagagcataaaaagcttatttttagttgacgttcgcggacatagattgctggcttgataagataggacttttggatagttacagaatataagggcataaatagataataaaacatccgtatacatacaataaaactaataaatactctctaaataatcgtaaggtgtcaattgttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttgccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgaaacgtgagtcttttccttacccatggtttagtcctccttacaccttgtcgtattatacgagccggaagtataaagtaacaacactctagaaaattttttcggataattttttaaagcgagaaagtcacccagcggatatgaacgcttattgtgtgccgcacatcatattggcgtttgaagtcgcggtttcgagattatctcttaatttgttcgcattttactttcaagcgcaacacaatgaagtgctctaatattacaaagattgttggaactggctcacttggaattgcaacaggcgtccttgggtatgcatgcttacgcggaatcgacaaccttgtcacgttgatcgataacaacaaactggaccaacgaatgcgaaggttagtaggacgcgttcttagatccacacggtacattgttgccatatgcatgactactggatcttatctgctagtagaaactttcagaagatcacctgccagtgctcgacacttatacctgatttatgccagtcttggtgctcctacgcttgcactttactacgggtggaatgtttggcctctggaaacaactattctaaagaaactttcacgaagtcaaaatggctcaccactgcaacaaaagaaacaagacattactgaaggatcccgccggtggataagctgggggaacattcgcgaaaatgaaacaagtcggctgttatagtatatttattataatattgaaagatctcaaaagactacttatttttgaatgaaccaagtatgaaatcaacctatttggggttgaccaaaataagtaaatattaattgcgccggtgggatccgcacaaacgaaggtctcacttaatcttctgtactctgaagaggagtgggaaataccaagaaaaacatcaaactcgaatgattttcccaaacccctaccacaagatattcatctgctgcgagataggctgatcaggagcaagctcgtacgagaagaaacaaaatgacaaaaaaaatcctatactatataggttacaaataaaaaagtatcaaaaatgaagcctgcatctctcaggcaaatggcattctgacatcctcttgagcggccgcctagtcgcctccaagctggctcaggtcgatcctggtctcatacaggccggtaatggactggtggatcaaggtggcatccagcacctccttagtgctagtgtaccttttcctgtcgatggtggtatcgaagtatttgaatgccgcaggtgcgcccagatttgtcagggtgaacaggtgtatgatattctcggcctgctccctgatgggtttgtctcggtgcttgttgtaggcgctgaggaccttatccaggttcgcgtcggccagaatcaccctcttgctgaactcgctgatttgctcgattatctcgtccaggtaatgcttgtgctgctccacgaacagctgcttctgctcgttgtcctcggggctgcctttcagcttttcgtagtgggacgccaggtacagaaaattcacgtacttgctgggcagcgccagttcgttgcccttttgcagttccccagcgctcgccagcatcctctttctgccgttctccaattcaaacagggagtacttggggagtttgatgatcaggtcctttttgacctctttgtagcccttggcttccaaaaagtcaatggggttcttctcaaagctggacctctccatgatggttatgccgagcagctccttcacgctcttcagtttcttgctcttccccttctctaccttcgcgaccaccagcacgctgtaggctacggtggggctgtcgaagcctccatacttcttagggtcccagtccttcttccgggcgatcagcttgtcgctgttcctcttgggcaggatggattccttgctaaagccgcctgtctgcacctcagtttttttcacaatattcacctggggcatgctcaggactttcctcacggtggcgaagtccctgcccttgtcccacacgatctcgccggtctcaccgttggtctcgataagcggcctcttcctgatttcgccgttggcaagtgtgatctcggtcttgaaaaagttcatgatgttgctgtagaagaaatacttagcggtggccttgccgatctcctgttcgctcttagcgatcatcttcctcacatcgtaaactttgtagtcgccgtagacaaattcggactccagctttggatacttcttaatcagggcggtgccgaccacggcgttcaggtatgcgtcgtgggcatggtggtagttgttgatctccctcaccttgtagaactggaaatctttgcgaaagtcggacaccaacttgctcttcagggtaatcactttcacttccctgatgagtttgtcattctcatcgtacttggtattcattctgctgtcgaggatttgggccacgtgcttcgtgatctgtcgggtctcgaccagctgcctctttatgaagcccgctttgtcaagttcggagagtccgcccctctcggccttggtcaggttatcgaactttctctgggtgatcaatttcgcgttgagcagttgcctccagtagttcttcatctttttaaccacctcttcgcttggcacgttgtcgcttttgcccctgtttttgtcggagcgggtcaggaccttgttgtcgatgctatcgtctttaagaaagctctggggcactatgtgatccacgtcatagtctgaaagcctgttgatgtccaactcctggtcaacgtacatgtccctgccgttctgcaggtagtacaggtacagcttctcgttctggagctgggtattctccacggggtgctctttgaggatctggcttccgagttccttgataccctcctcgatccttttcatgcgctcccggctgttcttctggcccttttgggtggtctggttctctctggccatctctataactatgttttcgggcttgtgcctgcccatgaccttgaccaattcgtccacaaccttaacggtctgcagtatgcccttcttgatcgcaggggagcctgccaggtttgcaatatgttcgtgcagggagtcgccctggccgctaacctgggccttctggatgtcctccttgaaggtcaagctgtcatcgtgaatcagctgcatgaagtttcggttggcgaagccgtcggacttcaggaaatccaggatggtcttgccgctctgcttgtccctaatgccgttgatgagcttcctggacagtctgccccagccggtgtatcttcgtctcttcagttgtttcatcactttgtcgtcgaacaggtgggcgtaggttttgagcctttcttcaatcatctctcggtcctcaaacagggtcaaagtcagcacgatgtcctcgagtatatcctcgttctcctcattatccaggaaatccttgtctttgattatcttcaacaagtcgtggtaggtgcccaggctggcgttgaacctgtcctccacgccgcttatttccacgctatcaaagcactcgatcttcttgaagtagtcctccttcagctgcttcacggtcaccttcctgttggtcttgaacagcaggtccacgatggccttcttctgctcgccgctcaggaaagcgggttttcgcatgccctcggtcacatatttcaccttggtgagctcgttgtacacggtgaagtactcgtaaagcaggctgtgcttgggcagcaccttctcgttaggcaggtttttgtcgaaattggtcatccgttcgatgaaagactgcgcgcttgctcctttgtccaccacttcctcgaagttccagggggtgatggtctcctcgctctttcttgtcatccaagcgaatcggctgttgcccctggcaagagggcccacgtagtaggggatcctgaaggtcagaatcttttcgattttctccctattgtccttgaggaaggggtagaaatcctcctgtcgcctcagtattgcgtgcagctcgcccaggtggatctggtgggggatgctaccgttgtcaaaggtcctctgcttccggagcaaatcttccctgttcagcttcaccagcagctcctcggtgccatccatcttctccaggatgggcttgatgaacttgtagaactcctcttggctggctccgccgtcgatgtagccggcgtagccgttcttgctctggtcgaaaaagatctccttgtacttctcgggcagttgctgcctcaccagggccttcagaagggtcaggtcctgatggtgctcgtcgtacctcttgatcatgctggcgctcaggggggccttggtgatctctgtgttcaccctaaggatatcgctaagcaggattgcgtcagaaaggttcttggcggcgaggaacaggtcagcgtactggtcgccgatttgggcgagcagattgtccaagtcgtcgtcataggtgtccttactcaactgcagcttggcatcttctgccaggtcgaagttgctcttgaagttgggggtcaggcccaggctcagggcaatcagattaccgaagaggccgttcttcttttcaccgggcagctgggctatcaggttctccaagcgcctgcttttgctcagcctggcgctaagtatggccttcgcatccaccccgctggcgttaatagggttttcctcgaacagctggttataggtctgtacaagctggatgaacaatttgtctacgtcactgttgtcggggttaaggtcgccctcgatcaggaagtggcccctgaacttaatcatgtgcgccagagccaagtagataagtcgcaagtcggctttatcggtgctatccaccaatttctttcgcaggtggtagatggtggggtacttctcgtgataagcgacctcgtcgactatgttgccgaagatggggtgtcgttcgtgcttcttatcctcctcgacaaggaagctctcctccagcctgtggaagaagctgtcgtccaccttggccatctcgttgctgaagatctcctgcagatagcagatcctgttcttcctccgggtgtaccttcgccttgcggtcctcttcaacctggtggcctcggcggtctcgcctgagtcaaacagcagagccccgataaggtttttcttgatagagtgcctgtctgtattccccagcaccttgaacttcttgctgggtaccttgtattcgtctgtgataacggcccagcccacgctattggtgccgatatcaaggccgatgctgtactttttgtccatcaccttccttttctttttgggagccatgtttcggaattctgttgtagttttaatatagtttgagtatgagatggaactcagaacgaaggaattatcaccagtttatatattctgaggaaagggtgtgtcctaaattggacagtcacgatggcaataaacgctcagccaatcagaatgcaggagccataaattgttgtattattgctgcaagatttatgtgggttcacattccactgaatggttttcactgtagaattggtgtcctagttgttatgtttcgagatgttttcaagaaaaactaaaatgcacaaactgaccaataatgtgccgtcgcgcttggtacaaacgtcaggattgccaccacttttttcgcactctggtacaaaagttcgcacttcccactcgtatgtaacgaaaaacagagcagtctatccagaacgagacaaattagcgcgtactgtcccattccataaggtatcataggaaacgagagtcctccccccatcacgtatatataaacacactgatatcccacatccgcttgtcaccaaactaatacatccagttcaagttacctaaacaaatcagatctctcttctgatgagtccgtgaggacgaaacgagtaagctcgtcaagagagttcaagacagaacgttttagagctagaaatagcaagttaaaataaggctagtccgttatcaacttgaaaaagtggcaccgagtcggtgcttttggccggcatggtcccagcctcctcgctggcgccggctgggcaacatgcttcggcatggcgaatgggactcaagaggatgtcagaatgccatttgcctgagagatgcaggcttcatttttgatacttttttatttgtaacctatatagtataggattttttttgaaatgaataataactgtgtatttttcagtgttcccgatctgcgtctatttcacaataccaacatgagtcaaccaaatccaaatgtcatcattgtaggcgatggagatttcaggcagtcaaagcgggaattggatctgggtaccgcaggaaagaac

SEQ ID NO: 30 - DNA, 1998 bp, synthetic construct
  misc_feature 1..1998: note = Nucleotide sequence of the Ade2 HDR-template for BEC10 and SpCas9
  source 1..1998: mol_type = other DNA
ctggaacaagggaattcacggtaataccttttggagcaagttcaaccgatagagccttcattaatgagttgattgcacctttggtggtcgcatataccgattgattcgggtaggtcacttcgaaactgtacagggaggcagtaaagatgatcctacccttaatctggttcttaataaagtgtttagtgactagctgtgtcaatctaaatggaaaatcgacatttaccttttggatagccgcgtaatctttctccgtaaaacttgtaaactcagatttaatggcaatggcagcgttgttgattaaaatgtcaatctttccagtggaactcttctccaccgcaggactcgttacggtctcttccagctttgcaagatcggcatccactagatccaactcaattgtatgtatggaggcaccatcggcatttgacattctcacctcttcaatgaaagccgttgggtctgtagaaggtctatggataagaataagttctgcacctgcttcataaagtcctcgaactattccttggcctaatccgctggtaccaccggtgatcaaggcgaccttaccattcaaagaaaacaaatcagcggacattagcgacttgaatagggaatgggttagacaaatgaaagccgacgagccagcactttatagtaagtgcaggtgagtcaataagaataaatgtatggcttgctgtccctatcgcgtaagaagcttactaagatcgcctaaattgaaaagttgaacaaatcagttctagctggcctccatcagcatttcgttctcctctgatcatctttgccaatcgctagcatgccctcagcgtgcaaggaaaagcacgcttctttcttatcgacgtattttcaactatggcagagccaggttagcaagtcaaagccgtatactcggtagtgtgctcgccaaaaataaatttgacttgactcttcactagcctatgcaaataaggttaccttttccaagaatcgtagaaacgattaaaaaacttccaaactctcatttagtattgttttttaatagatgtatatataatagtacacgtaacttatctattccattcataattttattttaaaggttcggtagaaatttgtcctccaaaaagttggttagagcctggcagttttgataggcattattatagattgggtaatatttaccctgcacctggaggaactttgcaaagagcctcatgtgctctaaaaggatgtcagaattccaacatttcaaaattatatctgcatgcgtctgtaatactggaactgttatttttctggtcaggatttcaccgctcttgtcgtcatgtttctcgtcgtctgaaagtaaactgactttcctctttccataaacacaaaaatcgattgcaacttggttattcttgagattgaaatttgctgtgtcttcagtgcttagctgaatatcaacaaacttacttagtactaataacgaagcactatggtaagtggcataacatagtggtattgaagcgaacagtggatattgaacccaagcattggcaacatctggctctgttgatactgatccggatcgtttggcaccaattcctgaaacggcgtagtgccaccaaggtttcgatttgagaacaggttcatcatcagagtcaaccaccccaatgtcaatggcaggctccaacgaagtaggtccaacaacaacaggaagtatttgaccttgaagatctgttcctttatgatccaccacaccttgccccaattccaataactttaccagtcccgatgcagacatgataactggtactaatgatctccattgattttcgtcggcactacgtaaagcctccaaaaatgaattcagaatatcttctgaaactagattctgcttctgtgattcaagcattgctttatgtagacatctcttgaataaaagcaattctccacatattggtgtgtgtaagatagatctggaaagatgtatctggaatagtccagtcaacgttg

SEQ ID NO: 31 - DNA, 10243 bp, synthetic construct
  source 1..10243: mol_type = other DNA; note = Nucleotide sequence of the CRISPR/BEC10-FLAG-Ec vector system
ccatgcttatctccatgcggtagggtgccgcacggttgcggcaccatgcgcaatcagctgcaacttttcggcagcgcgacaacaattatgcgttgcgtaaaagtggcagtcaattacagattttctttaacctacgcaatgagctattgcggggggtgccgcaatgagctgttgcgtaccccccttttttaagttgttgatttttaagtctttcgcatttcgccctatatctagttctttggtgcccaaagaagggcacccctgcggggttcccccacgccttcggcgcggctccccctccggcaaaaagtggcccctccggggcttgttgatcgactgcgcggccttcggccttgcccaaggtggcgctgcccccttggaacccccgcactcgccgccgtgaggctcggggggcaggcgggcgggcttcgccttcgactgcccccactcgcataggcttgggtcgttccaggcgcgtcaaggccaagccgctgcgcggtcgctgcgcgagccttgacccgccttccacttggtgtccaaccggcaagcgaagcgcgcaggccgcaggccggaggcttttccccagagaaaattaaaaaaattgatggggcaaggccgcaggccgcgcagttggagccggtgggtatgtggtcgaaggctgggtagccggtgggcaatccctgtggtcaagctcgtgggcaggcgcagcctgtccatcagcttgtccagcagggttgtccacgggccgagcgaagcgagccagccggtggccgctcgcggccatcgtccacatatccacgggctggcaagggagcgcagcgaccgcgcagggcgaagcccggagagcaagcccgtagggcgccgcagccgccgtaggcggtcacgactttgcgaagcaaagtctagtgagtatactcaagcattgagtggcccgccggaggcaccgccttgcgctgcccccgtcgagccggttggacaccaaaagggaggggcaggcatggcggcatacgcgatcatgcgatgcaagaagctggcgaaaatgggcaacgtggcggccagtctcaagcacgcctaccgcgagcgcgagacgcccaacgctgacgccagcaggacgccagagaacgagcactgggcggccagcagcaccgatgaagcgatgggccgactgcgcgagttgctgccagagaagcggcgcaaggacgctgtgttggcggtcgagtacgtcatgacggccagcccggaatggtggaagtcggccagccaagaacagcaggcggcgttcttcgagaaggcgcacaagtggctggcggacaagtacggggcggatcgcatcgtgacggccagcatccaccgtgacgaaaccagcccgcacatgaccgcgttcgtggtgccgctgacgcaggacggcaggctgtcggccaaggagttcatcggcaacaaagcgcagatgacccgcgaccagaccacgtttgcggccgctgtggccgatctagggctgcaacggggcatcgagggcagcaaggcacgtcacacgcgcattcaggcgttctacgaggccctggagcggccaccagtgggccacgtcaccatcagcccgcaagcggtcgagccacgcgcctatgcaccgcagggattggccgaaaagctgggaatctcaaagcgcgttgagacgccggaagccgtggccgaccggctgacaaaagcggttcggcaggggtatgagcctgccctacaggccgccgcaggagcgcgtgagatgcgcaagaaggccgatcaagcccaagagacggcccgagaccttcgggagcgcctgaagcccgttctggacgccctggggccgttgaatcgggatatgcaggccaaggccgccgcgatcatcaaggccgtgggcgaaaagctgctgacggaacagcgggaagtccagcgccagaaacaggcccagcgccagcaggaacgcgggcgcgcacatttccccgaaaagtgccacctgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctagctgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttggtaccgggccccccctcgagtttattttaggaggcaaaaatggctgattacaaggatgacgacgataagggagctgctgaaaacttatatttccaatccccggcagctaagaaaaagaaactggatggcagcgtcgacatgaaaagctttgaaaacagctgccagattagcaaaaccctgcgttttggtgcaaccctgaaagaggataaaaagaaatgtaaaagccacctggaactgcaagaatttgtggatattagctacaagaccctgaaaagcagcgcaaccattgcagaaagcctgaatgaaattgagctggtgaaaaaatgcgaacgctgctatagcgaaatcatcaaatttcatgaagcctggggtcagatttattgtcgtaccaatcagattgccgtgtacaaagattggtatcgtcagctggcacgtaaagcacgttttgatgttggtgatcagaatagccagctgattaccctgagcagcctgagtagcaaacataatgtttatcagggtctgaaacgcagccagcatattaccaattattggaaagataacattgcccgtcagaaaagcttcctgaaagattttagccagcagctgaaacagtataaacgtgcactggaaaatagcgataaagcacataccaaaccgaacctgatcaacttcaataaaacctttagcattctggccaacctggtgaacgaaattgttattccgctgagcaatggtgcaattagctttccgaatatcagcaaactggaagatggtgaagaaagccgtcatctgattgaatttgccctgaatgattatagcgatctggttggtagcattggcgaactgaaagatgcaattgccaccaatggtggttataccccgtttgcaaaagttaccctgaaccattataccgcagaacagaaaccgcatgtgttcaaaaatgatatcgatgccaaaatccgtgagctgaaactgattgaactggttgagaaactgaaaaacaaaaccagcaagcagatcgaagagtacttcagcaaattcgacaagttcaaaatctatgatgatcgcaacatcagcgtgattattcgtacccagtgctttaaatacaaaccgattccgtttctggtgaaacatgaactggccaaatatatcgccgaagatgaagatgcagttctgaaagttctggatgccattggtgcgacccgtagtccggcacatgattatgcacataatgaagatgtgttcgatctgaaccactatccgattaaagtggcatttgattatgcctgggaacagctggcaaatggtgtgtataccaccgtgacctttccgaaagaacagtgtgaaaaatatctgaaggatgtgtttgattgcgaggttagcaaagaaccggtgtttaaattctatgccgatctgctgtatatccgcaaaaatctggccgttctggaacataaaaacaatctgccgagcaaccaagaagagttcatttgcaaaatcaacaacaccttcgaaaacatcgtgctgccgtataaaatcaaagagttcgaaacctacaaaaaagacatcctggcctggattaatgatggtcgtggtcatgagaaatataccgatgcaaaacagcagctgggttttattcgtggtggtctgaaaggtcgtattaaagcccagaaaatgttccgcaaagataagtacggtaagatcaaaagctattatgagaacccgtataccaaactgaccaacgaatttaagcagatcagcagcacctatggcaaaacctttgccgaactgcgtgataagtttaaagagaagaacaacatcaccaaaatcacccactttggcatcatcatcgaggataataacaaagatcgttatctgctggccaatggtctgcagcatgagcagattaatcatgttgaagcaattctgggcaaactgaataccagcagcgaatttatcacctatcaggttaaaagcctgaccagtaaaaccctgattaagctgattaaaaaccataccaccaaaaagggtgcgattagcccgtatgcagattttcataccagcaaaattcatgtggactggacgaaaatcaaaaaagagtgggataactataagcgcgaacaggttctgctgcagtatgttaaagattgtctgaccgatagcaccatggccaaaaatcagaattgggcagaatttggctggaactttgaaaaatgcaacagctatgaagatatcgagcacgagattgaccagaaaagttatatcctgcagaaacacaccatctccaaacagagcattgcaagcctggttgaaggtggttgtctgctgctgccgattattaaccaggatattacctcacagggtcgcaaagacaaaaaccagtttagtaaagattggaagcagatctttgaaggcagcaaagaatttcgtctgcatccggaatttgcagttagctatcgtaccccgatcaaagattatccgaaagataaacgttatggtcgcctgcagtttgtttgtgcctttaactgtgaaatcattccgcagaatggcgaattcatcaatctgaaaaagcagattgagaacttcaacgatgaggatgcacagaaagaagccgttgaagaattcaacaaaaaagtgaatcatgccctgctgggcaaagaatatgttgtgattggtattgatcgtggcctgaaacaactggcaaccctgtgtgttctggataaacgtggtaaaatcctgggcgattttgagatctacaagaaagaattcgttcgtgccgaaaaacgcagcaaaaactattggaaacacacactggcagaaacccgtcatattctggatctgagcaatctgcgtgttgaaaccacaatcgaagataacaaagtgctggttgatcagagtctgaccctggttaaaaagaatcgtgatacaccggatgaagaagccaccgaagaaaataaacagaagatcaaactgaagcagctgagctatattcgtaaactgcagcataaaatgcagaccaatgaacaggatgttctggacctgattaataacgaaccgagcgacgaagaatttaagaaacgtattgaaggtctgattagcagctttggtgaaggtcagaaatatgcggatctgcctattaataccatgcgcgaaatgattagtgatctgcagggtgttattgcccgtggtaataatcagacagagaaaaacaagatcatcgaactggatgcagccgataatctgaaacagggtattgttgcaaacatgattggcatcgtgaactacatctttgccaaatacagctacaaagcctatatcagcctggaagatctgagccgtgcatatggtggtgcaaaaagcggttatgatggacgctatctgccgtcaaccagccaggacgaagatgtggattttaaagaacagcagaatcagatgctggcaggtctgggtacatatcagttttttgaaatgcagctgctgaagaagctgcagaaaattcagagcgataataccgttctgcgctttgttccggcatttcgtagcgcagataactatcgcaatattatcaagctggaagaaaccaaatacaaaagcaaaccgtttggcgtggtgcactttattgatccgaaatttacgagcaaaaaatgtccggtttgcagcaaaaccaatgtgtatcgtgataaagatgacattctggtgtgtaaagaatgcggttttcgtagtgatagtcagctgaaagaacgcgaaaacaacatccactatattcataatggtgatgacaacggtgcctatcatattgcactgaaaagcgtggaaaatctgatccagatgaaaaaaaggccggcggccacgaaaaaggccggccaggcaaaaaagaaaaaggctagcggcagcggcgccggctccccaaagaagaaaaggaaggttgaagaccccaagaaaaagaggaaggtgtaatggtctagaggtcgaaattcaaattgtgagcggataacaatttgaattttctgtatgaggttttgctaaacaactttcaacagtttcagtggagtgagaatagaaaggaacaactaaaggaattgcgaataataattttttcacgttgaaaatctccaaaaaaaaaggctccaaaaggagcctttaattgtatcggtttatcagcttgctttcgaggtgaattttgaccctctagcgaaaatgcaagagcaaagacgaaaacatgccacacatgaggaataccgattctctcattaacatattcaggccagttatctgggcttaaaagcagaagtccaacccagataacgatcatatacatggttctctccagaggttcattactgaacactcgtccgagaataacgagtggatcccctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggtgaatccgttagcgaggtgccgccggcttccattcaggtcgaggtggcccggctccatgcaccgcgacgcaacgcggggaggcagacaaggtatagggcggcgaggcggctacagccgatagtctggaacagcgcacttacgggttgctgcgcaacccaagtgctaccggcgcggcagcgtgacccgtgtcggcggctccaacggctcgccatcgtccagaaaacacggctcatcgggcatcggcaggcgctgctgcccgcgccgttcccattcctccgtttcggtcaaggctggcaggtctggttccatgcccggaatgccgggctggctgggcggctcctcgccggggccggtcggtagttgctgctcgcccggatacagggtcgggatgcggcgcaggtcgccatgccccaacagcgattcgtcctggtcgtcgtgatcaaccaccacggcggcactgaacaccgacaggcgcaactggtcgcggggctggccccacgccacgcggtcattgaccacgtaggccgacacggtgccggggccgttgagcttcacgacggagatccagcgctcggccaccaagtccttgactgcgtattggaccgtccgcaaagaacgtccgatgagcttggaaagtgtcttttggctgaccaccacggcgttctggtggcccatctgcgccacgaggtgatgcagcagcattgccgccgtgggtttcctcgcaataagcccggcccacgcctcatgcgctttgcgttccgtttgcacccagtgaccgggcttgttcttggcttgaatgccgatttctctggactgcgtgg

SEQ ID NO: 32 - DNA, 21 bp, synthetic construct
  source 1..21: mol_type = other DNA; note = EGFP target-specific 21 bps spacer sequence
cgtcgccgtccagctcgacca

SEQ ID NO: 33 - DNA, 21 bp, synthetic construct
  source 1..21: mol_type = other DNA; note = Negative Control 21 bps spacer sequence
actcttgttgtgagggttgac

SEQ ID NO: 34 - DNA, 5036 bp, synthetic construct
  source 1..5036: mol_type = other DNA; note = Nucleotide sequence of the CRISPR/gRNA-EGFP-Ec vector system
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcggaattaaccctcactaaagggaacaaaagctggagctctgcagcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaatttctactgttgtagatcgtcgccgtccagctcgaccaggccggcatggtcccagcctcctcgctggcgccggctgggcaacatgcttcggcatggcgaatgggactttttttttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaaaggcaggccgggccgtggtggccacggcctctaggccagatccagcggcatctgggttagtcgagcgcgggccgcttcccatgtctcaccagggcgagcctgtttcgcgatctcagcatctgaaatcttcccggccttgcgcttcgctggggccttacccaccgccttggcgggcttcttcggtccaaaactgaacaacagatgtgtgaccttgcgcccggtctttcgctgcgcccactccacctgtagcgggctgtgctcgttgatctgcgtcacggctggatcaagcactcgcaacttgaagtccttgatcgagggataccggccttccagttgaaaccactttcgcagctggtcaatttctatttcgcgctggccgatgctgtcccattgcatgagcagctcgtaaagcctgatcgcgtgggtgctgtccatcttggccacgtcagccaaggcgtatttggtgaactgtttggtgagttccgtcaggtacggcagcatgtctttggtgaacctgagttctacacggccctcaccctcccggtagatgattgtttgcacccagccggtaatcatcacactcggtcttttccccttgccattgggctcttgggttaaccggacttcccgccgtttcaggcgcagggccgcttctttgagctggttgtaggaagattcgatagggacacccgccatcgtcgctatgtcctccgccgtcactgaatacatcacttcatcggtgacaggctcgctcctcttcacctggctaatacaggccagaacgatccgctgttcctgaacactgaggcgatacgcggcctcgaccagggcattgcttttgtaaaccattgggggtgaggccacgttcgacattccttgtgtataaggggacactgtatctgcgtcccacaatacaacaaatccgtccctttacaacaacaaatccgtcccttcttaacaacaaatccgtcccttaatggcaacaaatccgtccctttttaaactctacaggccacggattacgtggcctgtagacgtcctaaaaggtttaaaagggaaaaggaagaaaagggtggaaacgcaaaaaacgcaccactacgtggccccgttggggccgcatttgtgcccctgaaggggcgggggaggcgtctgggcaatccccgttttaccagtcccctatcgccgcctgagagggcgcaggaagcgagtaatcagggtatcgaggcggattcacccttggcgtccaaccagcggcaccagcggcgcctgagaggcctacagagcggttggacaccaaggggaggggctaagaccggtttatcagtcccctttccctcgtttctttccaacgcgatagcccagcaaggccgccaccgttgccaccgtcaccccagcaagcacagccagtggcgtgtaattggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgccgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 35 - DNA, 5036 bp, synthetic construct
  source 1..5036: mol_type = other DNA; note = Nucleotide sequence of the CRISPR/gRNA-NC-Ec vector system
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcggaattaaccctcactaaagggaacaaaagctggagctctgcagcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaatttctactgttgtagatactcttgttgtgagggttgacggccggcatggtcccagcctcctcgctggcgccggctgggcaacatgcttcggcatggcgaatgggactttttttttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaaaggcaggccgggccgtggtggccacggcctctaggccagatccagcggcatctgggttagtcgagcgcgggccgcttcccatgtctcaccagggcgagcctgtttcgcgatctcagcatctgaaatcttcccggccttgcgcttcgctggggccttacccaccgccttggcgggcttcttcggtccaaaactgaacaacagatgtgtgaccttgcgcccggtctttcgctgcgcccactccacctgtagcgggctgtgctcgttgatctgcgtcacggctggatcaagcactcgcaacttgaagtccttgatcgagggataccggccttccagttgaaaccactttcgcagctggtcaatttctatttcgcgctggccgatgctgtcccattgcatgagcagctcgtaaagcctgatcgcgtgggtgctgtccatcttggccacgtcagccaaggcgtatttggtgaactgtttggtgagttccgtcaggtacggcagcatgtctttggtgaacctgagttctacacggccctcaccctcccggtagatgattgtttgcacccagccggtaatcatcacactcggtcttttccccttgccattgggctcttgggttaaccggacttcccgccgtttcaggcgcagggccgcttctttgagctggttgtaggaagattcgatagggacacccgccatcgtcgctatgtcctccgccgtcactgaatacatcacttcatcggtgacaggctcgctcctcttcacctggctaatacaggccagaacgatccgctgttcctgaacactgaggcgatacgcggcctcgaccagggcattgcttttgtaaaccattgggggtgaggccacgttcgacattccttgtgtataaggggacactgtatctgcgtcccacaatacaacaaatccgtccctttacaacaacaaatccgtcccttcttaacaacaaatccgtcccttaatggcaacaaatccgtccctttttaaactctacaggccacggattacgtggcctgtagacgtcctaaaaggtttaaaagggaaaaggaagaaaagggtggaaacgcaaaaaacgcaccactacgtggccccgttggggccgcatttgtgcccctgaaggggcgggggaggcgtctgggcaatccccgttttaccagtcccctatcgccgcctgagagggcgcaggaagcgagtaatcagggtatcgaggcggattcacccttggcgtccaaccagcggcaccagcggcgcctgagaggcctacagagcggttggacaccaaggggaggggctaagaccggtttatcagtcccctttccctcgtttctttccaacgcgatagcccagcaaggccgccaccgttgccaccgtcaccccagcaagcacagccagtggcgtgtaattggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgccgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 36 - DNA, 720 bp, synthetic construct
  source 1..720: mol_type = other DNA; note = Nucleotide sequence of the EGFP DNA fragment
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 37 - DNA, 678 bp, synthetic construct
  source 1..678: mol_type = other DNA; note = Nucleotide sequence of the DsRed DNA fragment
atggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtag